SEQUENCE LISTING (77 sequences)

SEQ ID NO: 1 - DNA, 30 bp, synthetic construct
  source 1..30: mol_type = other DNA; note = junction sequence
ttacatgtaaggccgttcatgtccgggaaa

SEQ ID NO: 2 - DNA, 30 bp, synthetic construct
  source 1..30: mol_type = other DNA; note = junction sequence
aaactatcagtgtttcatgaagaggtatgt

SEQ ID NO: 3 - DNA, 60 bp, synthetic construct
  source 1..60: mol_type = other DNA; note = junction sequence
tctaatagaaaaggattacatgtaaggccgttcatgtccgggaaatctacatggatcagc

SEQ ID NO: 4 - DNA, 60 bp, synthetic construct
  source 1..60: mol_type = other DNA; note = junction sequence
tcccgccttcagtttaaactatcagtgtttcatgaagaggtatgtgcacgaagttcaaga

SEQ ID NO: 5 - DNA, 100 bp, synthetic construct
  source 1..100: mol_type = other DNA; note = junction sequence
atcctagtggatgaaaatgctctaatagaaaaggattacatgtaaggccgttcatgtccgggaaatctacatggatcagcaatgagtatgatggtcaata

SEQ ID NO: 6 - DNA, 100 bp, synthetic construct
  source 1..100: mol_type = other DNA; note = junction sequence
atggggatcagattgtcgtttcccgccttcagtttaaactatcagtgtttcatgaagaggtatgtgcacgaagttcaagaattggtaggaattcgttgaa

SEQ ID NO: 7 - DNA, 1050 bp, synthetic construct
  source 1..1050: mol_type = other DNA; note = 5' flank + 5' end of T-DNA insert
aaacatgataatatgaggtattatataaatagtgtgtcaatttttaggtttgcattaattgttggtcgaatttcgagaaattaaatgtgattttaggcctatttcggccttcttccatccaggggtgagggagggatgtggtggggaggggagcttggcaggggtgtgggaggtggtatgtgcaaggaggtggggaggtgggcaggggtgtggggtgggaaggggtgtgagatgtttgtgggtggccaggggcgtagagttgggaggtgtgcaaaggtggggatatgtgggcaggtgtgggttagggaggtgtggggtggggaggtgtgggtgtggagaggtgggtatgggtgtgtgtaaggtgggaacaggtagtctgtgcacctgtacgcaccctacacatacctacacccacctccccacacccacacccctacaccttcttacccctcgcgcacccacctccccaccccctgcataccccttctgcacgccttgtacacctcccccacccctcccccacccctcccccaacctcttccccaaccctacccaccctcccccaacccttccccacccctcccccacctttgtccacctcccctaacccccttcccacccatcccccacccctgcccacctcccacaaacccttatccaccctccctgcccatccctgcccacctcccccaactccctgcccacccctcccctacccctacccctacccctaccccttgtcctttagtgcggcttgtaattttttccttgagctatatatattaatgtatatttcgtataatttcagggagatggttgatttgcaaaaggcgaggagctcgggaattaaaaacactaccgaggacgagtcatatgatgagagaaaggaacaatataaaggtgatatggaggatcgagatcttgatggtgacttcgttactagttttgagatcctagtggatgaaaatgctctaatagaaaaggattacatgtaaggccgttcatgtccgggaaatctacatggatcagcaatgagtatgatggtcaata

SEQ ID NO: 8 - DNA, 1050 bp, synthetic construct
  source 1..1050: mol_type = other DNA; note = 3' end of T-DNA insert + 3' flank
atggggatcagattgtcgtttcccgccttcagtttaaactatcagtgtttcatgaagaggtatgtgcacgaagttcaagaattggtaggaattcgttgaagaaaaaaaagagaaaaggaaggggacctacaaagtcaatagggaagcataccctaatgactttggagttggtaaaaccttgtggaaagtggcggcataagtatggcatgcaaattggtattgatataaaaattggtcttggcatgcaaattggtctttgcatgcaaaaacttcccattgtacactcatgaaaagaaatacccgagggcttaaagaagtgtctatgggagcatactatggtaatgttcacgtacaataacgtttacattcttcattatttagttatatatgagtgtaatattaatgatatcatataaattacaacaattgcggaaactaatatgtgaaatgcgttttattgtagaatcttttcaatatcgaagatgatgaagcaaagaagagcctatttctatatgccgttgccgagagattcagagatttcaaggctaagttagtgtccgggtggatcactaagcggaggaatgttgtaggtacaaagaagaagaaaatatacggcatccggttatcacgcttcaccgaaagaatcaaccgagccttccaaattaccatttcatatttggaagattatcaaacctacagagtgggcggcatttgttgccaaaaacactacaccacaagctattgtaagaatattatactaacatttaactacagttaactcaaatgtttttgtattaacctttgtttgtaaattgtgttttaggcaagacgtgaaaaaagtagtgcaagcacaaaagctaggaagtattatcatcatttgggtgcgcattcgtacaatgaggcgagagaggatgttcgcccattaatccacaatggggatgacaacggcaacaaagactagctcaaccaccacagctacacagactcgacatccagataaagtcctcgatttgtggtgtttgatgcatagggttgatgcatagggaaaaa

SEQ ID NO: 9 - DNA, 11722 bp, synthetic construct
  source 1..11722: mol_type = other DNA; note = T-DNA insert
ttcatgtccgggaaatctacatggatcagcaatgagtatgatggtcaatatggagaaaaagaaagagtaattaccaattttttttcaattcaaaaatgtagatgtccgcagcgttattataaaatgaaagtacattttgataaaacgacaaattacgatccgtcgtatttataggcgaaagcaataaacaaattattctaattcggaaatctttatttcgacgtgtctacattcacgtccaaatgggggcttagatgagaaacttcacgatcgatgcggccgctctagaattcgagctctccggagtcgactttcacctttggacaatcattgacagtaacacaacgaacattaactgcatcgtaaaataaaatgtcactacaatttgataaaatattgtgtaaaagagagtacttaaaaaaaaatggcgtagaaagtaccatactcctattagctttcaacaagaaatagggaaagaaagatgactaaaacataaataaagaaagataggaaattgagccatattgttttcctttcttagatgtttcatgtggatattattttacttgatccttgtggaatttatgttatgctattattaactcttgtgtataaatagaatacaataccaatacttaaaatagacataaaaaacgatgtgagacaatagataattaaaccattcttacataatttattccttacaaaatataattcacagaatttacttttacataaaacagaacgcacacacaacacacttcacaatattctcaacctaaaaacagagtaatagagcttgaatatgtgtttaattaatcaaatctgggtaactggtctaacgggtcttggaggtgctggaagttcgaaatcccgttgccagaatccaacgtcgtgccatcctccgtgtttgtacccggcggccctcaatgttccgcgtgccgtgtagcctagtgcttcatgtaggcgcacagaaggatcgttaggtagtccgatgacggccaccactgacttaaagccctgagcctccatagatttgagtaagtgggtgtacagggtgctgcccagacccagcctctgatggcgatgcgagacgtaaacagtggattccacagtccagtcgtaagcatttcgagccttccacggtccagcgtaggcgattcccgcgacgacgccttcgacctctgccacgagccacggatagcggtcttgtagtctctccaagtcatcaatccattcctgaggagtttgtggttcggtacgaaagtttactgtggatgtctcgatgtagtgatttacgatgtcacatactgccgccatgtcggcggctgttgcgggtctaatctctactggccgtcgctcgggagacatggtaccaggttgagtagtgcagcacaaagtaaaacgtttaagatttgttgttgtaagccaagaaatgggtggtttggtattgatgcgattttcgctatatataatacagaaatctaaatactgtcttatcttgaaatatcaatctctgaggttgctattggctagtcatagattttttttggggtttagattgtttgtttgcttacgtggttaatggctcgcacttcgcagattcgcaattgatatgtgttttagatatctaggtgcgtttcatggattggcgaaacacccactcatgacgtgacacgtatacgaatagatctctctatccttatgaaatcctgagagcatcaatgtaaaccaaaacaaaaaaaatttggataattgccttgtataatacattttattaaaccttttgtatatatgcttcttttttttttcttcttctgataataataataactcttggctagagataaaacataagaacaaagtagataaaactataaaatttatgtatggaaataagattatctaaaactacggaattgaatcaaactttaccacagaaacataagaaattattaccatcttaattagcacctgtattgaccataatattttattagaagaagaagaaaacattggaatccaccagatcaaggtgatggtcagggtcataagacagctccataatcacgagtcatggtcatagactcatagccacaagtctgcttctcccttaccaataccaacggcttcaacaacggccacaactaataagccaattccaacggccaccatataactgaaccggatattcaccaaacccatttcaaccggaaattgatccacaagtaattaaaaatgttctgaccaaatgtggaataggtgggaaaatgttgaagtaaaggccaattctataatgtgagaggaaaactcttttttgagcttcaggaatattttcttagacctgaccttgaatctatattttgtagttcttcaatgatttgatttctctattcatttccttataaattaaattacttgtgaaatgaatttctgtttagaaaaaccttgaacttcctagttgaccaaaggattagaaaatctttctaagtggagtgtctcctaataatggatcaaaagtgtttggagcaaaaaagaacgcatagtgtattagtgttaacagcttttatagttcaaagtttagaatgtgctctaaagcatatgtgtgagcctgtgaggtctggtgattgtaaaaaagaaaaattgtgcttcgtggaaagtgatgcaataaacaacacttctagggctgagtaaaaccaactagacatgtaatgatagcttaaaccgttttctataggagtgtgattgaggagtaatttgttgatggatcccaagttgtttttccaagtcataaatcatttcccagtgattataaaaagcagagcagcttatccaaactcttgatttgtactataaactttcatgaaaagattgtactttcaaatggtaaagactaaaatttcaagattgtaaatgagtgaagaaacaaggttttataatgttttttaattatatatattgtgcagatgaacattgcattaacggttttgtttcatttactttatcccatttatcgtcatttataaaagatatagacacaatattattttataaaaaagtttggatttttaatcacgccacgattttttcagaacttcaatatcaattttgaatcaattttcaacattaacgattttttggataattataaaaaataacaaacatatatctcttagagatttattactgcataaacacacaaatacactttctagtaaccaaaatagtacaaattcaaagccatgaaatttatgagcttgattattcatgcactagttcacgccgcttctagttgttcgagcttctcaatctccctggataccctgacggctgcctcgtcgcaggacaacatggcgggtctgatcccgttcgcttcgacgtaagccctccgcctttcgatagcttcaacgaccactttgtcctccttcaccagggcttgagcttgccagctcctgagaacaccgtccatctctggatcgtctataccgaagttgcgggaactaccgaagaagtaatggcagcttgcctcggtctcaggagtaaggatatgggttcctcgggagtggattgactgctccttcggcgtcccttccggcgcgaccgcgatgaagttcagcatagccgacaccttattccaccggatgtcgttccaggcgtcaactggtgtgttagcaccacgcaagaacttagccatgagaactgagggtgtgccaccagggatcttcatgagcgcctggatctcgccgtcgccaacgatcacctcacgctcaagtctatcgaaggcgtctgtttgggcgttagcgcggtgcacgtactgagcgtgtccaagatccatgaggttatccacaaggagcttgtagttgcagtccacgtgaccgtaacctccgacagtacggtaagctggatcaacacgacacccaaagtcagggatagcaccgggatctgcgagtgctggatctccaggccagatccagatcaatgcgtctctttccacgacagggaatgatcgcacgttcagagaagcagggcgggctccgttgccgtgcgggttgtggacacactggccacctccgtcaaattccagaccgtgatatggacactggagatgtccgttgactagaataccgtcactcaacggagcgaagcgatgcggacaaatatcaagcagggcagcgacaactccgtcaggttgacgatagagagctagtggagtatcgaggatagttcttcctagaggcttctcgctcaactcctcaggaagtgcagcaacgtaccaagcgtttctaacgaaagtgagcatggcccgggaatctctcgtcaatggtggcaaataggaaagagtctcaaacttcttctttccaattggaggccacacctgcatgcactttactcttccaccattgcttgtaatggaagtaatgtcagtgttgaccttcctcactgggaatccagtcatggatttgaggccgccgaatggagccattgcggcggattgccccctagaggcacggctgactgttgtcacagcggaagaggatatcatagaagccatggtgcgatcgccacctgtcatgaccccaattaaacaaaaacaatttaacaattgaagatagatcgcgcaatcaagaatactctggaaaaacgttactcagatagcaaatcatatttcctacaatgcaatttaaatctgaaagacgtccgtcgaagaacataattctagtaaccagattcaatacatgtataaaaaaccaagaaccatggggaatcgataaattacaaatcttgagaagaaaaaaagataaaaatcatgataatcacaacatagaaagaagatgacaaaaaccaaatgtgaatcatccgaacaacgtcggacatcacgattaaaaatcgacctacctaacgaacagaaatgaaaaatacaacaaagacttcaaatcgtttctcgaaaattcaaacatcaattaatcgaacaggagggggaaaagatgcaatctgaaaacagaagatagagaaaagatgaaaaccttgagaggttgaagaagagcgaattacgaaataggaataaagcgcgatgagaaagtttagagtatttcgagtctgtatggaaaattagcgctcgagaattcacgtatttatacctaaaattgccgacttccgttaatcacgaataactataatattctataggaaaatgaaacgtgtctttgattacttgcgtgttacgtattgtttagttctggaagaatctagttggaaaggtcaattttccatttatacgattacgcgcagttgggaaagctttatggggaaagcataaagattaggaagttagagcagctcaattagctttattcacctctcaaatgggaaaaaggaaggaaaaaacaatacgattccttttattttctttttcttttttctaatttttctattaataaatttcatttgaatttagattttctgttcttaattttgatgtttgtatgtatttggttcaataaaattttgtatttgacaagtctaacaaatttgaaacctataaaaattaattaaatcctttatatattagtttgcattttgtgtcataaaatattagatgctatctttcacttccatagtttttcatgtaaaacctagattttttgtatgtgtatcaattaacacattcttcaaactttgaaaaatagcatacaaaatatattattttaattaaacttctaaattcattttattaagattacatatgaaaatagttcatggattcaattattacactttataggagtttaattgataacactataagaattgcttattatccaatgaatggggtaaatacaaaatttcaaattttggtaggcacgtgttaatccaaagatactaatctcggtttcaacaacagtaacctctcgatcttaactagctcaagatatgcaaccgaaaggaaatataaaaggcataaatgatacctaatctcttgacaagcagatttctagagcttagtaagaaaatatttatggagattacaaggaatagcaaaagaacataacaaatttattgtggttcgactaatagtgcctacatccactttgaagccgccatagagcttttattcaacttattcaaggatcaaacaagtatagagaatttgaaaatgagttttcttcctctctctctttaactttttctttttctttttctcattctatatttcatattgagttaaatggtttattatttacattacagagttatttaatggcagtaaatataacaaactctaactaacttattagtttttctatttgatttcagttgtaggatcacgaagctcaaaatctgtatttaggggtaccgagttgtcctgagaagtaattgcttcttcatcgagtagacagggaacaaagcatcaggtgcgtcgagattgaaccgagattgagccccacagtacaacttgcttagcttgttgaagagttagggcactgggattgtatcaagatctctcgagcttcaccaggttcttaagcaatagagtttcgaatcgagatctttcgagtttcaccgagattttctttaagtaataaaattcactcttctttttaacaatcctccaccttgaattttattgctaccaatgactatttcttctgattctattcttttaagtagtctattaccttgagcaactgtaggcagtgctcgagtttagcattagtaactattttagtcaacatatctgctgcattttcttttgtctgaacctttgatactgttacttttctctttgctatgacttctctaatgacatatagcttcacatcaatatgcttagttctttcgtgatattgcagatttttagacaagtgtattgcactttaactatcacacatgatgtttactgactgttaaaagaagccaagttcttcaagtagtccttttagccagacgacttctttaacaacttcagtcatggctataaactttacttttgttgtgaatagtgccactacaggctataggttaaacttccataaaatcacatttcctcttaggataaaagtaaaagcagcgagtgatcttcttttgtccaggtcttctgcataaccatacaactctagctttgtagacttagtatgcttgtagagtagccccttgctaggtgttcctttcagatgtggcgcgccggcaagtctttcgacccttgttcgtcactgctgacgtctttttctccattattggaggatgagtcactgcaagctgtaaagcttgtattattgctagtggaggccccacctgcacatgccgagcaattatgattgtcggccagtagctttttgagctgtttgcagagcaataacttagtggatgtccccaccattacatcgttattgatgatgctttcttcaaaggaagataagatgctgacatcctgtctggagattgtcgctccatagtgactcgtgatcaatactctttcatgtgagtaattgatttttatctttgtttctccgttgatccgcggtggatcgaccatgacattcaaaaacctttaagatatggatcttataaaataaatgtaaagggtaaacaattcttccttgcttaacccaagctatatattttatgcactaatttaggtattagagtatattcagctgaacaccacctaccaatgctagtactttaatcagtcaattctaacttcgataatatatctcaaccaaattagtgaaaaagagtcgtaaatgaaaaactatgtaccaagatattctatttgttttttttatgtttaaatatctcaaagataatacctaaaacgttttctcctcgtacaaagattcctcatttactttttattgtcgtaaactctaatacaataaactaaaacaagtacaaatacactagctttagaaatctactttttattgaaaccaaaaccaataattcaacatttcattttcaccgacaaacctttgtaaacaattgaagtaatttttgttggtactatgaatagtaacatcaagtcttcaagtgcatcatatcaaccaagacatgttcttaaaagcgacactaaaagatttaaaaccaaaagcatttatgaaatccgaacttaatcaaatcctaaatatttttcacttaaaaaaaaaaaaataggaagaaaaattgacataaatgggatattttcgttttcaaactggcaagccagcatgcaccacgttgttgacgtgtccttccacgtcggaaaaaaaaatattaccacagtaaaaagagaataaaatgaaagtcgttgactctcccttagtcggaggaagcgcgtgaagctgaagccggattagaaatcggcaataaccccgacacgtcatcgaaatgctagtatcaaatattgtccgttggatcttccttcaccaactctatttgaacggccacgatcttccaggtccaacggttcggaagaatcttttcgaaattccatggctagtccctacactcctccctattggctccctagggcatcccgaccggttattccggttgccgggaaggtggctggacgctataaatacccgctttgttcatctcgtagtccttgtaccgttgagcttcgccttctaatagagctctggttcggttggcgtattagctcgaattctttctctcttccagatctacgctgccgatttcatcaggtttgcgagctctgttccaccatttttcttttcctgaagctttgagcatgcttgtgattcttcatttcctcatttctttgatggtttatgaaagaatttaggggaattttctctttttgtattctagtggtactggtagatttgtttgaagtttgtttctcttcttctgagaagtgaattcttccagatctgacagttgcttttgattttttctttgggaattagtgaatgatacttcgatactgttttttgctctctgagattctggatctcgggccttggggttttctattgtcttttggtagctatgtttcgtttgtcagcttgtatttgtcattgttgaatggttcgatccggtttgtaaataaaataaattttgtaggcgcacttgttttccacggttttcgtgttacggtttcatgattccctagatctctggttagaactaagttttttgtcggtaattggatttggtaagggactgttactgtggttgaattgtagatccagtcatcttctacatgagtgtagggttccttagggcagatcttgtgttttataattttaattttgttgtttccctgattttgaacctgtttggttgttcagattcgtcgagtcatttccattcattaaaagtttctataattttatttgaatcttctgaatctgtgcttgtattacccagatttctataaacctatcttgatttcaagtgtgctatgtggtaactgttgatattttcaagcttaagcaatactgatgtgactaaaacttaactaatgaactgaatgttttttgtacacgaactaatatggtgttttgttatgtttcagaggacgcgtaccatggcgcaagttagcagaatctgcaatggtgtgcagaacccatctcttatctccaatctctcgaaatccagtcaacgcaaatctcccttatcggtttctctgaagacgcagcagcatccacgagcttatccgatttcgtcgtcgtggggattgaagaagagtgggatgacgttaattggctctgagcttcgtcctcttaaggtcatgtcttctgtttccacggcgtgcatgcttcacggtgcaagcagccgtccagcaactgctcgtaagtcctctggtctttctggaaccgtccgtattccaggtgacaagtctatctcccacaggtccttcatgtttggaggtctcgctagcggtgaaactcgtatcaccggtcttttggaaggtgaagatgttatcaacactggtaaggctatgcaagctatgggtgccagaatccgtaaggaaggtgatacttggatcattgatggtgttggtaacggtggactccttgctcctgaggctcctctcgatttcggtaacgctgcaactggttgccgtttgactatgggtcttgttggtgtttacgatttcgatagcactttcattggtgacgcttctctcactaagcgtccaatgggtcgtgtgttgaacccacttcgcgaaatgggtgtgcaggtgaagtctgaagacggtgatcgtcttccagttaccttgcgtggaccaaagactccaacgccaatcacctacagggtacctatggcttccgctcaagtgaagtccgctgttctgcttgctggtctcaacaccccaggtatcaccactgttatcgagccaatcatgactcgtgaccacactgaaaagatgcttcaaggttttggtgctaaccttaccgttgagactgatgctgacggtgtgcgtaccatccgtcttgaaggtcgtggtaagctcaccggtcaagtgattgatgttccaggtgatccatcctctactgctttcccattggttgctgccttgcttgttccaggttccgacgtcaccatccttaacgttttgatgaacccaacccgtactggtctcatcttgactctgcaggaaatgggtgccgacatcgaagtgatcaacccacgtcttgctggtggagaagacgtggctgacttgcgtgttcgttcttctactttgaagggtgttactgttccagaagaccgtgctccttctatgatcgacgagtatccaattctcgctgttgcagctgcattcgctgaaggtgctaccgttatgaacggtttggaagaactccgtgttaaggaaagcgaccgtctttctgctgtcgcaaacggtctcaagctcaacggtgttgattgcgatgaaggtgagacttctctcgtcgtgcgtggtcgtcctgacggtaagggtctcggtaacgcttctggagcagctgtcgctacccacctcgatcaccgtatcgctatgagcttcctcgttatgggtctcgtttctgaaaaccctgttactgttgatgatgctactatgatcgctactagcttcccagagttcatggatttgatggctggtcttggagctaagatcgaactctccgacactaaggctgcttgaaggcctttaatcatctgaaactgttcaccatgcatgcaatcttgtgaaatatatggttttaattagacttcaatcttatgttggctattgtactaataaaagcatgtcatgttattttcatttgattttatctgtactttggtttgtttgaagaataaagatgagcttgctatgcatgcatgcatgccatcgattatcagggtttccttttttcttttctggcttcccatcaatttggtgtgaattagtgtgtgtgatatattatattatgctatttatgaaataaattgttggttatatttgatctacaatctacatacatgtgatttttatcaacaaaatatctcgggaaacaatacctttttggtagcaaaattcaaataatactattttaaataaatcaaagttaaccaataccttattcaagttggaggggtctcaaacaagcaaaagaattcaagttgttaatgaacttcggttaatgataaaagaattcgcatttaaaacctgcaggtccggagagctcatttaaataagcttgatggggatcagattgtcgtttcccgccttcagtttaaactatcagtgttt

SEQ ID NO: 10 - DNA, 13722 bp, synthetic construct
  source 1..13722: mol_type = other DNA; note = event sequence (5' flank + T-DNA insert + 3' flank)
aaacatgataatatgaggtattatataaatagtgtgtcaatttttaggtttgcattaattgttggtcgaatttcgagaaattaaatgtgattttaggcctatttcggccttcttccatccaggggtgagggagggatgtggtggggaggggagcttggcaggggtgtgggaggtggtatgtgcaaggaggtggggaggtgggcaggggtgtggggtgggaaggggtgtgagatgtttgtgggtggccaggggcgtagagttgggaggtgtgcaaaggtggggatatgtgggcaggtgtgggttagggaggtgtggggtggggaggtgtgggtgtggagaggtgggtatgggtgtgtgtaaggtgggaacaggtagtctgtgcacctgtacgcaccctacacatacctacacccacctccccacacccacacccctacaccttcttacccctcgcgcacccacctccccaccccctgcataccccttctgcacgccttgtacacctcccccacccctcccccacccctcccccaacctcttccccaaccctacccaccctcccccaacccttccccacccctcccccacctttgtccacctcccctaacccccttcccacccatcccccacccctgcccacctcccacaaacccttatccaccctccctgcccatccctgcccacctcccccaactccctgcccacccctcccctacccctacccctacccctaccccttgtcctttagtgcggcttgtaattttttccttgagctatatatattaatgtatatttcgtataatttcagggagatggttgatttgcaaaaggcgaggagctcgggaattaaaaacactaccgaggacgagtcatatgatgagagaaaggaacaatataaaggtgatatggaggatcgagatcttgatggtgacttcgttactagttttgagatcctagtggatgaaaatgctctaatagaaaaggattacatgtaaggccgttcatgtccgggaaatctacatggatcagcaatgagtatgatggtcaatatggagaaaaagaaagagtaattaccaattttttttcaattcaaaaatgtagatgtccgcagcgttattataaaatgaaagtacattttgataaaacgacaaattacgatccgtcgtatttataggcgaaagcaataaacaaattattctaattcggaaatctttatttcgacgtgtctacattcacgtccaaatgggggcttagatgagaaacttcacgatcgatgcggccgctctagaattcgagctctccggagtcgactttcacctttggacaatcattgacagtaacacaacgaacattaactgcatcgtaaaataaaatgtcactacaatttgataaaatattgtgtaaaagagagtacttaaaaaaaaatggcgtagaaagtaccatactcctattagctttcaacaagaaatagggaaagaaagatgactaaaacataaataaagaaagataggaaattgagccatattgttttcctttcttagatgtttcatgtggatattattttacttgatccttgtggaatttatgttatgctattattaactcttgtgtataaatagaatacaataccaatacttaaaatagacataaaaaacgatgtgagacaatagataattaaaccattcttacataatttattccttacaaaatataattcacagaatttacttttacataaaacagaacgcacacacaacacacttcacaatattctcaacctaaaaacagagtaatagagcttgaatatgtgtttaattaatcaaatctgggtaactggtctaacgggtcttggaggtgctggaagttcgaaatcccgttgccagaatccaacgtcgtgccatcctccgtgtttgtacccggcggccctcaatgttccgcgtgccgtgtagcctagtgcttcatgtaggcgcacagaaggatcgttaggtagtccgatgacggccaccactgacttaaagccctgagcctccatagatttgagtaagtgggtgtacagggtgctgcccagacccagcctctgatggcgatgcgagacgtaaacagtggattccacagtccagtcgtaagcatttcgagccttccacggtccagcgtaggcgattcccgcgacgacgccttcgacctctgccacgagccacggatagcggtcttgtagtctctccaagtcatcaatccattcctgaggagtttgtggttcggtacgaaagtttactgtggatgtctcgatgtagtgatttacgatgtcacatactgccgccatgtcggcggctgttgcgggtctaatctctactggccgtcgctcgggagacatggtaccaggttgagtagtgcagcacaaagtaaaacgtttaagatttgttgttgtaagccaagaaatgggtggtttggtattgatgcgattttcgctatatataatacagaaatctaaatactgtcttatcttgaaatatcaatctctgaggttgctattggctagtcatagattttttttggggtttagattgtttgtttgcttacgtggttaatggctcgcacttcgcagattcgcaattgatatgtgttttagatatctaggtgcgtttcatggattggcgaaacacccactcatgacgtgacacgtatacgaatagatctctctatccttatgaaatcctgagagcatcaatgtaaaccaaaacaaaaaaaatttggataattgccttgtataatacattttattaaaccttttgtatatatgcttcttttttttttcttcttctgataataataataactcttggctagagataaaacataagaacaaagtagataaaactataaaatttatgtatggaaataagattatctaaaactacggaattgaatcaaactttaccacagaaacataagaaattattaccatcttaattagcacctgtattgaccataatattttattagaagaagaagaaaacattggaatccaccagatcaaggtgatggtcagggtcataagacagctccataatcacgagtcatggtcatagactcatagccacaagtctgcttctcccttaccaataccaacggcttcaacaacggccacaactaataagccaattccaacggccaccatataactgaaccggatattcaccaaacccatttcaaccggaaattgatccacaagtaattaaaaatgttctgaccaaatgtggaataggtgggaaaatgttgaagtaaaggccaattctataatgtgagaggaaaactcttttttgagcttcaggaatattttcttagacctgaccttgaatctatattttgtagttcttcaatgatttgatttctctattcatttccttataaattaaattacttgtgaaatgaatttctgtttagaaaaaccttgaacttcctagttgaccaaaggattagaaaatctttctaagtggagtgtctcctaataatggatcaaaagtgtttggagcaaaaaagaacgcatagtgtattagtgttaacagcttttatagttcaaagtttagaatgtgctctaaagcatatgtgtgagcctgtgaggtctggtgattgtaaaaaagaaaaattgtgcttcgtggaaagtgatgcaataaacaacacttctagggctgagtaaaaccaactagacatgtaatgatagcttaaaccgttttctataggagtgtgattgaggagtaatttgttgatggatcccaagttgtttttccaagtcataaatcatttcccagtgattataaaaagcagagcagcttatccaaactcttgatttgtactataaactttcatgaaaagattgtactttcaaatggtaaagactaaaatttcaagattgtaaatgagtgaagaaacaaggttttataatgttttttaattatatatattgtgcagatgaacattgcattaacggttttgtttcatttactttatcccatttatcgtcatttataaaagatatagacacaatattattttataaaaaagtttggatttttaatcacgccacgattttttcagaacttcaatatcaattttgaatcaattttcaacattaacgattttttggataattataaaaaataacaaacatatatctcttagagatttattactgcataaacacacaaatacactttctagtaaccaaaatagtacaaattcaaagccatgaaatttatgagcttgattattcatgcactagttcacgccgcttctagttgttcgagcttctcaatctccctggataccctgacggctgcctcgtcgcaggacaacatggcgggtctgatcccgttcgcttcgacgtaagccctccgcctttcgatagcttcaacgaccactttgtcctccttcaccagggcttgagcttgccagctcctgagaacaccgtccatctctggatcgtctataccgaagttgcgggaactaccgaagaagtaatggcagcttgcctcggtctcaggagtaaggatatgggttcctcgggagtggattgactgctccttcggcgtcccttccggcgcgaccgcgatgaagttcagcatagccgacaccttattccaccggatgtcgttccaggcgtcaactggtgtgttagcaccacgcaagaacttagccatgagaactgagggtgtgccaccagggatcttcatgagcgcctggatctcgccgtcgccaacgatcacctcacgctcaagtctatcgaaggcgtctgtttgggcgttagcgcggtgcacgtactgagcgtgtccaagatccatgaggttatccacaaggagcttgtagttgcagtccacgtgaccgtaacctccgacagtacggtaagctggatcaacacgacacccaaagtcagggatagcaccgggatctgcgagtgctggatctccaggccagatccagatcaatgcgtctctttccacgacagggaatgatcgcacgttcagagaagcagggcgggctccgttgccgtgcgggttgtggacacactggccacctccgtcaaattccagaccgtgatatggacactggagatgtccgttgactagaataccgtcactcaacggagcgaagcgatgcggacaaatatcaagcagggcagcgacaactccgtcaggttgacgatagagagctagtggagtatcgaggatagttcttcctagaggcttctcgctcaactcctcaggaagtgcagcaacgtaccaagcgtttctaacgaaagtgagcatggcccgggaatctctcgtcaatggtggcaaataggaaagagtctcaaacttcttctttccaattggaggccacacctgcatgcactttactcttccaccattgcttgtaatggaagtaatgtcagtgttgaccttcctcactgggaatccagtcatggatttgaggccgccgaatggagccattgcggcggattgccccctagaggcacggctgactgttgtcacagcggaagaggatatcatagaagccatggtgcgatcgccacctgtcatgaccccaattaaacaaaaacaatttaacaattgaagatagatcgcgcaatcaagaatactctggaaaaacgttactcagatagcaaatcatatttcctacaatgcaatttaaatctgaaagacgtccgtcgaagaacataattctagtaaccagattcaatacatgtataaaaaaccaagaaccatggggaatcgataaattacaaatcttgagaagaaaaaaagataaaaatcatgataatcacaacatagaaagaagatgacaaaaaccaaatgtgaatcatccgaacaacgtcggacatcacgattaaaaatcgacctacctaacgaacagaaatgaaaaatacaacaaagacttcaaatcgtttctcgaaaattcaaacatcaattaatcgaacaggagggggaaaagatgcaatctgaaaacagaagatagagaaaagatgaaaaccttgagaggttgaagaagagcgaattacgaaataggaataaagcgcgatgagaaagtttagagtatttcgagtctgtatggaaaattagcgctcgagaattcacgtatttatacctaaaattgccgacttccgttaatcacgaataactataatattctataggaaaatgaaacgtgtctttgattacttgcgtgttacgtattgtttagttctggaagaatctagttggaaaggtcaattttccatttatacgattacgcgcagttgggaaagctttatggggaaagcataaagattaggaagttagagcagctcaattagctttattcacctctcaaatgggaaaaaggaaggaaaaaacaatacgattccttttattttctttttcttttttctaatttttctattaataaatttcatttgaatttagattttctgttcttaattttgatgtttgtatgtatttggttcaataaaattttgtatttgacaagtctaacaaatttgaaacctataaaaattaattaaatcctttatatattagtttgcattttgtgtcataaaatattagatgctatctttcacttccatagtttttcatgtaaaacctagattttttgtatgtgtatcaattaacacattcttcaaactttgaaaaatagcatacaaaatatattattttaattaaacttctaaattcattttattaagattacatatgaaaatagttcatggattcaattattacactttataggagtttaattgataacactataagaattgcttattatccaatgaatggggtaaatacaaaatttcaaattttggtaggcacgtgttaatccaaagatactaatctcggtttcaacaacagtaacctctcgatcttaactagctcaagatatgcaaccgaaaggaaatataaaaggcataaatgatacctaatctcttgacaagcagatttctagagcttagtaagaaaatatttatggagattacaaggaatagcaaaagaacataacaaatttattgtggttcgactaatagtgcctacatccactttgaagccgccatagagcttttattcaacttattcaaggatcaaacaagtatagagaatttgaaaatgagttttcttcctctctctctttaactttttctttttctttttctcattctatatttcatattgagttaaatggtttattatttacattacagagttatttaatggcagtaaatataacaaactctaactaacttattagtttttctatttgatttcagttgtaggatcacgaagctcaaaatctgtatttaggggtaccgagttgtcctgagaagtaattgcttcttcatcgagtagacagggaacaaagcatcaggtgcgtcgagattgaaccgagattgagccccacagtacaacttgcttagcttgttgaagagttagggcactgggattgtatcaagatctctcgagcttcaccaggttcttaagcaatagagtttcgaatcgagatctttcgagtttcaccgagattttctttaagtaataaaattcactcttctttttaacaatcctccaccttgaattttattgctaccaatgactatttcttctgattctattcttttaagtagtctattaccttgagcaactgtaggcagtgctcgagtttagcattagtaactattttagtcaacatatctgctgcattttcttttgtctgaacctttgatactgttacttttctctttgctatgacttctctaatgacatatagcttcacatcaatatgcttagttctttcgtgatattgcagatttttagacaagtgtattgcactttaactatcacacatgatgtttactgactgttaaaagaagccaagttcttcaagtagtccttttagccagacgacttctttaacaacttcagtcatggctataaactttacttttgttgtgaatagtgccactacaggctataggttaaacttccataaaatcacatttcctcttaggataaaagtaaaagcagcgagtgatcttcttttgtccaggtcttctgcataaccatacaactctagctttgtagacttagtatgcttgtagagtagccccttgctaggtgttcctttcagatgtggcgcgccggcaagtctttcgacccttgttcgtcactgctgacgtctttttctccattattggaggatgagtcactgcaagctgtaaagcttgtattattgctagtggaggccccacctgcacatgccgagcaattatgattgtcggccagtagctttttgagctgtttgcagagcaataacttagtggatgtccccaccattacatcgttattgatgatgctttcttcaaaggaagataagatgctgacatcctgtctggagattgtcgctccatagtgactcgtgatcaatactctttcatgtgagtaattgatttttatctttgtttctccgttgatccgcggtggatcgaccatgacattcaaaaacctttaagatatggatcttataaaataaatgtaaagggtaaacaattcttccttgcttaacccaagctatatattttatgcactaatttaggtattagagtatattcagctgaacaccacctaccaatgctagtactttaatcagtcaattctaacttcgataatatatctcaaccaaattagtgaaaaagagtcgtaaatgaaaaactatgtaccaagatattctatttgttttttttatgtttaaatatctcaaagataatacctaaaacgttttctcctcgtacaaagattcctcatttactttttattgtcgtaaactctaatacaataaactaaaacaagtacaaatacactagctttagaaatctactttttattgaaaccaaaaccaataattcaacatttcattttcaccgacaaacctttgtaaacaattgaagtaatttttgttggtactatgaatagtaacatcaagtcttcaagtgcatcatatcaaccaagacatgttcttaaaagcgacactaaaagatttaaaaccaaaagcatttatgaaatccgaacttaatcaaatcctaaatatttttcacttaaaaaaaaaaaaataggaagaaaaattgacataaatgggatattttcgttttcaaactggcaagccagcatgcaccacgttgttgacgtgtccttccacgtcggaaaaaaaaatattaccacagtaaaaagagaataaaatgaaagtcgttgactctcccttagtcggaggaagcgcgtgaagctgaagccggattagaaatcggcaataaccccgacacgtcatcgaaatgctagtatcaaatattgtccgttggatcttccttcaccaactctatttgaacggccacgatcttccaggtccaacggttcggaagaatcttttcgaaattccatggctagtccctacactcctccctattggctccctagggcatcccgaccggttattccggttgccgggaaggtggctggacgctataaatacccgctttgttcatctcgtagtccttgtaccgttgagcttcgccttctaatagagctctggttcggttggcgtattagctcgaattctttctctcttccagatctacgctgccgatttcatcaggtttgcgagctctgttccaccatttttcttttcctgaagctttgagcatgcttgtgattcttcatttcctcatttctttgatggtttatgaaagaatttaggggaattttctctttttgtattctagtggtactggtagatttgtttgaagtttgtttctcttcttctgagaagtgaattcttccagatctgacagttgcttttgattttttctttgggaattagtgaatgatacttcgatactgttttttgctctctgagattctggatctcgggccttggggttttctattgtcttttggtagctatgtttcgtttgtcagcttgtatttgtcattgttgaatggttcgatccggtttgtaaataaaataaattttgtaggcgcacttgttttccacggttttcgtgttacggtttcatgattccctagatctctggttagaactaagttttttgtcggtaattggatttggtaagggactgttactgtggttgaattgtagatccagtcatcttctacatgagtgtagggttccttagggcagatcttgtgttttataattttaattttgttgtttccctgattttgaacctgtttggttgttcagattcgtcgagtcatttccattcattaaaagtttctataattttatttgaatcttctgaatctgtgcttgtattacccagatttctataaacctatcttgatttcaagtgtgctatgtggtaactgttgatattttcaagcttaagcaatactgatgtgactaaaacttaactaatgaactgaatgttttttgtacacgaactaatatggtgttttgttatgtttcagaggacgcgtaccatggcgcaagttagcagaatctgcaatggtgtgcagaacccatctcttatctccaatctctcgaaatccagtcaacgcaaatctcccttatcggtttctctgaagacgcagcagcatccacgagcttatccgatttcgtcgtcgtggggattgaagaagagtgggatgacgttaattggctctgagcttcgtcctcttaaggtcatgtcttctgtttccacggcgtgcatgcttcacggtgcaagcagccgtccagcaactgctcgtaagtcctctggtctttctggaaccgtccgtattccaggtgacaagtctatctcccacaggtccttcatgtttggaggtctcgctagcggtgaaactcgtatcaccggtcttttggaaggtgaagatgttatcaacactggtaaggctatgcaagctatgggtgccagaatccgtaaggaaggtgatacttggatcattgatggtgttggtaacggtggactccttgctcctgaggctcctctcgatttcggtaacgctgcaactggttgccgtttgactatgggtcttgttggtgtttacgatttcgatagcactttcattggtgacgcttctctcactaagcgtccaatgggtcgtgtgttgaacccacttcgcgaaatgggtgtgcaggtgaagtctgaagacggtgatcgtcttccagttaccttgcgtggaccaaagactccaacgccaatcacctacagggtacctatggcttccgctcaagtgaagtccgctgttctgcttgctggtctcaacaccccaggtatcaccactgttatcgagccaatcatgactcgtgaccacactgaaaagatgcttcaaggttttggtgctaaccttaccgttgagactgatgctgacggtgtgcgtaccatccgtcttgaaggtcgtggtaagctcaccggtcaagtgattgatgttccaggtgatccatcctctactgctttcccattggttgctgccttgcttgttccaggttccgacgtcaccatccttaacgttttgatgaacccaacccgtactggtctcatcttgactctgcaggaaatgggtgccgacatcgaagtgatcaacccacgtcttgctggtggagaagacgtggctgacttgcgtgttcgttcttctactttgaagggtgttactgttccagaagaccgtgctccttctatgatcgacgagtatccaattctcgctgttgcagctgcattcgctgaaggtgctaccgttatgaacggtttggaagaactccgtgttaaggaaagcgaccgtctttctgctgtcgcaaacggtctcaagctcaacggtgttgattgcgatgaaggtgagacttctctcgtcgtgcgtggtcgtcctgacggtaagggtctcggtaacgcttctggagcagctgtcgctacccacctcgatcaccgtatcgctatgagcttcctcgttatgggtctcgtttctgaaaaccctgttactgttgatgatgctactatgatcgctactagcttcccagagttcatggatttgatggctggtcttggagctaagatcgaactctccgacactaaggctgcttgaaggcctttaatcatctgaaactgttcaccatgcatgcaatcttgtgaaatatatggttttaattagacttcaatcttatgttggctattgtactaataaaagcatgtcatgttattttcatttgattttatctgtactttggtttgtttgaagaataaagatgagcttgctatgcatgcatgcatgccatcgattatcagggtttccttttttcttttctggcttcccatcaatttggtgtgaattagtgtgtgtgatatattatattatgctatttatgaaataaattgttggttatatttgatctacaatctacatacatgtgatttttatcaacaaaatatctcgggaaacaatacctttttggtagcaaaattcaaataatactattttaaataaatcaaagttaaccaataccttattcaagttggaggggtctcaaacaagcaaaagaattcaagttgttaatgaacttcggttaatgataaaagaattcgcatttaaaacctgcaggtccggagagctcatttaaataagcttgatggggatcagattgtcgtttcccgccttcagtttaaactatcagtgtttcatgaagaggtatgtgcacgaagttcaagaattggtaggaattcgttgaagaaaaaaaagagaaaaggaaggggacctacaaagtcaatagggaagcataccctaatgactttggagttggtaaaaccttgtggaaagtggcggcataagtatggcatgcaaattggtattgatataaaaattggtcttggcatgcaaattggtctttgcatgcaaaaacttcccattgtacactcatgaaaagaaatacccgagggcttaaagaagtgtctatgggagcatactatggtaatgttcacgtacaataacgtttacattcttcattatttagttatatatgagtgtaatattaatgatatcatataaattacaacaattgcggaaactaatatgtgaaatgcgttttattgtagaatcttttcaatatcgaagatgatgaagcaaagaagagcctatttctatatgccgttgccgagagattcagagatttcaaggctaagttagtgtccgggtggatcactaagcggaggaatgttgtaggtacaaagaagaagaaaatatacggcatccggttatcacgcttcaccgaaagaatcaaccgagccttccaaattaccatttcatatttggaagattatcaaacctacagagtgggcggcatttgttgccaaaaacactacaccacaagctattgtaagaatattatactaacatttaactacagttaactcaaatgtttttgtattaacctttgtttgtaaattgtgttttaggcaagacgtgaaaaaagtagtgcaagcacaaaagctaggaagtattatcatcatttgggtgcgcattcgtacaatgaggcgagagaggatgttcgcccattaatccacaatggggatgacaacggcaacaaagactagctcaaccaccacagctacacagactcgacatccagataaagtcctcgatttgtggtgtttgatgcatagggttgatgcatagggaaaaa

SEQ ID NO: 11 - DNA, 1000 bp, Beta vulgaris
  source 1..1000: mol_type = genomic DNA
aaacatgataatatgaggtattatataaatagtgtgtcaatttttaggtttgcattaattgttggtcgaatttcgagaaattaaatgtgattttaggcctatttcggccttcttccatccaggggtgagggagggatgtggtggggaggggagcttggcaggggtgtgggaggtggtatgtgcaaggaggtggggaggtgggcaggggtgtggggtgggaaggggtgtgagatgtttgtgggtggccaggggcgtagagttgggaggtgtgcaaaggtggggatatgtgggcaggtgtgggttagggaggtgtggggtggggaggtgtgggtgtggagaggtgggtatgggtgtgtgtaaggtgggaacaggtagtctgtgcacctgtacgcaccctacacatacctacacccacctccccacacccacacccctacaccttcttacccctcgcgcacccacctccccaccccctgcataccccttctgcacgccttgtacacctcccccacccctcccccacccctcccccaacctcttccccaaccctacccaccctcccccaacccttccccacccctcccccacctttgtccacctcccctaacccccttcccacccatcccccacccctgcccacctcccacaaacccttatccaccctccctgcccatccctgcccacctcccccaactccctgcccacccctcccctacccctacccctacccctaccccttgtcctttagtgcggcttgtaattttttccttgagctatatatattaatgtatatttcgtataatttcagggagatggttgatttgcaaaaggcgaggagctcgggaattaaaaacactaccgaggacgagtcatatgatgagagaaaggaacaatataaaggtgatatggaggatcgagatcttgatggtgacttcgttactagttttgagatcctagtggatgaaaatgctctaatagaaaaggattacatgtaaggccg

SEQ ID NO: 12 - DNA, 1000 bp, Beta vulgaris
  source 1..1000: mol_type = genomic DNA
catgaagaggtatgtgcacgaagttcaagaattggtaggaattcgttgaagaaaaaaaagagaaaaggaaggggacctacaaagtcaatagggaagcataccctaatgactttggagttggtaaaaccttgtggaaagtggcggcataagtatggcatgcaaattggtattgatataaaaattggtcttggcatgcaaattggtctttgcatgcaaaaacttcccattgtacactcatgaaaagaaatacccgagggcttaaagaagtgtctatgggagcatactatggtaatgttcacgtacaataacgtttacattcttcattatttagttatatatgagtgtaatattaatgatatcatataaattacaacaattgcggaaactaatatgtgaaatgcgttttattgtagaatcttttcaatatcgaagatgatgaagcaaagaagagcctatttctatatgccgttgccgagagattcagagatttcaaggctaagttagtgtccgggtggatcactaagcggaggaatgttgtaggtacaaagaagaagaaaatatacggcatccggttatcacgcttcaccgaaagaatcaaccgagccttccaaattaccatttcatatttggaagattatcaaacctacagagtgggcggcatttgttgccaaaaacactacaccacaagctattgtaagaatattatactaacatttaactacagttaactcaaatgtttttgtattaacctttgtttgtaaattgtgttttaggcaagacgtgaaaaaagtagtgcaagcacaaaagctaggaagtattatcatcatttgggtgcgcattcgtacaatgaggcgagagaggatgttcgcccattaatccacaatggggatgacaacggcaacaaagactagctcaaccaccacagctacacagactcgacatccagataaagtcctcgatttgtggtgtttgatgcatagggttgatgcatagggaaaaa

SEQ ID NO: 13 - DNA, 2007 bp, Beta vulgaris
  source 1..2007: mol_type = genomic DNA
aaacatgataatatgaggtattatataaatagtgtgtcaatttttaggtttgcattaattgttggtcgaatttcgagaaattaaatgtgattttaggcctatttcggccttcttccatccaggggtgagggagggatgtggtggggaggggagcttggcaggggtgtgggaggtggtatgtgcaaggaggtggggaggtgggcaggggtgtggggtgggaaggggtgtgagatgtttgtgggtggccaggggcgtagagttgggaggtgtgcaaaggtggggatatgtgggcaggtgtgggttagggaggtgtggggtggggaggtgtgggtgtggagaggtgggtatgggtgtgtgtaaggtgggaacaggtagtctgtgcacctgtacgcaccctacacatacctacacccacctccccacacccacacccctacaccttcttacccctcgcgcacccacctccccaccccctgcataccccttctgcacgccttgtacacctcccccacccctcccccacccctcccccaacctcttccccaaccctacccaccctcccccaacccttccccacccctcccccacctttgtccacctcccctaacccccttcccacccatcccccacccctgcccacctcccacaaacccttatccaccctccctgcccatccctgcccacctcccccaactccctgcccacccctcccctacccctacccctacccctaccccttgtcctttagtgcggcttgtaattttttccttgagctatatatattaatgtatatttcgtataatttcagggagatggttgatttgcaaaaggcgaggagctcgggaattaaaaacactaccgaggacgagtcatatgatgagagaaaggaacaatataaaggtgatatggaggatcgagatcttgatggtgacttcgttactagttttgagatcctagtggatgaaaatgctctaatagaaaaggattacatgtaaggccgacctcgccatgaagaggtatgtgcacgaagttcaagaattggtaggaattcgttgaagaaaaaaaagagaaaaggaaggggacctacaaagtcaatagggaagcataccctaatgactttggagttggtaaaaccttgtggaaagtggcggcataagtatggcatgcaaattggtattgatataaaaattggtcttggcatgcaaattggtctttgcatgcaaaaacttcccattgtacactcatgaaaagaaatacccgagggcttaaagaagtgtctatgggagcatactatggtaatgttcacgtacaataacgtttacattcttcattatttagttatatatgagtgtaatattaatgatatcatataaattacaacaattgcggaaactaatatgtgaaatgcgttttattgtagaatcttttcaatatcgaagatgatgaagcaaagaagagcctatttctatatgccgttgccgagagattcagagatttcaaggctaagttagtgtccgggtggatcactaagcggaggaatgttgtaggtacaaagaagaagaaaatatacggcatccggttatcacgcttcaccgaaagaatcaaccgagccttccaaattaccatttcatatttggaagattatcaaacctacagagtgggcggcatttgttgccaaaaacactacaccacaagctattgtaagaatattatactaacatttaactacagttaactcaaatgtttttgtattaacctttgtttgtaaattgtgttttaggcaagacgtgaaaaaagtagtgcaagcacaaaagctaggaagtattatcatcatttgggtgcgcattcgtacaatgaggcgagagaggatgttcgcccattaatccacaatggggatgacaacggcaacaaagactagctcaaccaccacagctacacagactcgacatccagataaagtcctcgatttgtggtgtttgatgcatagggttgatgcatagggaaaaa

SEQ ID NO: 14 - DNA, 47 bp, synthetic construct
  source 1..47: mol_type = other DNA; note = primer
gaaggtgaccaagttcatgctaatagaaaaggattacatgtaaggcc

SEQ ID NO: 15 - DNA, 26 bp, synthetic construct
  source 1..26: mol_type = other DNA; note = primer
aatagaaaaggattacatgtaaggcc

SEQ ID NO: 16 - DNA, 41 bp, synthetic construct
  source 1..41: mol_type = other DNA; note = primer
gaaggtgaccaagttcatgctacgcacaccgtcagcatcag

SEQ ID NO: 17 - DNA, 27 bp, synthetic construct
  source 1..27: mol_type = other DNA; note = primer
caaggttttggtgctaaccttaccgtt

SEQ ID NO: 18 - DNA, 26 bp, synthetic construct
  source 1..26: mol_type = other DNA; note = primer
ctgatccatgtagatttcccggacat

SEQ ID NO: 19 - DNA, 48 bp, synthetic construct
  source 1..48: mol_type = other DNA; note = primer
gaaggtgaccaagttcatgctgccttcagtttaaactatcagtgtttc

SEQ ID NO: 20 - DNA, 27 bp, synthetic construct
  source 1..27: mol_type = other DNA; note = primer
gccttcagtttaaactatcagtgtttc

SEQ ID NO: 21 - DNA, 43 bp, synthetic construct
  source 1..43: mol_type = other DNA; note = primer
gaaggtgaccaagttcatgctgaggctcagggctttaagtcag

SEQ ID NO: 22 - DNA, 25 bp, synthetic construct
  source 1..25: mol_type = other DNA; note = primer
cagaaggatcgttaggtagtccgat

SEQ ID NO: 23 - DNA, 29 bp, synthetic construct
  source 1..29: mol_type = other DNA; note = primer
aattcttgaacttcgtgcacatacctctt

SEQ ID NO: 24 - DNA, 44 bp, synthetic construct
  source 1..44: mol_type = other DNA; note = primer
gaaggtgaccaagttcatgctcacatacctcttcatggcgaggt

SEQ ID NO: 25 - DNA, 45 bp, synthetic construct
  source 1..45: mol_type = other DNA; note = primer
gaaggtcggagtcaacggattccatgtagatttcccggacatgaa

SEQ ID NO: 26 - DNA, 30 bp, synthetic construct
  source 1..30: mol_type = other DNA; note = primer
gtggatgaaaatgctctaatagaaaaggat

SEQ ID NO: 27 - DNA, 42 bp, synthetic construct
  source 1..42: mol_type = other DNA; note = primer
gaaggtgaccaagttcatgcttacatgtaaggccgacctcgc

SEQ ID NO: 28 - DNA, 48 bp, synthetic construct
  source 1..48: mol_type = other DNA; note = primer
gaaggtcggagtcaacggattcgccttcagtttaaactatcagtgttt

SEQ ID NO: 29 - DNA, 30 bp, synthetic construct
  source 1..30: mol_type = other DNA; note = primer
ttcaacgaattcctaccaattcttgaactt

SEQ ID NO: 30 - DNA, 23 bp, synthetic construct
  source 1..23: mol_type = other DNA; note = primer
cacatacctcttcatggcgaggt

SEQ ID NO: 31 - DNA, 24 bp, synthetic construct
  source 1..24: mol_type = other DNA; note = primer
ccatgtagatttcccggacatgaa

SEQ ID NO: 32 - DNA, 21 bp, synthetic construct
  source 1..21: mol_type = other DNA; note = primer
tacatgtaaggccgacctcgc

SEQ ID NO: 33 - DNA, 27 bp, synthetic construct
  source 1..27: mol_type = other DNA; note = primer
cgccttcagtttaaactatcagtgttt

SEQ ID NO: 34 - DNA, 17 bp, synthetic construct
  source 1..17: mol_type = other DNA; note = primer
tgtcgtttcccgccttc

SEQ ID NO: 35 - DNA, 24 bp, synthetic construct
  source 1..24: mol_type = other DNA; note = primer
tcctaccaattcttgaacttcgtg

SEQ ID NO: 36 - DNA, 16 bp, synthetic construct
  source 1..16: mol_type = other DNA; note = probe
actatcagtgtttcat

SEQ ID NO: 37 - DNA, 40 bp, synthetic construct
  source 1..40: mol_type = other DNA; note = primer
gaaggtgaccaagttcatgctggcccgggaatctctcgtc

SEQ ID NO: 38 - DNA, 29 bp, synthetic construct
  source 1..29: mol_type = other DNA; note = primer
tttgagactctttcctatttgccaccatt

SEQ ID NO: 39 - DNA, 39 bp, synthetic construct
  source 1..39: mol_type = other DNA; note = primer
gaaggtgaccaagttcatgctgccactcgaccgccggct

SEQ ID NO: 40 - DNA, 25 bp, synthetic construct
  source 1..25: mol_type = other DNA; note = primer
acggagtgatccacatgggactttt

SEQ ID NO: 41 - DNA, 40 bp, synthetic construct
  source 1..40: mol_type = other DNA; note = primer
gaaggtgaccaagttcatgctccgtcgtcaggctttccga

SEQ ID NO: 42 - DNA, 28 bp, synthetic construct
  source 1..28: mol_type = other DNA; note = primer
gataatgacttctgttcaaccacccaaa

SEQ ID NO: 43 - DNA, 42 bp, synthetic construct
  source 1..42: mol_type = other DNA; note = primer
gaaggtgaccaagttcatgctcgatattcccgttccgccgga

SEQ ID NO: 44 - DNA, 23 bp, synthetic construct
  source 1..23: mol_type = other DNA; note = primer
ctccgtaacccagctttgggcaa

SEQ ID NO: 45 - DNA, 24 bp, synthetic construct
  source 1..24: mol_type = other DNA; note = primer
gacctccatattactgaaaggaag

SEQ ID NO: 46 - DNA, 23 bp, synthetic construct
  source 1..23: mol_type = other DNA; note = primer
gagtaattgctccatcctgttca

SEQ ID NO: 47 - DNA, 27 bp, synthetic construct
  source 1..27: mol_type = other DNA; note = probe
ctacgaagtttaaagtatgtgccgctc

SEQ ID NO: 48 - DNA, 442 bp, Agrobacterium tumefaciens
  source 1..442: mol_type = genomic DNA
ctcatctaagcccccatttggacgtgaatgtagacacgtcgaaataaagatttccgaattagaataatttgtttattgctttcgcctataaatacgacggatcgtaatttgtcgttttatcaaaatgtactttcattttataataacgctgcggacatctacatttttgaattgaaaaaaaattggtaattactctttctttttctccatattgaccatcatactcattgctgatccatgtagatttcccggacatgaagccatttacaattgaatatatcctgccgccgctgccgctttgcacccggtggagcttgcatgttggtttctacgcagaactgagccggttaggcagataatttccattgagaactgagccatgtgcaccttccccccaacacggtgagcgacggggcaacggagtgatccacatgggactttt

SEQ ID NO: 49 - DNA, 52 bp, synthetic construct
  source 1..52: mol_type = other DNA; note = intervening sequence
aaacttcacgatcgatgcggccgctctagaattcgagctctccggagtcgac

SEQ ID NO: 50 - DNA, 500 bp, Medicago truncatula
  source 1..500: mol_type = genomic DNA
acacatattcaagctctattactctgtttttaggttgagaatattgtgaagtgtgttgtgtgtgcgttctgttttatgtaaaagtaaattctgtgaattatattttgtaaggaataaattatgtaagaatggtttaattatctattgtctcacatcgttttttatgtctattttaagtattggtattgtattctatttatacacaagagttaataatagcataacataaattccacaaggatcaagtaaaataatatccacatgaaacatctaagaaaggaaaacaatatggctcaatttcctatctttctttatttatgttttagtcatctttctttccctatttcttgttgaaagctaataggagtatggtactttctacgccatttttttttaagtactctcttttacacaatattttatcaaattgtagtgacattttattttacgatgcagttaatgttcgttgtgttactgtcaatgattgtccaaaggtgaaa

SEQ ID NO: 51 - skipped

SEQ ID NO: 52 - DNA, 552 bp, synthetic construct
  source 1..552: mol_type = other DNA; note = codon optimized for plant
atgtctcccgagcgacggccagtagagattagacccgcaacagccgccgacatggcggcagtatgtgacatcgtaaatcactacatcgagacatccacagtaaactttcgtaccgaaccacaaactcctcaggaatggattgatgacttggagagactacaagaccgctatccgtggctcgtggcagaggtcgaaggcgtcgtcgcgggaatcgcctacgctggaccgtggaaggctcgaaatgcttacgactggactgtggaatccactgtttacgtctcgcatcgccatcagaggctgggtctgggcagcaccctgtacacccacttactcaaatctatggaggctcagggctttaagtcagtggtggccgtcatcggactacctaacgatccttctgtgcgcctacatgaagcactaggctacacggcacgcggaacattgagggccgccgggtacaaacacggaggatggcacgacgttggattctggcaacgggatttcgaacttccagcacctccaagacccgttagaccagttacccagatttga

SEQ ID NO: 53 - skipped

SEQ ID NO: 54 - DNA, 70 bp, Arabidopsis thaliana
  source 1..70: mol_type = genomic DNA
caaaccacccatttcttggcttacaacaacaaatcttaaacgttttactttgtgctgcactactcaacct

SEQ ID NO: 55 - DNA, 1318 bp, Arabidopsis thaliana
  source 1..1318: mol_type = genomic DNA
atcaacaaattactcctcaatcacactcctatagaaaacggtttaagctatcattacatgtctagttggttttactcagccctagaagtgttgtttattgcatcactttccacgaagcacaatttttcttttttacaatcaccagacctcacaggctcacacatatgctttagagcacattctaaactttgaactataaaagctgttaacactaatacactatgcgttcttttttgctccaaacacttttgatccattattaggagacactccacttagaaagattttctaatcctttggtcaactaggaagttcaaggtttttctaaacagaaattcatttcacaagtaatttaatttataaggaaatgaatagagaaatcaaatcattgaagaactacaaaatatagattcaaggtcaggtctaagaaaatattcctgaagctcaaaaaagagttttcctctcacattatagaattggcctttacttcaacattttcccacctattccacatttggtcagaacatttttaattacttgtggatcaatttccggttgaaatgggtttggtgaatatccggttcagttatatggtggccgttggaattggcttattagttgtggccgttgttgaagccgttggtattggtaagggagaagcagacttgtggctatgagtctatgaccatgactcgtgattatggagctgtcttatgaccctgaccatcaccttgatctggtggattccaatgttttcttcttcttctaataaaatattatggtcaatacaggtgctaattaagatggtaataatttcttatgtttctgtggtaaagtttgattcaattccgtagttttagataatcttatttccatacataaattttatagttttatctactttgttcttatgttttatctctagccaagagttattattattatcagaagaagaaaaaaaaaagaagcatatatacaaaaggtttaataaaatgtattatacaaggcaattatccaaattttttttgttttggtttacattgatgctctcaggatttcataaggatagagagatctattcgtatacgtgtcacgtcatgagtgggtgtttcgccaatccatgaaacgcacctagatatctaaaacacatatcaattgcgaatctgcgaagtgcgagccattaaccacgtaagcaaacaaacaatctaaaccccaaaaaaaatctatgactagccaatagcaacctcagagattgatatttcaagataagacagtatttagatttctgtattatatatagcgaaaatcgcatcaatac

SEQ ID NO: 56 - skipped

SEQ ID NO: 57 - DNA, 500 bp, Medicago truncatula
  source 1..500: mol_type = genomic DNA
gcatgaataatcaagctcataaatttcatggctttgaatttgtactattttggttactagaaagtgtatttgtgtgtttatgcagtaataaatctctaagagatatatgtttgttattttttataattatccaaaaaatcgttaatgttgaaaattgattcaaaattgatattgaagttctgaaaaaatcgtggcgtgattaaaaatccaaacttttttataaaataatattgtgtctatatcttttataaatgacgataaatgggataaagtaaatgaaacaaaaccgttaatgcaatgttcatctgcacaatatatataattaaaaaacattataaaaccttgtttcttcactcatttacaatcttgaaattttagtctttaccatttgaaagtacaatcttttcatgaaagtttatagtacaaatcaagagtttggataagctgctctgctttttataatcactgggaaatgatttatgacttggaaaaacaacttg

SEQ ID NO: 58 - skipped

SEQ ID NO: 59 - DNA, 1023 bp, synthetic construct
  source 1..1023: mol_type = other DNA; note = codon optimized for plant
atgctcactttcgttagaaacgcttggtacgttgctgcacttcctgaggagttgagcgagaagcctctaggaagaactatcctcgatactccactagctctctatcgtcaacctgacggagttgtcgctgccctgcttgatatttgtccgcatcgcttcgctccgttgagtgacggtattctagtcaacggacatctccagtgtccatatcacggtctggaatttgacggaggtggccagtgtgtccacaacccgcacggcaacggagcccgccctgcttctctgaacgtgcgatcattccctgtcgtggaaagagacgcattgatctggatctggcctggagatccagcactcgcagatcccggtgctatccctgactttgggtgtcgtgttgatccagcttaccgtactgtcggaggttacggtcacgtggactgcaactacaagctccttgtggataacctcatggatcttggacacgctcagtacgtgcaccgcgctaacgcccaaacagacgccttcgatagacttgagcgtgaggtgatcgttggcgacggcgagatccaggcgctcatgaagatccctggtggcacaccctcagttctcatggctaagttcttgcgtggtgctaacacaccagttgacgcctggaacgacatccggtggaataaggtgtcggctatgctgaacttcatcgcggtcgcgccggaagggacgccgaaggagcagtcaatccactcccgaggaacccatatccttactcctgagaccgaggcaagctgccattacttcttcggtagttcccgcaacttcggtatagacgatccagagatggacggtgttctcaggagctggcaagctcaagccctggtgaaggaggacaaagtggtcgttgaagctatcgaaaggcggagggcttacgtcgaagcgaacgggatcagacccgccatgttgtcctgcgacgaggcagccgtcagggtatccagggagattgagaagctcgaacaactagaagcggcgtga

SEQ ID NO: 60 - DNA, 252 bp, Pisum sativum
  source 1..252: mol_type = genomic DNA
atggcttctatgatatcctcttccgctgtgacaacagtcagccgtgcctctagggggcaatccgccgcaatggctccattcggcggcctcaaatccatgactggattcccagtgaggaaggtcaacactgacattacttccattacaagcaatggtggaagagtaaagtgcatgcaggtgtggcctccaattggaaagaagaagtttgagactctttcctatttgccaccattgacgagagattcccgggcc

SEQ ID NO: 61 - DNA, 11 bp, synthetic construct
  source 1..11: mol_type = other DNA; note = intervening sequence
ggtgcgatcgc

SEQ ID NO: 62 - DNA, 461 bp, Cucumis melo
  source 1..461: mol_type = genomic DNA
gttttcatcttttctctatcttctgttttcagattgcatcttttccccctcctgttcgattaattgatgtttgaattttcgagaaacgatttgaagtctttgttgtatttttcatttctgttcgttaggtaggtcgatttttaatcgtgatgtccgacgttgttcggatgattcacatttggtttttgtcatcttctttctatgttgtgattatcatgatttttatctttttttcttctcaagatttgtaatttatcgattccccatggttcttggttttttatacatgtattgaatctggttactagaattatgttcttcgacggacgtctttcagatttaaattgcattgtaggaaatatgatttgctatctgagtaacgtttttccagagtattcttgattgcgcgatctatcttcaattgttaaattgtttttgtttaattggggtcatgacaggtg

SEQ ID NO: 63 - DNA, 82 bp, Cucumis melo
  source 1..82: mol_type = genomic DNA
tccatacagactcgaaatactctaaactttctcatcgcgctttattcctatttcgtaattcgctcttcttcaacctctcaag

SEQ ID NO: 64 - DNA, 2068 bp, Cucumis melo
  source 1..2068: mol_type = genomic DNA
atctgaaaggaacacctagcaaggggctactctacaagcatactaagtctacaaagctagagttgtatggttatgcagaagacctggacaaaagaagatcactcgctgcttttacttttatcctaagaggaaatgtgattttatggaagtttaacctatagcctgtagtggcactattcacaacaaaagtaaagtttatagccatgactgaagttgttaaagaagtcgtctggctaaaaggactacttgaagaacttggcttcttttaacagtcagtaaacatcatgtgtgatagttaaagtgcaatacacttgtctaaaaatctgcaatatcacgaaagaactaagcatattgatgtgaagctatatgtcattagagaagtcatagcaaagagaaaagtaacagtatcaaaggttcagacaaaagaaaatgcagcagatatgttgactaaaatagttactaatgctaaactcgagcactgcctacagttgctcaaggtaatagactacttaaaagaatagaatcagaagaaatagtcattggtagcaataaaattcaaggtggaggattgttaaaaagaagagtgaattttattacttaaagaaaatctcggtgaaactcgaaagatctcgattcgaaactctattgcttaagaacctggtgaagctcgagagatcttgatacaatcccagtgccctaactcttcaacaagctaagcaagttgtactgtggggctcaatctcggttcaatctcgacgcacctgatgctttgttccctgtctactcgatgaagaagcaattacttctcaggacaactcggtacccctaaatacagattttgagcttcgtgatcctacaactgaaatcaaatagaaaaactaataagttagttagagtttgttatatttactgccattaaataactctgtaatgtaaataataaaccatttaactcaatatgaaatatagaatgagaaaaagaaaaagaaaaagttaaagagagagaggaagaaaactcattttcaaattctctatacttgtttgatccttgaataagttgaataaaagctctatggcggcttcaaagtggatgtaggcactattagtcgaaccacaataaatttgttatgttcttttgctattccttgtaatctccataaatattttcttactaagctctagaaatctgcttgtcaagagattaggtatcatttatgccttttatatttcctttcggttgcatatcttgagctagttaagatcgagaggttactgttgttgaaaccgagattagtatctttggattaacacgtgcctaccaaaatttgaaattttgtatttaccccattcattggataataagcaattcttatagtgttatcaattaaactcctataaagtgtaataattgaatccatgaactattttcatatgtaatcttaataaaatgaatttagaagtttaattaaaataatatattttgtatgctatttttcaaagtttgaagaatgtgttaattgatacacatacaaaaaatctaggttttacatgaaaaactatggaagtgaaagatagcatctaatattttatgacacaaaatgcaaactaatatataaaggatttaattaatttttataggtttcaaatttgttagacttgtcaaatacaaaattttattgaaccaaatacatacaaacatcaaaattaagaacagaaaatctaaattcaaatgaaatttattaatagaaaaattagaaaaaagaaaaagaaaataaaaggaatcgtattgttttttccttcctttttcccatttgagaggtgaataaagctaattgagctgctctaacttcctaatctttatgctttccccataaagctttcccaactgcgcgtaatcgtataaatggaaaattgacctttccaactagattcttccagaactaaacaatacgtaacacgcaagtaatcaaagacacgtttcattttcctatagaatattatagttattcgtgattaacggaagtcggcaattttaggtataaatacgtgaattctcgagcgctaattt

SEQ ID NO: 65 - DNA, 10 bp, synthetic construct
  source 1..10: mol_type = other DNA; note = intervening sequence
gtggcgcgcc

SEQ ID NO: 66 - DNA, 332 bp, Dahlia mosaic virus
  source 1..332: mol_type = genomic DNA
atcaacggagaaacaaagataaaaatcaattactcacatgaaagagtattgatcacgagtcactatggagcgacaatctccagacaggatgtcagcatcttatcttcctttgaagaaagcatcatcaataacgatgtaatggtggggacatccactaagttattgctctgcaaacagctcaaaaagctactggccgacaatcataattgctcggcatgtgcaggtggggcctccactagcaataatacaagctttacagcttgcagtgactcatcctccaataatggagaaaaagacgtcagcagtgacgaacaagggtcgaaagacttgcc

SEQ ID NO: 67 - skipped

SEQ ID NO: 68 - DNA, 1067 bp, Cucumis melo
  source 1..1067: mol_type = genomic DNA
tggatcgaccatgacattcaaaaacctttaagatatggatcttataaaataaatgtaaagggtaaacaattcttccttgcttaacccaagctatatattttatgcactaatttaggtattagagtatattcagctgaacaccacctaccaatgctagtactttaatcagtcaattctaacttcgataatatatctcaaccaaattagtgaaaaagagtcgtaaatgaaaaactatgtaccaagatattctatttgttttttttatgtttaaatatctcaaagataatacctaaaacgttttctcctcgtacaaagattcctcatttactttttattgtcgtaaactctaatacaataaactaaaacaagtacaaatacactagctttagaaatctactttttattgaaaccaaaaccaataattcaacatttcattttcaccgacaaacctttgtaaacaattgaagtaatttttgttggtactatgaatagtaacatcaagtcttcaagtgcatcatatcaaccaagacatgttcttaaaagcgacactaaaagatttaaaaccaaaagcatttatgaaatccgaacttaatcaaatcctaaatatttttcacttaaaaaaaaaaaaataggaagaaaaattgacataaatgggatattttcgttttcaaactggcaagccagcatgcaccacgttgttgacgtgtccttccacgtcggaaaaaaaaatattaccacagtaaaaagagaataaaatgaaagtcgttgactctcccttagtcggaggaagcgcgtgaagctgaagccggattagaaatcggcaataaccccgacacgtcatcgaaatgctagtatcaaatattgtccgttggatcttccttcaccaactctatttgaacggccacgatcttccaggtccaacggttcggaagaatcttttcgaaattccatggctagtccctacactcctccctattggctccctagggcatcccgaccggttattccggttgccgggaaggtggctggacgctataaatacccgctttgttcatctcgtagtcctt

SEQ ID NO: 69 - DNA, 92 bp, Cucumis melo
  source 1..92: mol_type = genomic DNA
gtaccgttgagcttcgccttctaatagagctctggttcggttggcgtattagctcgaattctttctctcttccagatctacgctgccgattt

SEQ ID NO: 70 - DNA, 845 bp, Cucumis melo
  source 1..845: mol_type = genomic DNA
catcaggtttgcgagctctgttccaccatttttcttttcctgaagctttgagcatgcttgtgattcttcatttcctcatttctttgatggtttatgaaagaatttaggggaattttctctttttgtattctagtggtactggtagatttgtttgaagtttgtttctcttcttctgagaagtgaattcttccagatctgacagttgcttttgattttttctttgggaattagtgaatgatacttcgatactgttttttgctctctgagattctggatctcgggccttggggttttctattgtcttttggtagctatgtttcgtttgtcagcttgtatttgtcattgttgaatggttcgatccggtttgtaaataaaataaattttgtaggcgcacttgttttccacggttttcgtgttacggtttcatgattccctagatctctggttagaactaagttttttgtcggtaattggatttggtaagggactgttactgtggttgaattgtagatccagtcatcttctacatgagtgtagggttccttagggcagatcttgtgttttataattttaattttgttgtttccctgattttgaacctgtttggttgttcagattcgtcgagtcatttccattcattaaaagtttctataattttatttgaatcttctgaatctgtgcttgtattacccagatttctataaacctatcttgatttcaagtgtgctatgtggtaactgttgatattttcaagcttaagcaatactgatgtgactaaaacttaactaatgaactgaatgttttttgtacacgaactaatatggtgttttgttatgtttcagagg

SEQ ID NO: 71 - skipped

SEQ ID NO: 72 - DNA, 228 bp, Arabidopsis thaliana
  source 1..228: mol_type = genomic DNA
atggcgcaagttagcagaatctgcaatggtgtgcagaacccatctcttatctccaatctctcgaaatccagtcaacgcaaatctcccttatcggtttctctgaagacgcagcagcatccacgagcttatccgatttcgtcgtcgtggggattgaagaagagtgggatgacgttaattggctctgagcttcgtcctcttaaggtcatgtcttctgtttccacggcgtgc

SEQ ID NO: 73 - DNA, 1368 bp, synthetic construct
  source 1..1368: mol_type = other DNA; note = codon optimized for plant
atgcttcacggtgcaagcagccgtccagcaactgctcgtaagtcctctggtctttctggaaccgtccgtattccaggtgacaagtctatctcccacaggtccttcatgtttggaggtctcgctagcggtgaaactcgtatcaccggtcttttggaaggtgaagatgttatcaacactggtaaggctatgcaagctatgggtgccagaatccgtaaggaaggtgatacttggatcattgatggtgttggtaacggtggactccttgctcctgaggctcctctcgatttcggtaacgctgcaactggttgccgtttgactatgggtcttgttggtgtttacgatttcgatagcactttcattggtgacgcttctctcactaagcgtccaatgggtcgtgtgttgaacccacttcgcgaaatgggtgtgcaggtgaagtctgaagacggtgatcgtcttccagttaccttgcgtggaccaaagactccaacgccaatcacctacagggtacctatggcttccgctcaagtgaagtccgctgttctgcttgctggtctcaacaccccaggtatcaccactgttatcgagccaatcatgactcgtgaccacactgaaaagatgcttcaaggttttggtgctaaccttaccgttgagactgatgctgacggtgtgcgtaccatccgtcttgaaggtcgtggtaagctcaccggtcaagtgattgatgttccaggtgatccatcctctactgctttcccattggttgctgccttgcttgttccaggttccgacgtcaccatccttaacgttttgatgaacccaacccgtactggtctcatcttgactctgcaggaaatgggtgccgacatcgaagtgatcaacccacgtcttgctggtggagaagacgtggctgacttgcgtgttcgttcttctactttgaagggtgttactgttccagaagaccgtgctccttctatgatcgacgagtatccaattctcgctgttgcagctgcattcgctgaaggtgctaccgttatgaacggtttggaagaactccgtgttaaggaaagcgaccgtctttctgctgtcgcaaacggtctcaagctcaacggtgttgattgcgatgaaggtgagacttctctcgtcgtgcgtggtcgtcctgacggtaagggtctcggtaacgcttctggagcagctgtcgctacccacctcgatcaccgtatcgctatgagcttcctcgttatgggtctcgtttctgaaaaccctgttactgttgatgatgctactatgatcgctactagcttcccagagttcatggatttgatggctggtcttggagctaagatcgaactctccgacactaaggctgcttga

SEQ ID NO: 74 - skipped

SEQ ID NO: 75 - DNA, 500 bp, Medicago truncatula
  source 1..500: mol_type = genomic DNA
ttaatcatctgaaactgttcaccatgcatgcaatcttgtgaaatatatggttttaattagacttcaatcttatgttggctattgtactaataaaagcatgtcatgttattttcatttgattttatctgtactttggtttgtttgaagaataaagatgagcttgctatgcatgcatgcatgccatcgattatcagggtttccttttttcttttctggcttcccatcaatttggtgtgaattagtgtgtgtgatatattatattatgctatttatgaaataaattgttggttatatttgatctacaatctacatacatgtgatttttatcaacaaaatatctcgggaaacaatacctttttggtagcaaaattcaaataatactattttaaataaatcaaagttaaccaataccttattcaagttggaggggtctcaaacaagcaaaagaattcaagttgttaatgaacttcggttaatgataaaagaattcgcatttaaaa

SEQ ID NO: 76 - DNA, 44 bp, synthetic construct
  source 1..44: mol_type = other DNA; note = intervening sequence
cctgcaggtccggagagctcatttaaataagcttgatggggatc

SEQ ID NO: 77 - DNA, 357 bp, Agrobacterium tumefaciens
  source 1..357: mol_type = genomic DNA
aggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgcacggaatgccaagcactcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatct

(19) United States
(12) Patent Application Publication
(10) Pub. No.: US 2025/0295081 A1
(43) Pub. Date: Sep. 25, 2025

(54) TRANSGENIC SUGAR BEET EVENT BV_CSM63713 AND METHODS FOR DETECTION AND USES THEREOF

(71) Applicant: Monsanto Technology LLC, St. Louis, MO (US)

(72) Inventors: Karen Lynn Fincher, Pacific, MO (US); Maik Gertz, Hannover (DE); Margarita Mantai, Einbeck (DE); Paloma Moe, Prior Lake, MN (US); Oscar C. Sparks, Jr., Florissant, MO (US); Zach Stumo, Salt Lake City, UT (US); David Wurbs, Einbeck (DE)

(21) Appl. No.: 18/863,562

(22) PCT Filed: May 5, 2023
(86) PCT No.: PCT/US2023/066651

§ 371 Date: Nov. 6, 2024

Related U.S. Application Data
(60) Provisional application No. 63/340,278, filed on May 10, 2022.

(51) Int. Cl. A01H 1/04; A01H 1/00; C12Q 1/6895

(52) U.S. Cl. CPC A01H 1/045; A01H 1/123; C12Q 1/6895; C12Q 2600/13

(57) ABSTRACT

A transgenic sugar beet event, Bv_CSM63713, is provided. Transgenic plant cells, plant parts, plants, seeds, agricultural and commodity products containing event Bv_CSM63713 are also provided. Recombinant DNA molecules unique to the event Bv_CSM63713, and methods of using and detecting Bv_CSM63713 are also provided. Sugar beet plants containing the event Bv_CSM63713 exhibit tolerance to benzoic acid auxins such as dicamba; inhibitors of EPSPS such as glyphosate; and inhibitors of glutamine synthetase such as glufosinate.

### REFERENCE TO RELATED APPLICATIONS

This application claims the benefit of U.S. provisional application No. 63/340,278, filed May 10, 2022, herein incorporated by reference in its entirety.

### INCORPORATION OF SEQUENCE LISTING

The sequence listing contained in the file named “MONS531WO_ST26.xml”, which is 113 kilobytes (measured in MS-Windows), was created on Apr. 5, 2023, is filed herewith by electronic submission, and is incorporated by reference herein in its entirety.

### FIELD OF THE INVENTION

The present disclosure relates generally to the fields of agriculture, plant biotechnology and molecular biology. More specifically, the disclosure relates to compositions and methods for providing herbicide tolerance in transgenic sugar beet plants. More specifically, recombinant DNA molecules of sugar beet event Bv_CSM63713 are provided. Also provided are transgenic sugar beet plants, plant parts, seeds, cells, and agricultural products comprising the sugar beet event Bv_CSM63713, as well as methods of using transgenic sugar beet plants, plant parts, seeds, cells, and agricultural products comprising the sugar beet event Bv_CSM63713, methods of detecting sugar beet event Bv_CSM63713, and methods of controlling weeds. Transgenic sugar beet plants, plant parts, seeds and cells comprising sugar beet event Bv_CSM63713 exhibit tolerance to benzoic acid auxins such as dicamba; inhibitors of 5-enolpyruvylshikimate-3-phosphate synthase (EPSPS) such as glyphosate; and inhibitors of glutamine synthetase such as glufosinate.

### BACKGROUND OF THE INVENTION

Sugar beet (Beta vulgaris) is an important commercial crop in many countries, and the important role of herbicides for weed control in crop production is well-established. Weeds compete with crops for space, nutrients, water, and light and can contaminate harvests, thus making weed control essential to obtaining a successful crop yield. Biotechnological methods have been shown to be useful in production of transgenic sugar beets tolerant to a specific herbicide through expression of a heterologous gene (a transgene). Transgenic herbicide tolerance enables the use of an herbicide in a crop growing environment without crop injury or with minimal crop injury (e.g., less than about 10% injury). Transgenic traits in sugar beets have been used to impart tolerance to glyphosate and are used broadly in commercial sugar beet production for weed control.

Transgenic glyphosate tolerance is produced by inserting into the genome of the sugar beet the capability to produce a variant of the glyphosate target, the enzyme 5-enolpyruvyl-3-phophoshikimic acid synthase (EPSPS). This variant, which is glyphosate tolerant, is the CP4-EPSPS from Agrobacterium sp. strain CP4.

An herbicide tolerance trait can be used alone or combined with other traits, such as tolerance to another herbicide. Combinations of herbicide tolerance traits are desirable to provide weed control options that increase grower flexibility and enable the use of multiple herbicide modes of action for controlling challenging weeds. Combining multiple desired traits in the genome can be achieved by making crosses between two parents each having a desired trait, and identifying progeny plants that have combinations of the desired traits, or by retransforming a transgenic plant comprising one or more desired trait(s) with one or more genes for additional desired traits, either through random integration or through targeted integration of the one or more genes for additional desired traits. Alternatively, combining multiple desired traits can be achieved by inserting multiple genes as a single DNA molecule into one location, or locus, in the genome. The combination of multiple herbicide tolerance traits at a single locus in sugar beet would provide a useful tool in weed control that is much simpler and less expensive to maintain during subsequent breeding to form hybrids with a diverse pool of elite germplasms.

The expression of a transgene in a transgenic plant, part, seed, or cell, and therefore its effectiveness, may be influenced by many factors, such as the regulatory elements used in the transgene's expression cassette, the combination and/or interaction of these regulatory elements, the chromosomal location of the transgene insertion site, the chromatin structure of the genome at or near the transgene insertion site, and the presence or proximity of any endogenous cis and/or trans regulatory elements or genes close to the transgene insertion site. In addition, the performance of the trait in the transgenic plant is further complicated when the transgenic insert comprises multiple expression cassettes, each having a different transgene conferring a distinct trait. These differences or factors may result in variation in the level of transgene expression or in the spatial or temporal pattern of transgene expression among different transgenic insertion events of the same expression cassettes. Furthermore, different transgenic events can also vary in terms of the molecular quality of the events. For example, a transgenic event may contain two or more copies of the transgene insertion at one or more chromosomal locations, or a transgenic insertion may be truncated relative to the intended insertion or contain vector backbone sequences, or a transgene may be inserted into an endogenous gene or in a repeated region. Such characteristics may result in undesirable outcomes, such as gene silencing, altered pattern and/or expression of the transgene, altered pattern and/or expression of the endogenous genes. There may also be undesirable phenotypic or agronomic differences among different events.

Even in the case of targeted sequence insertion, variability in the level of transgene expression between independent but genetically identical targeted sequence insertion (TSI) events was observed in a subset of transgenic events (Verkest et al., 2019). This expression variability and silencing occurred independently of the transgene sequence and could be attributed to DNA methylation that was further linked to different DNA methylation mechanisms. The fact that a considerable variation in transgene expression was observed in a subset of clean TSI events shows that even when integration events are targeted, selection remains necessary similarly to the practice for random integration events in order to identify TSI events with stable gene of interest expression over generations.

A commercially useful multi-gene transgenic event requires that each of the transgenes in the transgenic insert express in the manner necessary for that trait to be successful, and involves rigorous testing, evaluation, and selection. Once a tolerance trait has been chosen, individual expression cassettes are designed and tested in vitro and/or in planta to select for the best expression cassettes for each trait. Such tests include testing different regulatory elements (e.g., promoters, introns, leaders, and 3′ UTRs) and combinations of different regulatory elements for desirable spatial and temporal expression of the transgenes, as well as examining whether to target the product of the transgenes (proteins) to subcellular compartments such as chloroplasts. Then the selected expression cassettes for each trait are combined into one construct, and the construct is tested to ensure that all the expression cassettes function well together and each transgene is properly expressed. The selected combinations of expression cassettes are then used for transformation to produce transgenic plants. Since Agrobacterium-mediated transformation with a T-DNA construct comprising one or more transgene cassettes is largely variable and random in terms of where the transgene(s) can be inserted into the plant genome, each transgenic event is unique with random and unique insertion of the transgenic DNA in a different plant genomic location. Thus, the selected combinations of expression cassettes are used to produce hundreds of unique multi-gene transgenic events, each the result of a random insertion of the foreign DNA in a different plant genomic location.

For these reasons, the performance of different transformation events from the same transformation construct can vary, and the identification of transformation events conferring the most beneficial traits or characteristics without other potential off-types or concerns is needed to select a superior event for commercial use. Therefore, a large number of individual transgenic events must be produced and analyzed to select an event having superior commercial properties, which can be a significant undertaking that involves analysis and selection among many different transformation events.

To establish a multi-gene event for commercial use requires rigorous molecular characterization, greenhouse testing, and field trials in different germplasms over multiple years, in multiple locations and under a variety of conditions, allowing extensive agronomic, phenotypic, and molecular data to be obtained. The resulting data are then analyzed to select an event that is suitable for commercial purposes. The commercial multi-gene event, once identified as having the desired transgene expression, molecular characteristics, efficacy and field performance, can then be introgressed as a single locus having multiple herbicide tolerance traits into other sugar beet genetic backgrounds using plant breeding methods. The resulting sugar beet varieties contain the new traits combined with other desirable qualities such as native traits, disease tolerance traits, high-yielding germplasm, and/or one or more other transgenic herbicide tolerance traits.

### SUMMARY OF THE INVENTION

Recombinant DNA molecules are provided herein. Examples of such recombinant DNA molecules include a sequence selected from the group consisting of SEQ ID NO:10, SEQ ID NO:1, SEQ ID NO:2, SEQ ID NO:3, SEQ ID NO:4, SEQ ID NO:5, SEQ ID NO:6, SEQ ID NO:7, SEQ ID NO:8, and SEQ ID NO:9, a polynucleotide having a nucleotide sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or to the full length of SEQ ID NO: 9, and a complete complement of any of the foregoing. In some embodiments, the recombinant DNA molecule is derived from a plant, seed, plant part, plant cell, progeny plant, or commodity product comprising sugar beet event Bv_CSM63713, a representative sample of seed comprising the event having been deposited as ATCC Accession No. PTA-127098. In some embodiments, the recombinant DNA molecule is comprised in a plant, seed, plant part, plant cell, or progeny plant comprising sugar beet event Bv_CSM63713, or a commodity product produced therefrom, a representative sample of seed comprising the event having been deposited as ATCC Accession No. PTA-127098. The recombinant DNA molecule can be formed by the insertion of a heterologous nucleic acid molecule into the genomic DNA of a sugar beet plant or sugar beet cell. The recombinant DNA molecule can comprise an amplicon diagnostic for the presence of sugar beet event Bv_CSM63713.

DNA molecules that function as DNA probes are provided. An example of such a DNA molecule is a DNA molecule comprising a polynucleotide segment of sufficient length to function as a DNA probe that hybridizes specifically under stringent hybridization conditions with sugar beet event Bv_CSM63713 DNA in a sample. Detecting hybridization of the DNA molecule under the stringent hybridization conditions is diagnostic for the presence of sugar beet event Bv_CSM63713 in the sample.

Also provided is a DNA molecule comprising a polynucleotide segment of sufficient length to function as a DNA probe specific for detecting in a sample at least one of: a 5′ junction sequence between flanking sugar beet genomic DNA and the transgenic insert of sugar beet event Bv_CSM63713; a 3′ junction sequence between the transgenic insert of sugar beet event Bv_CSM63713 and flanking sugar beet genomic DNA; SEQ ID NO:9; and a fragment of SEQ ID NO:9 comprising a sufficient length of contiguous nucleotides of SEQ ID NO:9 to identify the sequence as a fragment of the transgenic insert of Bv_CSM63713.

The DNA probe can comprise SEQ ID NO:36. Alternatively, the DNA molecule that functions as a DNA probe can comprise a nucleotide sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, and a complement of any of the foregoing. The sample can be derived from a sugar beet plant, seed, plant part, plant cell, progeny plant, or commodity product.

A pair of DNA molecules is provided. The pair of DNA molecules comprises a first DNA molecule and a second DNA molecule. The first and the second DNA molecules comprise a fragment of SEQ ID NO:10 or a complement thereof and function as DNA primers when used together in an amplification reaction with DNA comprising sugar beet event Bv_CSM63713 to produce an amplicon diagnostic for sugar beet event Bv_CSM63713 in a sample. For example, the first and second DNA molecules can comprise SEQ ID NO:14 and SEQ ID NO:18; SEQ ID NO:15 and SEQ ID NO:18; SEQ ID NO:19 and SEQ ID NO:23; SEQ ID NO:20 and SEQ ID NO:23; SEQ ID NO:25 and SEQ ID NO:26; SEQ ID NO: 31 and SEQ ID NO: 26; SEQ ID NO: 33 and SEQ ID NO: 29; SEQ ID NO:28 and SEQ ID NO:29; or SEQ ID NO:34 and SEQ ID NO:35. The amplicon can comprise a nucleotide sequence selected from the group consisting of: SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; SEQ ID NO:10; and a fragment of any of SEQ ID NO:1, SEQ ID NO:2, SEQ ID NO:3, SEQ ID NO:4, SEQ ID NO:5, SEQ ID NO:6, SEQ ID NO:7, SEQ ID NO:8, wherein the fragment is at least 10 nucleotides in length and comprises nucleotides 1,000-1,001 or 12,722-12,723 of SEQ ID NO:10.

Methods for detecting the presence of sugar beet event Bv_CSM63713 in a sample derived from a sugar beet seed, plant, plant part, plant cell, progeny plant, or commodity product are provided. In a first example of such a method, the method comprises: a) contacting the sample with any of the DNA molecules that function as probes described herein; b) subjecting the sample and the DNA molecule that functions as a probe to stringent hybridization conditions; and c) detecting the hybridization of the DNA molecule that functions as a probe to a DNA molecule in the sample. The hybridization of the DNA molecule that functions as a probe to the DNA molecule in the sample is diagnostic for the presence of sugar beet event Bv_CSM63713 in the sample.

Another method of detecting the presence of sugar beet event Bv_CSM63713 in a sample derived from a sugar beet seed, plant, plant part, plant cell, progeny plant, or commodity product is provided. The method comprises: contacting the sample with any of the pairs of DNA molecules described herein; b) performing an amplification reaction sufficient to produce a DNA amplicon; and c) detecting the presence of the DNA amplicon. The DNA amplicon comprises at least one of: a 5′ junction sequence between flanking sugar beet genomic DNA and the transgenic insert of sugar beet event Bv_CSM63713; a 3′ junction sequence between flanking sugar beet genomic DNA and the transgenic insert of sugar beet event Bv_CSM63713; SEQ ID NO:9; and a fragment of SEQ ID NO:9 comprising a sufficient length of contiguous nucleotides of SEQ ID NO:9 to identify the sequence as a fragment of the transgenic insert of Bv_CSM63713. The presence of the DNA amplicon indicates the presence of sugar beet event Bv_CSM63713 in the sample. The DNA amplicon can be at least 10 nucleotides in length, at least 11 nucleotides in length, at least 12 nucleotides in length, at least 13 nucleotides in length, at least 14 nucleotides in length, at least 15 nucleotides in length, at least 16 nucleotides in length, at least 17 nucleotides in length, at least 18 nucleotides in length, at least 19 nucleotides in length, at least 20 nucleotides in length, at least 25 nucleotides in length, at least 30 nucleotides in length, at least 35 nucleotides in length, at least 40 nucleotides in length, at least 45 nucleotides in length, at least 50 nucleotides in length, at least 60 nucleotides in length, at least 70 nucleotides in length, at least 80 nucleotides in length, at least 90 nucleotides in length, or at least 100 nucleotides in length. The DNA amplicon can comprise a nucleotide sequence selected from the group consisting of SEQ ID NO:10; SEQ ID NO:9; SEQ ID NO:8; SEQ ID NO:7; SEQ ID NO:6; SEQ ID NO:5; SEQ ID NO:4; SEQ ID NO:3; SEQ ID NO:2 and SEQ ID NO:1; and a fragment of any of SEQ ID NO:10, SEQ ID NO:8, SEQ ID NO:7, SEQ ID NO:6, SEQ ID NO:5, SEQ ID NO:4, SEQ ID NO:3, SEQ ID NO:2, and SEQ ID NO:1 that is at least 10 nucleotides in length and comprises nucleotides 1,000-1,001 or 12,722-12,723 of SEQ ID NO:10.

Another method of detecting the presence of sugar beet event Bv_CSM63713 in a sample of DNA derived from a sugar beet seed, plant, plant part, plant cell, progeny plant or commodity product is provided. The method comprises: a) contacting the sample with any of the DNA molecules that function as probes described herein; and b) performing a sequencing reaction to produce a target sequence. The target sequence comprises a nucleotide sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, a complete complement of any thereof, and a fragment of any of SEQ ID NO:1, SEQ ID NO:2, SEQ ID NO:3, SEQ ID NO:4, SEQ ID NO:5, SEQ ID NO:6, SEQ ID NO:7, SEQ ID NO:8, and SEQ ID NO:10 that is at least 10 nucleotides long and comprises nucleotides 1,000-1,001 or 12,722-12,723 of SEQ ID NO:10.

A further method of detecting the presence of sugar beet event Bv_CSM63713 in a sample derived from a sugar beet seed, plant, plant part, plant cell, progeny plant, or commodity product is provided. The method comprises: a) contacting the sample with at least one antibody specific for at least one protein encoded by sugar beet event Bv_CSM63713; and b) detecting binding of the antibody to the protein in the sample. The binding of the antibody to the protein indicates the presence of sugar beet event Bv_CSM63713 in the sample.

DNA detection kits for detecting the presence of sugar beet event Bv_CSM63713 in a sample are provided. One example of such a DNA detection kit is a kit comprising any of the pairs of DNA primers described herein. Another example of a DNA detection kit is a kit comprising any of the DNA molecules that function as a probe described herein.

Also provided are protein detection kits for detecting the presence of sugar beet event Bv_CSM63713 in a sample. One example of such a kit is a kit comprising at least one antibody specific for at least one protein encoded by sugar beet event Bv_CSM63713. Detecting binding of the at least one antibody to the at least one protein encoded by sugar beet event Bv_CSM63713 in a sample is diagnostic for the presence of sugar beet event Bv_CSM63713 in the sample.

A sugar beet seed, plant, plant part, or plant cell is provided. The sugar beet seed, plant, plant part, or plant cell comprises a recombinant DNA molecule comprising a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10, a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or the full length of SEQ ID NO: 9, and a complete complement of any of the foregoing. The sugar beet seed, plant, plant part or plant cell can express at least one herbicide tolerance gene selected from the group consisting of phosphinothricin N-acetyltransferase (PAT), dicamba monooxygenase (DMO), 5-enolpyruvylshikimate-3-phosphate synthase (EPSPS), and any combination thereof. The sugar beet seed, plant, plant part or plant cell can be tolerant to at least one herbicide selected from the group consisting of glufosinate, dicamba, glyphosate, and any combination thereof. The sugar beet plant, plant seed, plant part, or plant cell can comprise sugar beet event Bv_CSM63713, a representative sample of seed comprising the event having been deposited under ATCC Accession No. PTA-127098. The sugar beet plant, plant seed, plant part, or plant cell can be further defined as a progeny plant of any generation of a sugar beet plant comprising sugar beet event Bv_CSM63713, or a sugar beet plant part, plant seed, or plant cell derived therefrom.

Further provided are sugar beet plants, plant parts, plant seeds, and plant cells. The sugar beet plants, plant parts, plant seeds, and plant cells comprise sugar beet event Bv_CSM63713, a representative sample of seed comprising sugar beet event Bv_CSM63713 having been deposited under ATCC Accession No. PTA-127098.

Any of the sugar beet plant parts described herein can comprise a root, a beet, a microspore, pollen, an anther, an ovule, an ovary, a flower, an embryo, a stem, a leaf, a protoplast, or a callus.

Methods for controlling or preventing weeds in an area are provided. One example of such a method comprises planting sugar beet comprising event Bv_CSM63713 in the area, and applying an effective amount of at least one herbicide selected from the group consisting of dicamba, glyphosate, glufosinate and any combination thereof, to control the weeds in the area without injury to the sugar beet or with less than about 10% injury to the sugar beet. Applying the effective amount of at least one herbicide can comprise applying at least two or more herbicides selected from the group consisting of dicamba, glyphosate, glufosinate, and any combination thereof over a growing season. The effective amount of dicamba can be about 0.5 lb ae/acre to about 2 lb ae/acre of dicamba over a growing season. The effective amount of glufosinate can be about 0.4 lb ai/acre to about 2.16 lb ai/acre of glufosinate over a growing season. The effective amount of glyphosate can be about 0.75 lb ae/acre to about 2.25 lb ae/acre of glyphosate over a growing season.

Methods for controlling volunteer sugar beet comprising sugar beet event Bv_CSM63713 in an area are provided. One example of such a method comprises applying an herbicidally effective amount of at least one herbicide other than dicamba, glyphosate, or glufosinate, wherein the herbicide application prevents growth of sugar beet comprising sugar beet event Bv_CSM63713. The herbicide other than dicamba, glyphosate, or glufosinate can be selected from the group consisting of paraquat, clethodim, clopyralid, desmedipham, triflusulfuron, 2,4-dichlorophenoxyacetic acid (2, 4-D), and acetolactate synthase (ALS) inhibitors such as sulfonylureas (SUs), imidazolinones, triazolopyrimidines, pyrinidinyl oxybenzoates, and sulfonylamino carbonyl triazolinones.

Methods of obtaining a seed of a sugar beet plant or a sugar beet plant that is tolerant to glyphosate, dicamba, glufosinate, or any combination thereof are provided. In one example of such a method, the method comprises: a) obtaining a population of progeny seed or plants grown therefrom, at least one of which comprises sugar beet event Bv_CSM63713; and b) identifying at least a first progeny seed or plant grown therefrom that comprises sugar beet event Bv_CSM63713. Identifying the progeny seed or plant grown therefrom that comprises sugar beet event Bv_CSM63713 can comprise: a) growing the progeny seed or plant to produce progeny plants; b) treating the progeny plants with an effective amount of at least one herbicide selected from the group consisting of glyphosate, dicamba, glufosinate, and any combination thereof; and c) selecting a progeny plant that is tolerant to the at least one herbicide selected from the group consisting of glyphosate, dicamba, glufosinate, and any combination thereof. Alternatively or in addition, identifying progeny seed or plant grown therefrom that comprises sugar beet event Bv_CSM63713 can comprise detecting the presence of sugar beet event Bv_CSM63713 in a sample derived from the progeny seed or plant grown therefrom. Alternatively or in addition, identifying progeny seed or plant grown therefrom that comprises sugar beet event Bv_CSM63713 can comprise detecting the presence of at least one protein encoded by sugar beet event Bv_CSM63713 in a sample derived from the progeny seed or plant grown therefrom.

Methods of determining zygosity of a sugar beet plant, plant part, plant seed, or plant cell comprising sugar beet event Bv_CSM63713 are provided. One example of such a method comprises: a) contacting a sample comprising DNA derived from the sugar beet plant, plant part, plant seed, or plant cell with a primer set capable of producing a first amplicon diagnostic for the presence of sugar beet event Bv_CSM63713 and a second amplicon diagnostic for the wild-type sugar beet genomic DNA not comprising sugar beet event Bv_CSM63713; b) performing a nucleic acid amplification reaction; and c) detecting the first amplicon and the second amplicon. The presence of both amplicons indicates the sample is heterozygous for sugar beet event Bv_CSM63713, and the presence of only the first amplicon indicates the sample is homozygous for sugar beet event Bv_CSM63713. Illustrative examples of the primer sets that can be used are primer sets comprising SEQ ID NO:24, SEQ ID NO:25 and SEQ ID NO:26; SEQ ID NO:30, SEQ ID NO:31 and SEQ ID NO:26; SEQ ID NO:27, SEQ ID NO:28 and SEQ ID NO:29, and SEQ ID NO:32, SEQ ID NO:33 and SEQ ID NO:29.

Another method of determining the zygosity of a sugar beet plant, plant part, plant seed, or plant cell comprising sugar beet event Bv_CSM63713 is provided. The method comprises: a) contacting a sample comprising DNA derived from the sugar beet plant, plant part, plant seed, or plant cell with a probe set comprising at least a first probe that specifically hybridizes to sugar beet event Bv_CSM63713, and at least a second probe that specifically hybridizes to sugar genomic DNA that was disrupted by insertion of the heterologous DNA of sugar beet event Bv_CSM63713 but does not hybridize to sugar beet event Bv_CSM63713; and b) hybridizing the probe set with the sample under stringent hybridization conditions. Detecting hybridization of only the first probe under the hybridization conditions is diagnostic for a sugar beet plant, plant part, seed or plant cell homozygous for sugar beet event Bv_CSM63713. Detecting hybridization of both the first probe and the second probe under the hybridization conditions is diagnostic for a sugar plant, plant part, seed, or plant cell heterozygous for sugar beet event Bv_CSM63713.

DNA constructs are provided. One example of such a DNA construct comprises a first expression cassette, a second expression cassette, and a third expression cassette. The first expression cassette comprises in operable linkage i) a chlorophyll A-B binding protein (Cab1) promoter and leader from Arabidopsis thaliana, ii) a phosphinothricin N-acetyltransferase (PAT) coding sequence, and iii) a small heat shock protein (Hsp20) 3′ UTR from Medicago truncatula. The second expression cassette comprises in operable linkage i) a ubiquitin (Ubq1) promoter, leader and intron from Cucumis melo, ii) a ribulose bisphosphate carboxylase small subunit (RbcS) chloroplast transit peptide coding sequence from Pisum sativum, iii) a dicamba monooxygenase coding sequence (DMO), and iv) a putative protein 3′ UTR from Medicago truncatula. The third expression cassette comprises in operable linkage i) an inclusion body matrix protein enhancer from Dahlia Mosaic Virus, ii) an S-adenosyl-L-methionine synthetase (SAMS2) promoter, leader and intron from Cucumis melo, iii) a 5-enolpyruvylshikimate-3-phosphate synthase chloroplast transit peptide (EPSPS) coding sequence from Arabidopsis thaliana, iv) a 5-enolpyruvylshikimate-3-phosphate synthase (EPSPS) coding sequence, and v) a hypothetical protein 3′ UTR from Medicago truncatula. For example, the DNA construct can comprise SEQ ID NO:9.

Sugar beet plants, plant seeds, plant parts, or plant cells comprising any of the DNA constructs described herein are also provided.

A method of improving tolerance to at least one herbicide selected from the group consisting of glyphosate, dicamba, glufosinate, and any combination thereof in a sugar beet plant is provided. The method comprises a) inserting any of the DNA constructs described herein into the genome of a sugar beet cell; b) generating a sugar beet plant from the sugar beet cell; and c) selecting a sugar beet plant comprising the DNA construct. The selecting can comprise treating the sugar beet cell or plant with an effective amount of at least one herbicide selected from the group consisting of glyphosate, dicamba, glufosinate, and any combination thereof.

Also provided is a sugar beet plant, plant seed, plant part, or plant cell tolerant to herbicides with three different herbicide modes of action at a single genomic location. The sugar beet plant, plant seed, plant part or plant cell can comprise any of the DNA constructs described herein.

A sugar beet seed, plant, plant part, or plant cell tolerant to at least one herbicide selected from the group consisting of glufosinate, dicamba, glyphosate, and any combination thereof is provided. The sugar beet seed, plant, plant part, or plant cell comprises any of the DNA constructs provided herein.

Any of the sugar beet seeds, plants, plant parts, or cells can be obtained by any of the methods of improving tolerance to at least one herbicide selected from the group consisting of glufosinate, dicamba, glyphosate, and any combination thereof in a sugar beet plant provided herein.

A method of producing a progeny sugar beet plant comprising sugar beet event Bv_CSM63713 is provided. The method comprises: a) sexually crossing a first sugar beet plant that comprises sugar beet event Bv_CSM63713 with itself or a second sugar beet plant; b) collecting one or more seeds produced from the cross; c) growing one or more seeds to produce one or more progeny plants; and d) selecting at least a first progeny plant or seed comprising sugar beet event Bv_CSM63713. Inbred and hybrid sugar beet plants and seeds comprising sugar beet event Bv_CSM63713 that are produced by the method are also provided.

Nonliving sugar beet plant material and nonregenerable sugar beet plant material are also provided. The material can comprise any of the recombinant DNA molecules or any of the DNA constructs described herein.

Also provided is nonliving sugar beet plant material or nonregenerable sugar beet plant material comprising sugar beet event Bv_CSM63713, a representative sample of seed comprising the sugar beet event Bv_CSM63713 having been deposited under ATCC Accession No. PTA-127098.

Commodity products are also provided. An example of such a commodity product is a commodity product comprising any of the recombinant DNA molecules or any of the DNA constructs described herein. The commodity product can be produced from a transgenic sugar beet plant, plant part, plant seed, or plant cell comprising the sugar beet event Bv_CSM63713. The commodity product can comprise for example, whole or processed seeds, nonviable seeds, processed plant parts, processed plant tissues, dehydrated plant tissues, dehydrated plant parts, frozen plant tissues, frozen plant parts, plant parts processed for animal feed, fiber, pulp, pulp pellets, pulp shreds, tailings, juice, syrup, molasses, extract, raffinate, betaine, separator molasses solubles (SMS), or any other food for human consumption, viable seeds, viable plant parts (such as roots and leaves), or viable plant cells.

A method of producing a commodity product is provided. The method comprises: a) obtaining a transgenic sugar beet plant, plant part, or plant seed comprising sugar beet event Bv_CSM63713; and b) producing a commodity product from the transgenic sugar beet plant, plant part, or plant seed.

A method of controlling, preventing, or reducing the development of herbicide-tolerant weeds is provided. The method comprises cultivating in a crop growing environment a sugar beet plant comprising transgenes that provide tolerance to herbicides with three different herbicide modes of action at a single genomic location. The three different herbicide modes of action can be selected from the group consisting of inhibition of glutamine synthetase, benzoic acid auxins, and inhibition of EPSPS.

Also provided is a method for controlling, preventing, or reducing the development of herbicide-tolerant weeds. The method comprises: a) cultivating in a crop growing environment a sugar beet plant comprising any of the DNA constructs described herein for providing tolerance to herbicides with three different herbicide modes of action at a single genomic location; and b) applying to the crop growing environment at least one herbicide selected from the group consisting of glufosinate, dicamba, glyphosate, and any combination thereof, wherein the sugar beet plant is tolerant to the at least one herbicide.

A method of reducing loci for sugar beet breeding by inserting transgenes at a single genomic location for tolerance to three different classes of herbicides is provided. The transgenes can be inserted as a single molecularly linked transgenic insert. The single molecularly linked transgenic insert can provide a commercial level of tolerance to at least one herbicide for each herbicide mode of action.

Further provided are sugar beet plants, plant cells, plant parts, and plant seeds. The sugar beet plants, plant cells, plant parts, and plant seeds comprise a recombinant DNA construct integrated in chromosome 4. The recombinant DNA construct confers tolerance to at least one herbicide selected from the group consisting of glufosinate, dicamba, glyphosate, and combinations of any thereof. The recombinant DNA construct is integrated in a position of said chromosome flanked by SEQ ID NO:11 and SEQ ID NO:12.

### BRIEF DESCRIPTION OF THE DRAWINGS

FIG. 1 illustrates the sequence of sugar beet event Bv_CSM63713. Horizontal lines correspond to the positions of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:11, and SEQ ID NO:12 relative to SEQ ID NO:10. The horizontal arrows labeled SEQ ID NO:14/15 and SEQ ID NO:18, SEQ ID NO: 19/20 and SEQ ID NO:23, and SEQ ID NO:34 and SEQ ID NO:35 represent the approximate positions of three illustrative primer pairs that can be used to detect sugar beet event Bv_CSM63713. The horizontal bar without arrow labeled SEQ ID NO:36 represents the approximate position of an illustrative probe. The dotted horizontal arrows labeled SEQ ID NO:24/30, SEQ ID NO:25/31, and SEQ ID NO:26 represent the approximate positions of an illustrative primer set for determining the zygosity of a sugar beet plant comprising the event Bv_CSM63713. The hollow horizontal arrows labeled SEQ ID NO:27/32, SEQ ID NO:28/33 and SEQ ID NO:29 represent the approximate positions of another illustrative primer set for determining the zygosity of a sugar beet plant comprising the event Bv_CSM63713. Wherever applicable, when two numbers are separated by “/” following “SEQ ID NO:” the first number represents a SEQ ID NO for a primer with a labeled tail, whereas the second number after “/” represents a SEQ ID NO for the same primer, but without the labeled tail. “RB” refers to the Agrobacterium T-DNA right border; “LB” refers to the Agrobacterium T-DNA left border. “P” represents a promoter element; “L” represents a leader (5′ UTR) element; “I” represents an intron element; “CTP” represents a chloroplast transit peptide element; “T” represents a 3′ UTR. “PAT” represents a phosphinothricin N-acetyltransferase coding element; “DMO” represents a dicamba monooxygenase coding element; and “CP4” represents a 5-enolpyruvylshikimate-3-phosphate synthase coding element.

FIG. 2 provides photographs illustrating the effect of herbicide treatment on sugar beet. R0 plants grown in the greenhouse at the 4-6 leaf stage were treated with glyphosate (4×), glufosinate (4×), dicamba (4×), dicamba+glyphosate (4× each), and glyphosate+glufosinate+dicamba (2.4× each). Photos were taken before and two weeks after herbicide applications. Panels A1 and A2: wild-type sugar beet before and after herbicide application; Panels B1 and B2: event Bv_CSM63713 before and after herbicide application; Panels C1 and C2: another event before and after herbicide application. The herbicide treatments (from left to right) were: 1. glyphosate+glufosinate+dicamba triple mix; 2. glyphosate+dicamba double mix; 3. dicamba; 4. glufosinate; 5. glyphosate; and 6. untreated control.

### BRIEF DESCRIPTION OF THE SEQUENCES

SEQ ID NO:1 is a 30-nucleotide sequence representing the 5′ junction region of the sugar beet genomic DNA and the integrated transgene insert. SEQ ID NO:1 corresponds to nucleotide positions 986-1015 of SEQ ID NO:10.

SEQ ID NO:2 is a 30-nucleotide sequence representing the 3′ junction region of the integrated transgene insert and the sugar beet genomic DNA. SEQ ID NO:2 corresponds to nucleotide positions 12708-12737 of SEQ ID NO:10.

SEQ ID NO:3 is a 60-nucleotide sequence representing the 5′ junction region of the sugar beet genomic DNA and the integrated transgene insert. SEQ ID NO:3 corresponds to nucleotide positions 971-1030 of SEQ ID NO:10.

SEQ ID NO:4 is a 60-nucleotide sequence representing the 3′ junction region of the integrated transgene insert and the sugar beet genomic DNA. SEQ ID NO:4 corresponds to nucleotide positions 12693-12752 of SEQ ID NO:10.

SEQ ID NO:5 is a 100-nucleotide sequence representing the 5′ junction region of the sugar beet genomic DNA and the integrated transgene insert. SEQ ID NO:5 corresponds to nucleotide positions 951-1050 of SEQ ID NO:10.

SEQ ID NO:6 is a 100-nucleotide sequence representing the 3′ junction region of the integrated transgene insert and the sugar beet genomic DNA. SEQ ID NO:6 corresponds to nucleotide positions 12673-12772 of SEQ ID NO:10.

SEQ ID NO:7 is a 1050-nucleotide sequence representing the 5′ genomic flank region of the sugar beet genomic DNA and 50 nucleotides of the integrated transgene insert. SEQ ID NO:7 corresponds to nucleotide positions 1-1050 of SEQ ID NO:10.

SEQ ID NO:8 is a 1050-nucleotide sequence representing 50 nucleotides of the integrated transgene insert and the 3′ genomic flank region of the sugar beet genomic DNA. SEQ ID NO:8 corresponds to nucleotide positions 12673-13722 of SEQ ID NO:10.

SEQ ID NO:9 is a 11722-nucleotide sequence corresponding to the transgene insert of sugar beet event Bv_CSM63713. SEQ ID NO:9 corresponds to nucleotide positions 1001-12722 of SEQ ID NO:10.

SEQ ID NO:10 is a 13722-nucleotide sequence corresponding to the contig nucleotide sequence of the 5′ sugar beet genomic DNA sequence (SEQ ID NO:11), the transgene insert in event Bv_CSM63713 (SEQ ID NO:9), and the 3′ sugar beet genomic DNA sequence (SEQ ID NO:12).

SEQ ID NO:11 is a 1000-nucleotide sequence representing the 5′ flanking sugar beet genomic DNA up to the transgene insert (SEQ ID NO:9). SEQ ID NO:11 corresponds to nucleotide positions 1-1000 of SEQ ID NO:10.

SEQ ID NO:12 is a 1000-nucleotide sequence representing the 3′ flanking sugar beet genomic DNA after the transgene insert (SEQ ID NO:9). SEQ ID NO:12 corresponds to nucleotide positions 12723-13722 of SEQ ID NO:10.

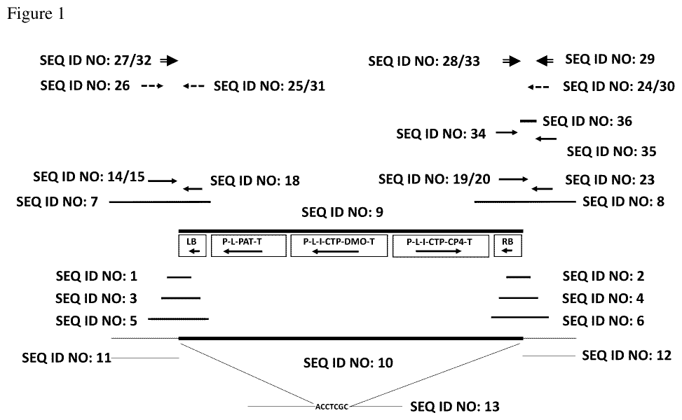

SEQ ID NO:13 is a 2007-nucleotide sequence representing wild type sugar beet genomic DNA at the location where the transgenic sequence was inserted in event Bv_CSM63713. Integration of the transgenic insert (SEQ ID NO:9) into the sugar beet genome resulted in a seven-nucleotide deletion (ACCTCGC) of genomic sequence in sugar beet event Bv_CSM63713, as shown in FIG. 1.

SEQ ID NO:14 is a 47-nucleotide sequence corresponding to a forward primer referred to as txht024d01-X used to identify sugar beet event Bv_CSM63713 DNA in a sample. The primer contains a 5′ 21-nucleotide oligo sequence used for Kompetitive Allele-Specific PCR (KASP). Nucleotides 20-47 of SEQ ID NO:14 correspond to positions 972-999 of SEQ ID NO:10 (Nucleotides 20-21 of the oligo tail (CT) also align with SEQ ID NO:10, together with nucleotides 22-47).

SEQ ID NO:15 is a 26-nucleotide sequence corresponding to the forward primer txht024d01-X as described in SEQ ID NO:14, but without the 21-nucleotide oligo sequence.

SEQ ID NO:16 is a 41-nucleotide sequence corresponding to a reverse primer referred to as sxcp4xxxs1-X used in Kompetitive Allele-Specific PCR (KASP) to detect the presence of the CP4 EPSPS gene in regenerated sugar beet events for event selection. Nucleotides 1-21 of SEQ ID NO: 16 are an oligonucleotide tail sequence.

SEQ ID NO:17 is 27-nucleotide sequence corresponding to a forward primer referred to as sxcp4xxxs1-C used in KASP to detect the presence of the CP4 EPSPS gene in regenerated sugar beet events for event selection.

SEQ ID NO 18 is a 26-nucleotide sequence corresponding to a reverse primer referred to as txht024d01-R used to identify sugar beet event Bv_CSM63713 DNA in a sample when in combination with SEQ ID NO: 14 or 15; it corresponds to positions 1004-1029 of SEQ ID NO:10 in a reverse orientation.

SEQ ID NO:19 is a 48-nucleotide sequence corresponding to a forward primer referred to as txht024d02-X used to identify sugar beet event Bv_CSM63713 DNA in a sample. The primer contains a 5′ 21-nucleotide oligo sequence used for Kompetitive Allele-Specific PCR (KASP). Nucleotides 22-48 of SEQ ID NO:19 correspond to positions 12697-12723 of SEQ ID NO:10.

SEQ ID NO:20 is a 27-nucleotide sequence corresponding to the forward primer txht024d02-X as described in SEQ ID NO:19, but without the 21-nucleotide oligo sequence.

SEQ ID NO:21 is a 43-nucleotide sequence corresponding to a reverse primer referred to as sxpatxxxs2-X used in Kompetitive Allele-Specific PCR (KASP) to detect the presence of the PAT gene in regenerated sugar beet events for event selection. Nucleotides 1-21 of SEQ ID NO: 21 are an oligonucleotide tail sequence.

SEQ ID NO:22 is 25-nucleotide sequence corresponding to a forward primer referred to as sxpatxxxs2-C used in KASP to detect the presence of the PAT gene in regenerated sugar beet events for event selection.

SEQ ID NO:23 is a 29-nucleotide sequence corresponding to a reverse primer referred to as txht024d02-R used to identify sugar beet event Bv_CSM63713 DNA in a sample when in combination with SEQ ID NO: 19 or 20; it corresponds to positions 12727-12755 of SEQ ID NO:10 in a reverse orientation.

SEQ ID NO:24 is a 44-nucleotide sequence corresponding to a reverse primer referred to as txht024s01-X used in a zygosity assay for sugar beet event Bv_CSM63713 DNA in a sample and hybridizes to a region of the sugar beet genome. The primer contains a 5′ 21-nucleotide oligo sequence used for Kompetitive Allele-Specific PCR (KASP). Nucleotides 22-37 of SEQ ID NO:24 correspond to positions 12723-12738 of SEQ ID NO:10 in a reverse orientation. Nucleotides 38-44 of SEQ ID NO:24 correspond to the seven-nucleotide wild-type sugar beet genomic DNA sequence (ACCTCGC) that was deleted in the sugar beet event Bv_CSM63713 as shown in SEQ ID NO:13 as depicted in FIG. 1.

SEQ ID NO:25 is a 45-nucleotide sequence corresponding to a reverse primer referred to as txht024s01-Y used in a zygosity assay for sugar beet event Bv_CSM63713 DNA in a sample. The primer contains a 5′ 21-nucleotide oligo sequence used for Kompetitive Allele-Specific PCR (KASP) Nucleotides 21-45 of SEQ ID NO:25 correspond to positions 1001-1025 of SEQ ID NO:10 in a reverse orientation (Nucleotide 21 of the oligo tail (T) also aligns with SEQ ID NO:10, together with nucleotides 22-45).

SEQ ID NO:26 is a 30-nucleotide sequence corresponding to a forward primer referred to as txht024s01-R used in a zygosity assay for sugar beet event Bv_CSM63713 DNA in a sample. It corresponds to positions 957-986 of SEQ ID NO:10.

SEQ ID NO:27 is a 42-nucleotide sequence corresponding to a forward primer referred to as txht024s02-X used in a zygosity assay for sugar beet event Bv_CSM63713 DNA in a sample and hybridizes to a region of the sugar beet genome. The primer contains a 5′ 21-nucleotide oligo tail sequence used for Kompetitive Allele-Specific PCR (KASP). Nucleotides 21-35 of SEQ ID NO:27 correspond to positions 986-1000 of SEQ ID NO:10. (Nucleotide 21 of the oligo tail, “T”, also aligns with SEQ ID NO:10, together with nucleotides 22-35). Nucleotides 36-42 of SEQ ID NO:27 correspond to the seven-nucleotide wild-type sugar beet genomic DNA sequence (ACCTCGC) that was deleted in the sugar beet event Bv_CSM63713 as described in SEQ ID NO:13.

SEQ ID NO:28 is a 48-nucleotide sequence corresponding to a forward primer referred to as txht024s02-Y used in a zygosity assay for sugar beet event Bv_CSM63713 DNA in a sample. Nucleotides 22-48 of SEQ ID NO:28 correspond to positions 12696-12722 of SEQ ID NO:10, and nucleotides 1-21 of SEQ ID NO: 28 are an oligonucleotide tail sequence.

SEQ ID NO:29 is a 30-nucleotide sequence corresponding to a reverse primer referred to as txht024s02-R used in a zygosity assay for sugar beet event Bv_CSM63713 DNA in a sample. It corresponds to positions 12743-12772 of SEQ ID NO:10 in a reverse orientation.

SEQ ID NO:30 is a 23-nucleotide sequence corresponding to SEQ ID NO:24, but without the 21-nucleotide oligo sequence. It is used in a zygosity assay for sugar beet event Bv_CSM63713 DNA in a sample and hybridizes to a region of the sugar beet genome.

SEQ ID NO:31 is a 24-nucleotide sequence corresponding SEQ ID NO:25, but without 21-nucleotide oligo sequence. It is used in a zygosity assay for sugar beet event Bv_CSM63713 DNA in a sample.

SEQ ID NO:32 is a 21-nucleotide sequence corresponding to SEQ ID NO:27, but without the 21-nucleotide oligo tail. It is used in a zygosity assay for sugar beet event Bv_CSM63713 DNA in a sample and hybridizes to a region of the sugar beet genome.

SEQ ID NO:33 is a 27-nucleotide sequence corresponding SEQ ID NO:28, but without the 21-nucleotide oligo sequence. It is used in a zygosity assay for sugar beet event Bv_CSM63713 DNA in a sample.

SEQ ID NO:34 is a 17-nucleotide sequence corresponding to a forward primer referred to as 2109_fwd1 used to identify sugar beet event Bv_CSM63713 DNA in a sample. It corresponds to positions 12686-12702 of SEQ ID NO:10.

SEQ ID NO:35 is a 24-nucleotide sequence corresponding to a reverse primer referred to as 2109_rev1 used to identify sugar beet event Bv_CSM63713 DNA in a sample; it corresponds to positions 12739-12762 of SEQ ID NO:10 in a reverse orientation.

SEQ ID NO:36 is a 16-nucleotide sequence corresponding to a probe referred to as 2109_probe1 and was used to identify sugar beet Bv_63713 event DNA in a sample; it corresponds to positions 12710-12725 of SEQ ID NO:10.

SEQ ID NO:37 is a 40-nucleotide sequence corresponding to a forward primer referred to as txdmoxxxxx-X used in Kompetitive Allele-Specific PCR (KASP) to detect the presence of the DMO gene in regenerated sugar beet events for event selection. Nucleotides 1-21 of SEQ ID NO: 40 are an oligonucleotide tail sequence.

SEQ ID NO:38 is 29-nucleotide sequence corresponding to a reverse primer referred to as txdmoxxxxx-C used in KASP to detect the presence of the DMO gene in regenerated sugar beet events for event selection.

SEQ ID NOs:39 and 40 are 39- and 25-nucleotide sequences corresponding to primers referred to as txmonlbxxx-X and txmonlbxxx-C, respectively, used in Kompetitive Allele-Specific PCR (KASP) to detect the presence of Agrobacterium T-DNA left border (LB) overread in regenerated sugar beet events for event selection. Nucleotides 1-21 of each primer are oligonucleotide tail sequences.

SEQ ID NOs:41 and 42 are 40- and 28-nucleotide sequences corresponding to primers referred to as txmonrbxxx-X and txmonrbxxx-C, respectively, used in Kompetitive Allele-Specific PCR (KASP) to detect the presence of Agrobacterium T-DNA right border (RB) overread in regenerated sugar beet events for event selection. Nucleotides 1-21 of each primer are oligonucleotide tail sequences.

SEQ ID NOs:43 and 44 are 42- and 23-nucleotide sequences corresponding to primers referred to as txvir2dd04-X and txvir2dd04-C, respectively, used in Kompetitive Allele-Specific PCR (KASP) to detect the presence of Agrobacterium in regenerated sugar beet events for event selection. Nucleotides 1-21 of each primer are oligonucleotide tail sequences.

SEQ ID NO:45 is a 24-nucleotide sequence corresponding to a forward primer referred to as GluA3-F for amplification of the glutamine synthetase, which serves as a sugar beet PCR internal control.

SEQ ID NO:46 is a 23-nucleotide sequence corresponding to a reverse primer referred to as GluA3-R for amplification of the glutamine synthetase, which serves as a sugar beet PCR internal control.

SEQ ID NO:47 is a 27-nucleotide sequence corresponding to a probe referred to GluD1 for amplification of the glutamine synthetase, which serves as a sugar beet PCR internal control.

SEQ ID NOs:48-77 are the nucleotide sequences for the genetic elements in the transgenic insert of sugar beet event Bv_CSM63713 and are further described in Table 15 hereinbelow.

### DETAILED DESCRIPTION

The following definitions, descriptions, and methods are provided to better define the invention and to guide those of ordinary skill in the art in the practice of the invention. Unless otherwise noted, terms are to be understood according to conventional usage by those of ordinary skill in the relevant art.

Herbicide tolerance is an important agronomic trait for effective weed control to maintain favorable crop growing conditions and crop yields, and is achieved by engineering of herbicide tolerance transgenes in crop plants using modern plant biotechnology techniques. Sugar beet event Bv_CSM61723 provides tolerance to three different herbicide chemistries through different modes of action for weed control and herbicide-resistant weed management.

Plant transformation techniques are used to insert foreign DNA (also known as transgenic DNA) randomly into a chromosome of the genome of a cell to produce a genetically engineered cell, also referred to as a “transgenic” or “recombinant” cell. Using this technique, many individual cells are transformed, each resulting in a unique transgenic event due to the random insertion of the foreign DNA into the genome. A transgenic plant is then regenerated from each individual transgenic cell. This results in every cell of the transgenic plant containing the uniquely inserted transgenic event as a stable part of its genome. This transgenic plant can then be used to produce progeny plants, each containing the unique transgenic event.

Sugar beet event Bv_CSM63713 is provided. The event Bv_CSM63713 was produced by Agrobacterium-mediated transformation of sugar beet shoot meristematic tissue by: (i) transformation of thousands of sugar beet cells with four different DNA constructs that included three expression cassettes (each expression cassette having been selected after individual testing followed by testing in combination with the other two expression cassettes), (ii) regeneration of a population of transgenic plants each containing a unique transgenic event, and (iii) rigorous multi-year event selection involving the testing and analysis of the molecular characteristics, herbicide tolerance efficacy, and agronomic properties in a variety of genetic backgrounds for hundreds of events through tens of thousands of plants. Sugar beet event Bv_CSM63713 was thus produced and selected as a uniquely superior event useful for broad-scale agronomic commercial purposes.

As used herein, an “expression cassette” or “cassette” or “transgene cassette” is a recombinant DNA molecule or sequence comprising a combination of distinct elements for expressing an RNA and/or protein encoded by the coding sequence of a transgene in a transformed plant cell or transformed plant comprising the transgene. As provided herein, an “expression cassette” or “cassette” or “transgene cassette” includes one or more regulatory element(s) operably linked to a coding or transcribable DNA sequence. The regulatory elements can include a promoter, a leader, 5′ untranslated region (5′ UTR), intron and/or a 3′ untranslated region (3′ UTR) region. The “expression cassette” or “cassette” or “transgene cassette” is recombinant and heterologous with respect to the transformed plant cell genome. For purposes of the present disclosure, such an “expression cassette” or “cassette” or “transgene cassette” is a recombinant DNA molecule or sequence that encodes a protein for conferring tolerance to at least one class of herbicides as described herein. Table 15 provides a list of the genetic elements contained in the three transgene cassettes in the transgenic insert (SEQ ID NO:9) of sugar beet event Bv_CSM63713.

The act of inserting the transgenic DNA into the genome of the sugar beet plant is accomplished by plant transformation methods known in the art and creates a new transgenic genomic DNA sequence, known as a “transgenic event” or an “event”. The DNA sequence of the event comprises the inserted foreign DNA (referred to as the “transgenic insert”) and the genomic DNA adjacent to, or “flanking,” the transgenic insert on either side of the insertion location (referred to as the “flanking DNA”). As used herein, the term “flanking” in reference to a transgenic event refers to the plant genomic sequence(s) adjacent to the transgenic DNA insertion in the genome of a transformed plant, plant part, plant tissue, or plant cell comprising the transgenic event on the 5′ and/or 3′ end(s) of the transgenic event insertion. Likewise, “flanking DNA” refers to a length of genomic DNA sequence adjacent to the transgenic DNA insertion in the genome of the transformed event on the 5′ and/or 3′ end(s) of the insertion. A “5′ flank”, therefore, means the sugar beet genomic DNA sequence adjacent to and upstream (or on the 5′ end) of the transgenic DNA insertion. For example, a “5′ flank” can include the sugar beet genomic DNA sequence immediately adjacent to and upstream (on the 5′ end) of the transgenic insertion, or any sugar beet genomic DNA sequence upstream (on the 5′ end) of the transgenic insertion that is not immediately adjacent to the transgenic insertion but is within about 5000 nucleotides, within about 3000 nucleotides, or within about 1000 nucleotides upstream of the transgenic insertion. Likewise, a “3′ flank” means the sugar beet genomic DNA sequence adjacent to and downstream (or on the 3′ end) of the transgenic insert. For example, a “3′ flank” can include the sugar beet genomic DNA sequence immediately adjacent to and downstream (on the 3′ end) of the transgenic insertion, or any sugar beet genomic DNA sequence downstream (on the 3′ end) of the transgenic insertion that is not immediately adjacent to the transgenic insertion but is within about 5000 nucleotides, within about 3000 nucleotides, or within about 1000 nucleotides downstream of the transgenic insertion. The DNA sequence of an event is unique to and specific for the event and can be readily identified when compared to other DNA sequences, such as that of other events or untransformed sugar beet genomic DNA. Sugar beet event Bv_CSM63713 has the new and unique DNA sequence provided as SEQ ID NO:10, which comprises a contiguous sequence comprising the 5′ sugar beet genomic flanking sequence provided as SEQ ID NO:11, the transgenic insert sequence provided as SEQ ID NO:9, and the 3′ sugar beet genomic flanking DNA sequences provided in SEQ ID NO:12. Sugar beet event Bv_CSM63713 is thus a DNA molecule that is an integral part of the chromosome of transgenic sugar beet cells and plants comprising the event and as such is static and may be passed on to progeny cells and plants.

Progeny of the original transformed cell and plant that comprise sugar beet event Bv_CSM63713 are also provided. Such progeny may be produced by cell tissue culture, by selfing of a sugar beet plant comprising the sugar beet event Bv_CSM63713, or by sexual outcrossing between a sugar beet plant comprising sugar beet event Bv_CSM63713 and another plant that does or does not contain the event, or by any other method known in the art including any plant cell or tissue culture method, wherein the progeny includes sugar beet event Bv_CSM63713. Such other plant may be a transgenic plant comprising the same or different event(s) or a nontransgenic plant, such as one from a different variety. Sugar beet event Bv_CSM63713 is passed from the original parent through each generation to the progeny. A “transgenic plant” or “plant”, therefore, can be the original transformant plant regenerated from the transformed plant cell and comprising the transgenic DNA and event, or a progeny plant of the original transformant plant, which may be separated from the transformant by one or more generations, that retains the transgenic DNA and event at the same specific location and sequence context in the plant's genome.

As used herein, the term “beet” refers to Beta vulgaris, including all Beta vulgaris subspecies and, in addition, includes all plant species that can be bred with Beta vulgaris. It includes Beta vulgaris subsp. adanensis, Beta vulgaris subsp. maritina, and Beta vulgaris subsp. vulgaris, Beta macrocarpa, and Beta patula. Within the Beta vulgaris subsp. vulgaris, it includes: 1) the Altissima group, such as sugar beet; 2) the Cicla group, such as spinach beet or chard; 3) the Flavescens group, such as swiss chard; 4) the Conditiva group, such as beetroot or garden beet; and 5) the Crassa or the fodder beet group, such as mangelwurzel.

The present disclosure describes introduction of event Bv_CSM63713 into sugar beet, and thus the term “sugar beet event Bv_CSM63713” is used to refer to the event herein. However, those of skill in the art will understand that event Bv_CSM63713 could readily be introduced into other Beta vulgaris and plant species that can be bred with Beta vulgaris by sexually crossing or outcrossing between a sugar beet plant containing sugar beet event Bv_CSM63713 and another plant that does not contain sugar beet event Bv_CSM63713, or by any other method known in the art. For example, to introduce event Bv_CSM63713 into fodder beet, one would cross-pollinate a plant containing or comprising event Bv_CSM63713 with a fodder beet plant, which may be accomplished or facilitated by human intervention, for example, by human hands collecting the pollen from a plant that contains event Bv_CSM63713 and placing the pollen onto the style or stigma of a fodder beet plant that does not contain event Bv_CSM63713, wherein the event Bv_CSM63713 is passed from the male parent comprising Bv_CSM63713 to the fodder beet progeny; or by human hands and/or human actions removing, destroying, or covering the stamen or anthers of a fodder beet plant (e.g., by manual intervention or by application of a chemical gametocide) so that natural self-pollination is prevented and cross-pollination with pollen from a plant containing event Bv_CSM63713 would take place in order for fertilization to occur; or by human placement of pollinating insects in a position for directed pollination (e.g., by placing beehives in orchards or fields or by caging plants with pollinating insects); or by human opening or removing of parts of the fodder beet flower to allow for placement or contact of foreign pollen from a plant containing or comprising event Bv_CSM63713 on the style or stigma of the fodder beet plant; by selective placement of plants (e.g., intentionally planting plants in pollinating proximity); and/or by application of chemicals to precipitate flowering or to foster receptivity (of the stigma for pollen). Besides directed cross-pollination one could allow flowering of a sugar beet containing sugar beet event Bv_CSM63713 and a fodder beet at synchronized time points and in close proximity to achieve random cross-pollination and subsequent selection of a progeny containing sugar beet event Bv_CSM63713.

Sugar beet event Bv_CSM63713 provides to sugar beet cells, plants, and seeds that comprise the event tolerance to benzoic acid auxin herbicides such as dicamba; 5-enolpyruvylshikimate-3-phosphate synthase (EPSPS) inhibitors such as glyphosate; and inhibitors of glutamine synthetase such as glufosinate. Sugar beet event Bv_CSM63713 contains three expression cassettes. Table 15 in Example 7 provides a list of the elements contained in SEQ ID NO:10, as illustrated in FIG. 1.

As used herein, the term “derived” or “derived from” in reference to a particular DNA molecule, amplicon or sequence in relation to a sugar beet plant, plant part, seed, progeny, cell and/or sugar beet plant product, such as a commodity product, means that the DNA molecule, amplicon or sequence is taken, purified, isolated, or made, directly or indirectly, from such sugar beet plant, plant part, seed, progeny, cell and/or sugar beet plant product, such as a commodity product. Alternatively, the term “derived” or “derived from” in reference to a sugar beet plant product, such as a commodity product, in relation to sugar beet plant, plant part, seed, progeny, or cell, means that the sugar beet plant product is taken, purified, isolated, or made, directly or indirectly, from such sugar beet plant, plant part, seed, progeny, or cell.

“Capable of being detected” refers to the ability of a particular DNA molecule, segment or sequence to be detected in a sample, such as by amplification and determining its presence, size or sequence such as by DNA sequence analysis, and/or binding of a probe to the target DNA molecule, segment or sequence.

A “sample” is intended to refer to any composition comprising or derived from, either directly or indirectly, a biological sample, source, or material. The sample may generally comprise sugar beet DNA and/or substantially or completely pure, purified, or isolated sugar beet DNA. A “biological sample” contains biological materials, including but not limited to DNA obtained or derived from, either directly or indirectly, the genome of a sugar beet cell(s), tissue(s), seed(s), plant(s), plant part(s) and/or sugar beet plant product(s), such as a commodity product(s). Such sugar beet cell(s), tissue(s), seed(s), plant(s), plant part(s) and/or sugar beet plant product(s), such as a commodity product(s), may comprise sugar beet event Bv_CSM63713, or DNA molecule(s) and/or DNA segment(s) comprising sugar beet event Bv_CSM63713. In some embodiments, a sample or biological sample may comprise sugar beet cell(s), sugar beet tissue(s), sugar beet seed(s), sugar beet plant(s), sugar beet plant part(s) and/or sugar beet plant product(s), whose cells or cellular membranes have been fractured (e.g., disrupted or opened) to release the contents of the sugar beet cell(s) including genomic DNA or proteins and/or make the contents of the sugar beet cell(s) including genomic DNA or proteins accessible or usable for assays or testing. “Directly” refers to directly obtaining DNA by a skilled artisan from the sugar beet genome by fracturing sugar beet cells (or by obtaining samples of sugar beet that contain fractured sugar beet cells) and exposing or using the genomic DNA or protein from sugar beet cells for the purposes of detection. “Indirectly” refers to obtaining by a skilled artisan a target or specific reference DNA (e.g., a novel and unique junction segment(s) described herein as being diagnostic for the presence of the event Bv_CSM63713) in a particular sample, by means other than by obtaining directly via fracturing of sugar beet cells or obtaining a sample of sugar beet that contains fractured sugar beet cells. Such indirect means include, but are not limited to, amplification of a DNA segment that contains a DNA sequence targeted by a particular probe(s) and/or primer set(s) designed to bind with specificity to or near the target sequence, or amplification of a DNA segment comprising all or part of a target sequence that can be measured and characterized (e.g., measured by migration or separation from other segments of DNA and/or identification in an effective matrix, such as an agarose or acrylamide gel or the like, or characterized by direct sequence analysis of the amplicon(s), or cloning of the amplicon(s) into a vector(s) and direct sequencing of the inserted amplicon(s) present within such vector(s)).

As used herein, the term “recombinant” refers to a non-natural DNA, protein, or organism that would not normally be found in nature and was created by human intervention. As used herein, a “recombinant DNA molecule” is a DNA molecule comprising a combination of DNA molecules that would not naturally occur together and is the result of human intervention, for example, a DNA molecule that is comprised of a combination of at least two DNA molecules heterologous to each other, such as a DNA molecule that comprises a transgene and the plant genomic DNA adjacent to the transgene. An example of a recombinant DNA molecule is a DNA molecule comprising at least one sequence selected from SEQ ID NOs.1-10. As used herein, a “recombinant plant” is a plant that would not normally exist in nature, is the result of human intervention, and contains a transgenic DNA molecule. As a result of such genomic alteration, the recombinant plant is something new and distinctly different from the related wild-type plant. An example of a recombinant plant is a sugar beet plant containing the sugar beet event Bv_CSM63713.

As used herein, the term “transgene” refers to a DNA molecule artificially incorporated into an organism's genome as a result of human intervention, such as by plant transformation methods. A transgene may be heterologous to the organism. The term “transgenic insert” as used herein refers to the foreign DNA inserted by plant transformation techniques into the sugar beet genome to produce sugar beet event Bv_CSM63713. The sequence for the transgenic insert of sugar beet event Bv_CSM63713 is provided as SEQ ID NO:9. The term “transgenic” refers to comprising a transgene, for example a “transgenic plant” refers to a plant comprising a transgene.

As used herein, the term “heterologous” refers to a first molecule not normally associated with a second molecule or an organism in nature. For example, a DNA molecule may be from a first species and inserted into the genome of a second species. The DNA molecule would thus be heterologous to the genome and the organism.

As used herein, the term “chimeric” refers to a single DNA molecule produced by fusing a first DNA molecule to a second DNA molecule, where neither first nor second DNA molecule would normally be found in that configuration fused to the other. The chimeric DNA molecule is thus a new DNA molecule not normally found in nature. An example of a chimeric DNA molecule is a DNA molecule comprising at least one sequence selected from SEQ ID NOs:1-10.

As used herein, the term “isolated” refers to separating a molecule from other molecules that are normally associated with it in its native or natural state. The term “isolated” thus may refer to a DNA molecule that has been separated from other DNA molecule(s) that it is associated with in its native or natural state. Such a DNA molecule may be present in a recombined state, such as a recombinant DNA molecule. Thus, a DNA molecule removed from its natural state and fused to another DNA molecule with which it is not normally associated would be an isolated DNA molecule. Such an isolated DNA molecule could result from the use of biotechnology techniques, such as making recombinant DNA or integrating a foreign DNA molecule into the chromosome of a cell, plant, or seed.

DNA molecules, fragments, and their corresponding DNA sequences are provided. As used herein, the terms “DNA” and “DNA molecule” refer to a deoxyribonucleic acid (DNA) molecule. A DNA molecule may be of genomic or synthetic origin and is by convention from the 5′ (upstream) end to the 3′ (downstream) end. As used herein, the term “DNA sequence” refers to the nucleotide sequence of a DNA molecule, i.e. the sequence of consecutive nucleotides in the DNA molecule. As used herein in reference to nucleotides of a polynucleotide or DNA sequence or molecule, the terms “consecutive” and “contiguous” are interchangeable and synonymous and refer to the 5′ to 3′ order of nucleotides in a polynucleotide or DNA sequence, strand or molecule without any gap or interruption between them. The nomenclature used is that required by Title 37 of the United States Code of Federal Regulations § 1.822 and set forth in the tables in WIPO Standard ST.25 (1998), Appendix 2, Tables 1 and 3. By convention, DNA sequences and fragments thereof are disclosed with reference to only one strand of the two complementary DNA sequence strands. By implication and intent, the complementary sequences of the sequences provided here (the sequences of the complementary strand), also referred to in the art as the reverse complementary sequences, are within the scope of the invention and are expressly intended to be within the scope of the subject matter claimed. Thus, as used herein references to SEQ ID NOs:1-10 and fragments thereof include and refer to the sequence of the complementary strand and fragments thereof. FIG. 1 is an illustration of the transgenic DNA inset in the genome of a sugar beet plant comprising event Bv_CSM63713, and the relative positions of SEQ ID NOs:1-10 arranged 5′ to 3′.

Also provided is a nucleic acid molecule comprising a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% identical to the full length of SEQ ID NOs:1-12.

For example, a nucleic acid molecule is provided comprising a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% identical to the full length of SEQ ID NO:10 or to the full length of SEQ ID NO: 9.

As used herein, the term “fragment” refers to a smaller piece or sequence of a larger or whole DNA molecule or sequence. For example, a fragment of any one of SEQ ID NOs:1-12 may include a sequence that are at least about 10 consecutive nucleotides, at least about 11 consecutive nucleotides, at least about 12 consecutive nucleotides, at least about 13 consecutive nucleotides, at least about 14 consecutive nucleotides, at least about 15 consecutive nucleotides, at least about 16 consecutive nucleotides, at least about 17 consecutive nucleotides, at least about 18 consecutive nucleotides, at least about 19 consecutive nucleotides, at least about 20 consecutive nucleotides, at least about 21 consecutive nucleotides, at least about 22 consecutive nucleotides, at least about 23 consecutive nucleotides, at least about 24 consecutive nucleotides, at least about 25 consecutive nucleotides, at least about 30 consecutive nucleotides, at least about 35 consecutive nucleotides, at least about 40 consecutive nucleotides, at least about 45 consecutive nucleotides, at least about 50 consecutive nucleotides, at least about 60 consecutive nucleotides, at least about 70 consecutive nucleotides, at least about 80 consecutive nucleotides, at least about 90 consecutive nucleotides, at least about 100 consecutive nucleotides, at least about 200 consecutive nucleotides, at least about 300 consecutive nucleotides, at least about 400 consecutive nucleotides, or at least about 500 consecutive nucleotides of the larger, whole or complete DNA molecule or sequence.

For example, a “fragment” of the transgenic insert sequence (SEQ ID NO: 9) of sugar beet event Bv_CSM63713 can comprise at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 60, at least about 70, at least about 80, at least about 90, at least about 100, at least about 200, at least about 300, at least about 400, or at least about 500 consecutive nucleotides of SEQ ID NO: 9. In addition, the present disclosure encompasses nucleotide sequences that are at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% identical to SEQ ID NO: 9 or any fragment thereof.

Similarly, a fragment of the 5′ flank (SEQ ID NO: 11) or 3′ flank (SEQ ID NO: 12) of sugar beet event Bv_CSM63713 can comprise at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 60, at least about 70, at least about 80, at least about 90, at least about 100, at least about 200, at least about 300, at least about 400, or at least about 500 consecutive nucleotides of SEQ ID NO: 11 or 12. In addition, the present disclosure encompasses nucleotide sequences that are at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% identical to SEQ ID NO: 11 or 12 or any fragment of either thereof.

The DNA sequence for the transgenic insert of sugar beet event Bv_CSM63713 is provided as SEQ ID NO:9. The DNA sequence of the transgenic insert and the sugar beet genomic DNA flanking each side of the transgenic insert is provided as SEQ ID NO:10. The DNA sequences of a portion of flanking DNA and the 5′ end of the transgenic insert are provided as SEQ ID NO:1, SEQ ID NO:3, SEQ ID NO:5, and SEQ ID NO:7. The DNA sequences of a portion of flanking DNA and the 3′ end of the transgenic insert are provided as SEQ ID NO:2, SEQ ID NO:4, SEQ ID NO:6, and SEQ ID NO:8.

Sugar beet event Bv_CSM63713 is characterized as a transgenic insertion into a single locus in the sugar beet genome, resulting in two new junctions (or joining or connection points). The DNA sequence of the region spanning the connection by phosphodiester bond linkage of one end of the transgenic insert to the flanking sugar beet genomic DNA is referred to herein as a “junction.” A junction is the connection point or covalent linkage of one end of the transgenic insert and the flanking genomic DNA as one contiguous molecule, and is formed by the insertion of a heterologous nucleic acid molecule into the sugar beet genomic DNA. One junction is found at the 5′ end of the transgenic insert and the other is found at the 3′ end of the transgenic insert, referred to herein as the 5′ and 3′ junctions, respectively. A “junction sequence” refers to a DNA sequence of any length of consecutive nucleotides that spans the 5′ or 3′ junction of an event in a plant genome. For a “junction sequence” to be specific to a junction between a transgenic event and a flanking genomic sequence, the junction sequence will generally comprise a sufficient number of consecutive nucleotides at one end of the insertion and a sufficient number of consecutive nucleotides of the flanking genomic sequence. According to some embodiments, a “junction sequence” may comprise (i) at least five (5) consecutive nucleotides, at least ten (10) consecutive nucleotides, at least fifteen (15) consecutive nucleotides, at least twenty (20) consecutive nucleotides, at least twenty five (25) consecutive nucleotides, at least thirty (30) consecutive nucleotides, at least thirty five (35) consecutive nucleotides, at least forty (40) consecutive nucleotides, at least forth five (45) consecutive nucleotides, or at least fifty (50) consecutive nucleotides at one end of the insertion and (ii) at least five (5) consecutive nucleotides, at least ten (10) consecutive nucleotides, at least fifteen (15) consecutive nucleotides, at least twenty (20) consecutive nucleotides, at least twenty five (25) consecutive nucleotides, at least thirty (30) consecutive nucleotides, at least thirty five (35) consecutive nucleotides, at least forty (40) consecutive nucleotides, at least forth five (45) consecutive nucleotides, or at least fifty (50) consecutive nucleotides of the flanking genomic DNA sequence, although it is understood that any length of consecutive nucleotides spanning a junction of a transgenic event in a plant genome may be a junction sequence. Junction sequences of sugar beet event Bv_CSM63713 are apparent to, and a variety of junction sequences of sugar beet event Bv_CSM63713 can be determined by, one of skill in the art using SEQ ID NO:10. In SEQ ID NO:10, the 5′ junction is at nucleotides 1000-1001, and the 3′ junction is at nucleotides 12,722-12,723. Examples of junction sequences of sugar beet event Bv_CSM63713 are provided as SEQ ID NOs:1-8. FIG. 1 illustrates the physical arrangement of SEQ ID NOs:1-10 arranged from 5′ to 3′. The junction sequences of sugar beet event Bv_CSM63713 may be present as part of the genome of a plant, seed, plant part, progeny or plant cell containing sugar beet event Bv_CSM63713. The identification of any one or more of SEQ ID NOs:1-10 in a sample from a plant, plant part, seed, progeny or cell indicates that the DNA was obtained from sugar beet containing sugar beet event Bv_CSM63713 and is diagnostic for the presence of sugar beet event Bv_CSM63713.

The junction sequences described herein are diagnostic for the presence of all or part of sugar beet event Bv_CSM63713. Thus, the identification or detection, directly or indirectly, of one or more of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, and SEQ ID NO: 10 in a sample or DNA molecule derived from a sugar beet plant, plant part, seed, progeny, cell, or a commodity product is diagnostic that the sugar beet plant, plant part, seed, progeny, cell, or a commodity product has or comprises all or part of sugar beet event Bv_CSM63713. The identification or detection, directly or indirectly, of a 5′ junction sequence and/or a 3′ junction sequence (each as provided or described herein) in a sample or DNA molecule derived from a sugar beet plant, plant part, seed, progeny, cell, or a commodity product is diagnostic that the sugar beet plant, plant part, seed, progeny, cell, or a commodity product has or comprises sugar beet event Bv_CSM63713. The present disclosure thus provides a DNA molecule that comprises at least one of the nucleotide sequences provided as SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10. Any segment of DNA derived from transgenic sugar beet event Bv_CSM63713 that is sufficient to include at least one of the sequences provided as SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10 is within the scope of the present disclosure. In addition, any DNA or polynucleotide molecule or sequence comprising a sequence complementary to any of the sequences described herein is also within the scope of the present disclosure.

The plants, seeds, plant cells, plant parts, progeny and commodity products may be used for detection of DNA or protein molecules indicative of the presence of sugar beet event Bv_CSM63713. Provided are illustrative DNA molecules that can be used either as primers or probes for detecting the presence of sugar beet event Bv_CSM63713 in a sample. Such primers or probes are specific for a target nucleic acid sequence and as such are useful for the identification of sugar beet event Bv_CSM63713 by the methods described herein. A primer or probe can hybridize to a target polynucleotide sequence to allow for specific detection or amplification of a polynucleotide molecule that comprises, or is covalently linked and associated with, the target polynucleotide sequence. The target polynucleotide sequence may comprise all or part of sugar beet event Bv_CSM63713, a junction sequence, and/or flanking genomic DNA. Probes and primers according to the present disclosure may have (i) complete or 100% sequence complementarity (i.e., 100% complementary) to a target polynucleotide sequence or (ii) incomplete sequence complementarity to a target polynucleotide, such as at least 60% complementary, at least 65% complementary, at least 70% complementary, at least 75% complementary, at least 80% complementary, at least 85% complementary, at least 90% complementary, at least 95% complementary, or at least 99% complementary to the target polynucleotide sequence as long as the probe or primer has sufficient complementarity to the target polynucleotide sequence to hybridize to the target polynucleotide sequence under stringent hybridization conditions that are suitable and necessary for use of the probe or primer in the relevant amplification or detection assay, reaction or method. As understood in the art, the percentage complementarity of a primer or probe may be lower if the length of the primer or probe is longer, and depends on the stringency and use. Provided are illustrative polynucleotide molecules that can be used either as primers or probes for detecting the presence of sugar beet event Bv_CSM63713 in a sample. Detection of the presence of sugar beet event Bv_CSM63713 may be done by using methods known in the art, such as thermal or isothermal amplification of nucleic acid or nucleic acid hybridization techniques (such as Southern analysis).

A “primer” is a DNA molecule that is designed for use in annealing or hybridization methods that involve an amplification reaction. An amplification reaction is an in vitro reaction that amplifies template DNA to produce an amplicon. As used herein, an “amplification product” or “amplified DNA” or “amplicon” is a DNA molecule that has been synthesized using amplification techniques as further described herein, which is directed to a target nucleic acid or DNA molecule that is part of a template nucleic acid molecule. Amplification or amplifying refers to making multiple copies of a target DNA molecule or segment from a template DNA. For example, to determine whether a sugar beet plant, plant part, seed, progeny or plant cell, resulting from selfing or outcross of a parent comprising sugar beet event Bv_CSM63713 contains sugar beet event Bv_CSM63713, DNA may be extracted from the sugar beet plant tissue sample and subjected to an amplification reaction or method using a pair of primers that are specific for a target sequence that is uniquely associated or part of sugar beet event Bv_CSM63713, such as, for example, a first primer derived from a genomic DNA sequence in the region flanking the heterologous inserted DNA of sugar beet event Bv_CSM63713 that is elongated by polymerase 5′ to 3′ in the direction of the inserted DNA, and a second primer derived from the heterologous inserted DNA molecule that is elongated by the polymerase 5′ to 3′ in the direction of the flanking genomic DNA from which the first primer is derived. The amplicon may range in length depending on the length of the intervening polynucleotide or DNA sequence between the two primer target sequences in the template DNA molecule. Alternatively, a primer pair can be derived from the genomic sequence on both sides of the inserted heterologous DNA so as to produce an amplicon that includes the entire insert polynucleotide sequence (e.g., a forward primer targeted to the genomic portion on the 5′ end of SEQ ID NO:10 (i.e. upstream of SEQ ID NO:9) and a reverse primer targeted to the genomic portion on the 3′ end of SEQ ID NO:10 (i.e. downstream of SEQ ID NO:9) that amplifies a DNA molecule comprising the inserted DNA sequence (SEQ ID NO:9) identified herein in the sugar beet event Bv_CSM63713 genome. The use of the term “amplicon” specifically excludes primer dimers that may be formed in a DNA amplification reaction.

The amplicon described herein may comprise a DNA sequence comprising one or more of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, or a fragment of any of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10 wherein the fragment is at least 10 nucleotides in length and comprises nucleotides 1,000-1,001 or 12,722-12,723 of SEQ ID NO:10. According to present embodiments, the sequence of an amplicon comprises at least one junction sequence or two junction sequences, such as a 5′ junction sequence and/or a 3′ junction sequences for sugar beet event Bv_CSM63713. Amplification and detection of such an amplicon is indictive or diagnostic for sugar beet event Bv_CSM63713.

A primer is typically designed to hybridize to a complementary target DNA strand to form a hybrid between the primer and the target DNA strand. The presence of a primer is a point of recognition by a polymerase to begin extension of the primer (i.e., polymerization of additional nucleotides into a lengthening nucleotide molecule) using as a template the target DNA strand. Primer pairs refer to use of two primers binding opposite strands of a double stranded nucleotide segment for the purpose of amplifying the polynucleotide segment between the positions targeted for binding by the individual members of the primer pair, typically in a thermal amplification reaction or other conventional nucleic-acid amplification methods.

To detect the presence or absence of sugar beet event Bv_CSM63713, the target positions and/or the intervening region or sequence of a template DNA molecule may comprise at least one junction sequence and/or at least a portion of the insert of sugar beet event Bv_CSM63713. To detect the absence of sugar beet event Bv_CSM63713, the target positions and/or the intervening region or sequence of a template DNA molecule may comprise sugar beet genomic DNA that does not include a junction sequence or any portion of the insert of sugar beet event Bv_CSM63713. Thus, the presence or absence of an amplicon with a primer pair may be diagnostic of the presence or absence, respectively, of sugar beet event Bv_CSM63713 in a DNA molecule or sample, or vice versa. This may also be possible with more than one primer pair. For example, a first primer pair may produce a first amplicon if sugar beet event Bv_CSM63713 is present, and a second primer pair may produce a second amplicon if sugar beet event Bv_CSM63713 is absent or not present. Alternatively, the size of an amplicon produced in an amplification reaction may also be diagnostic of the presence or absence of sugar beet event Bv_CSM63713 in a DNA molecule or sample—e.g., a primer pair may produce a first amplicon of a first size if sugar beet event Bv_CSM63713 is present or a second amplicon of a second size if sugar beet event Bv_CSM63713 is absent and not present; or a first primer pair may produce a first amplicon of a first size if sugar beet event Bv_CSM63713 is present, and a second primer pair may produce a second amplicon of a second size if sugar beet event Bv_CSM63713 is absent or not present. According to some of these embodiments, at least two primer pairs may be used wherein at least one of the primer pairs is used as an internal control and is not associated with sugar beet event Bv_CSM63713.

According to present embodiments, a primer pair to detect the presence or absence of all or part of sugar beet event Bv_CSM63713 in a DNA molecule or sample comprises a first primer and a second primer, wherein the first primer is complementary to a 5′ flanking genomic DNA sequence and the second primer is complementary to a sequence within the transgenic insert; or wherein the first primer is complementary to a 5′ flanking genomic DNA sequence and the second primer is complementary to a 3′ flanking genomic DNA sequence; or wherein the first primer is complementary to a sequence within the transgenic insert and the second primer is complementary to a 3′ flanking genomic DNA sequence. Each reference in this paragraph to a primer complementary to a 5′ flanking genomic DNA sequence, a 3′ flanking genomic DNA sequence, or a sequence within the transgenic insert of sugar beet event Bv_CSM63713 is also intended to potentially include a primer complementary to the reverse complement or opposing strand of the respective 5′ flanking genomic DNA sequence, 3′ flanking genomic DNA sequence, or sequence within the transgenic insert of sugar beet event Bv_CSM63713.

Illustrative DNA molecules useful as primers are provided as SEQ ID NO:19/20 and SEQ ID NO:23; SEQ ID NO:14/15 and SEQ ID NO:18, SEQ ID NO:34 and SEQ ID NO:35, SEQ ID NO:26 and SEQ ID NO:25/31, and SEQ ID NO:28/33 and SEQ ID NO:29. For example, illustrative event-specific primers for PCR to identify event Bv_CSM63713 are provided as SEQ ID NO:14 and SEQ ID NO:18, SEQ ID NO:19 and SEQ ID NO:23, and SEQ ID NO:34 and SEQ ID NO:35. Illustrative primers that may be used for analysis of the 5′ (left) junction region are provided as SEQ ID NO:14 and SEQ ID NO:18; and illustrative primers that may be used for analysis of the 3′ (right) junction region are provided as SEQ ID NO:19 and SEQ ID NO:23, and SEQ ID NO:34 and SEQ ID NO:35. Illustrative primers that may be used for zygosity testing for event Bv_CSM63713 are provided as SEQ ID NO:26, SEQ ID NO:25 and SEQ ID NO:24; and SEQ ID NO:27, SEQ ID NO:28 and SEQ ID NO:29.

The primer pairs provided as SEQ ID NO:19/20 and SEQ ID NO:23; SEQ ID NO:14/15 and SEQ ID NO:18, and SEQ ID NO:34 and SEQ ID NO:35 are useful as a first DNA molecule and a second DNA molecule, where the first DNA molecule is a fragment of the transgenic insert DNA sequence of SEQ ID NO:10 and the second DNA molecule is a fragment of the flanking DNA sequence of SEQ ID NO:10, and each are of sufficient length to function as DNA primers when used together in an amplification reaction with DNA containing sugar beet event Bv_CSM63713 to produce an amplicon diagnostic for sugar beet event Bv_CSM63713 in a sample. An amplicon diagnostic for event Bv_CSM63713 comprises a sequence not naturally found in the sugar beet genome. Primer pairs may also be defined as comprising a first and second DNA molecule, wherein the first DNA molecule is a fragment of the sugar beet genomic portion of SEQ ID NO:10 and the second DNA molecule is a fragment of the transgene portion of SEQ ID NO:10 (or SEQ ID NO:9), and each are of sufficient length to function as DNA primers when used together in an amplification reaction with DNA containing sugar beet event Bv_CSM63713 to produce an amplicon diagnostic for sugar beet event Bv_CSM63713 in a sample. A primer may further comprise an oligo tail sequence such as those used in the Kompetitive Allele-Specific PCR (KASP™) method. The allele-specific primers each harbor a unique tail sequence that corresponds with a universal FRET (fluorescence resonant energy transfer) cassette; one labelled with FAM™ dye and the other with HEX™ dye. During thermal cycling, the relevant allele-specific primer binds to the template and elongates, thus attaching the tail sequence to the newly synthesized strand. The complement of the allele-specific tail sequence is then generated during subsequent rounds of PCR, enabling the FRET cassette to bind to the DNA. The FRET cassette is no longer quenched and emits fluorescence. Examples of primers comprising an oligo tail sequence are SEQ ID NO:14, SEQ ID NO:19, SEQ ID NO:24, SEQ ID NO:25 and SEQ ID NO:27. Examples of primers corresponding to SEQ ID NO:14, SEQ ID NO:19, SEQ ID NO:24, SEQ ID NO:25 and SEQ ID NO:27, but that do not contain an oligo tail sequence are SEQ ID NO:15, SEQ ID NO:20, SEQ ID NO:30, SEQ ID NO:31 and SEQ ID NO:32.

A “probe” is a nucleic acid molecule that is complementary to a strand of a target nucleic acid and useful in hybridization detection methods. Probes include not only deoxyribonucleic or ribonucleic acids but also polyamides and other probe materials that bind specifically to a target DNA sequence and the detection of such binding can be useful in detecting the presence or absence of the target DNA sequence. A probe may be attached to a conventional detectable label or reporter molecule, such as a radioactive isotope, ligand, chemiluminescent agent, or enzyme.

An illustrative DNA sequence useful as a probe for detecting sugar beet event Bv_CSM63713 is provided as SEQ ID NO:36. In some embodiments, a DNA molecule that functions as a probe comprises a nucleotide sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, a complement of any of the foregoing or a fragment of any of the foregoing. In other embodiments, a DNA molecule comprises a polynucleotide segment of sufficient length to function as a DNA probe specific for at least one of: a) a 5′ junction sequence between flanking sugar beet genomic DNA and the transgenic insert of sugar beet event Bv_CSM63713; b) a 3′ junction sequence between the transgenic insert of sugar beet event Bv_CSM63713 and flanking sugar beet genomic DNA; c) SEQ ID NO:9; or d) a fragment of SEQ ID NO:9 comprising a sufficient length of contiguous nucleotides of SEQ ID NO:9 to identify the sequence as a fragment of the transgenic insert of sugar beet event Bv_CSM63713 in a sample of DNA.

Methods for designing and using primers and probes are well known in the art. DNA molecules comprising fragments of SEQ ID NOs:1-10 are useful as primers and probes for detecting sugar beet event Bv_CSM63713 and can readily be designed by one of skill in the art using the sequences provided herein. DNA probes and DNA primers are generally ten (10) nucleotides or more in length, often fifteen (15) nucleotides or more in length, twenty (20) nucleotides or more in length, or thirty (30) nucleotides or more in length. Such probes and primers are selected to be of sufficient length to hybridize specifically to a target sequence under stringency hybridization conditions.

Probes and primers may have complete sequence identity with the target sequence, although primers and probes differing from the target sequence that retain the ability to hybridize preferentially to target sequences may be designed by conventional methods. In order for a nucleic acid molecule to serve as a primer or probe it needs only be sufficiently complementary in sequence and/or of sufficient length to be able to form a stable double-stranded structure under the particular hybridization conditions or reaction conditions. Any conventional nucleic acid hybridization or amplification method can be used to identify the presence of transgenic DNA from sugar beet event Bv_CSM63713 in a sample. Polynucleotide molecules referred to as “polynucleotide segment of sufficient length” or “sufficient length of contiguous nucleotides” therefore are capable of specifically hybridizing to a target DNA sequence under certain hybridization conditions or reaction conditions. As used herein, the term “of sufficient length” refers to any length that is sufficient to be useful in a detection method of choice. Probes and primers are generally at least about 8 nucleotides, at least about 10 nucleotides, at least about 12 nucleotides, at least about 14 nucleotides, at least about 16 nucleotides, at least about 18 nucleotides, at least about 20 nucleotides, at least about 22 nucleotides, at least about 24 nucleotides, at least about 26 nucleotides, at least about 28 nucleotides, or at least about 30 nucleotides or more in length. Such probes and primers hybridize specifically to a target DNA sequence under stringent hybridization conditions. Conventional stringency conditions are described by MR Green and J Sambrook, Molecular cloning: a laboratory manual, 4^{th} Edition, Cold Spring Harbor Laboratory Press, Cold Spring Harbor, N.Y. (2012).

As used herein, two nucleic acid molecules are capable of specifically hybridizing to one another if the two molecules are capable of forming an anti-parallel, double-stranded nucleic acid structure. A nucleic acid molecule is the “complement” of another nucleic acid molecule if they exhibit complete complementarity. As used herein, two molecules exhibit “complete complementarity” if when aligned every nucleotide of the first molecule is complementary to every nucleotide of the second molecule. Two molecules are “minimally complementary” if they can hybridize to one another with sufficient stability to permit them to remain annealed to one another under at least conventional “low-stringency” conditions. Similarly, the molecules are “complementary” if they can hybridize to one another with sufficient stability to permit them to remain annealed to one another under conventional “high-stringency” conditions. Departures from complete complementarity are therefore permissible, as long as such departures do not completely preclude the capacity of the molecules to form a double-stranded structure.

As used herein, “stringent hybridization conditions” refers to conditions under which a polynucleotide will hybridize to its target sequence, typically in a complex mixture of nucleic acids, but to essentially no other sequences. “Stringent conditions” or “stringent hybridization conditions” when referring to a polynucleotide probe, refer to conditions under which a probe will hybridize to its target sequence to a detectably greater degree than to other sequences (e.g., at least 2-fold over background). Stringent conditions are sequence-dependent and will be different in different circumstances. Longer sequences hybridize specifically at higher temperatures. Generally, stringent conditions are selected to be about 5-10° C. lower than the thermal melting point (Tₘ) for the specific sequence at a defined ionic strength pH. The Tₘ is the temperature (under defined ionic strength, pH, and nucleic acid concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. By controlling the stringency of the hybridization and/or washing conditions, target sequences that are 100% complementary to the probe can be identified (homologous probing). Alternatively, stringency conditions can be adjusted to allow some mismatching in sequences so that lower degrees of identity are detected (heterologous probing).

As used herein, a substantially complementary or identical sequence is a polynucleotide that will specifically hybridize to the nucleic acid molecule to which it is being compared, or to its complement, respectively, under high stringency conditions. Appropriate stringency conditions which promote DNA hybridization, for example, 6× sodium chloride/sodium citrate (SSC) at about 450 C, followed by a wash of 2×SSC at 50° C., are known to those skilled in the art or can be found in Current Protocols in Molecular Biology, John Wiley & Sons, N.Y. (1989), 6.3.1-6.3.6.

A polynucleotide molecule of the present disclosure, such as a primer or probe, will specifically hybridize to at least one nucleic acid molecule selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, a polynucleotide having a nucleotide sequence that is at least 90%, at least 91%, at least 92%, at least 93%, at least 94%, at least 95%, at least 96%, at least 97%, at least 98%, at least 99%, 99.1%, at least 99.2%, at least 99.3%, at least 99.4%, at least 99.5%, at least 99.6%, at least 99.7%, at least 99.8%, at least 99.9% identical to SEQ ID NO:10, or a complete complement of or fragment of any of the foregoing under stringent hybridization conditions, or under moderately stringent hybridization conditions if the sequence of the polynucleotide molecule is not identical to the at least one of the nucleic acid molecules. An illustrative DNA sequence useful as a probe for detecting sugar beet event Bv_CSM63713 is provided as SEQ ID NO:36. The hybridization of the probe to the target DNA molecule can be detected by any number of methods known to those skilled in the art, which can include, but are not limited to, fluorescent tags, radioactive tags, antibody based tags, and chemiluminescent tags.

Regarding the amplification of a target polynucleotide (e.g., by PCR) using a particular amplification primer pair, “stringent conditions” or “stringent hybridization conditions” are conditions that permit the primer pair to hybridize to the target polynucleotide to which a primer having the corresponding wild-type sequence (or its complement) would bind and to produce an identifiable amplification product (the amplicon) having a sugar beet Bv_CSM63713 event specific region in a DNA thermal amplification reaction.

The term “specific for” a target sequence indicates that a probe or primer hybridizes under stringent hybridization conditions only to the target sequence in a sample comprising the target sequence.

Appropriate stringency conditions that promote DNA hybridization, for example, 6.0× sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6.3.6. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or either the temperature or the salt concentration may be held constant while the other variable is changed.

A diagnostic amplicon produced by the methods described herein may be detected by a plurality of techniques known in the art, such as sequencing, restriction mapping, Southern analysis, or any other suitable polynucleotide or DNA hybridization, blotting, polymerization and/or amplification-based approach or technique. One method is Genetic Bit Analysis (Nikiforov et al., 1994) where a DNA oligonucleotide is designed that overlaps both the adjacent flanking genomic DNA sequence and the inserted DNA sequence—i.e., a junction sequence. The oligonucleotide is immobilized in wells of a microtiter plate. Following PCR of the region of interest (using, for example, one primer in the inserted sequence and one in the adjacent flanking genomic sequence), a single-stranded PCR product can be hybridized to the immobilized oligonucleotide and serve as a template for a single base extension reaction using a DNA polymerase and labeled dideoxynucleotide triphosphates (ddNTPs) specific for the expected next base. Readout may be fluorescent or ELISA-based. A signal indicates presence of the transgene/genomic junction sequence due to successful amplification, hybridization, and single base extension.

Another method is the pyrosequencing technique as described by Winge (2000). In this method, an oligonucleotide is designed that overlaps the adjacent genomic DNA and insert DNA junction. The oligonucleotide is hybridized to single-stranded PCR product from the region of interest (one primer in the inserted sequence and one in the flanking genomic sequence) and incubated in the presence of a DNA polymerase, ATP, sulfurylase, luciferase, apyrase, adenosine 5′ phosphosulfate and luciferin. DNTPs are added individually and the incorporation results in a light signal that is measured. A light signal indicates the presence of the transgene/genomic sequence due to successful amplification, hybridization, and single or multi-base extension.

Fluorescence Polarization as described by Chen et al. (1999) is a method that can be used to detect the amplicon of the present invention. Using this method an oligonucleotide is designed that overlaps the genomic flanking and inserted DNA junction. The oligonucleotide is hybridized to single-stranded PCR product from the region of interest (one primer in the inserted DNA and one in the flanking genomic DNA sequence) and incubated in the presence of a DNA polymerase and a fluorescent-labeled ddNTP. Single base extension results in incorporation of the ddNTP. Incorporation can be measured as a change in polarization using a fluorometer. A change in polarization indicates the presence of the transgene/genomic sequence due to successful amplification, hybridization, and single base extension.

Real-time polymerase chain reaction (PCR) has the ability to monitor the progress of the PCR as it occurs (i.e., in real time). Data is collected throughout the PCR process, rather than at the end of the PCR. In real-time PCR, reactions are characterized by the point in time during cycling when amplification of a target is first detected rather than the amount of target accumulated after a fixed number of cycles. In a real-time PCR assay, a positive reaction is detected by accumulation of a fluorescent signal. The higher the starting copy number of the nucleic acid target, the sooner a significant increase in fluorescence is observed. The cycle threshold (Ct value) is defined as the number of cycles required for the fluorescent signal to cross the threshold (i.e., exceeds background level). Ct levels are inversely proportional to the amount of target nucleic acid in the sample (i.e., the lower the Ct value, the greater the amount of target nucleic acid in the sample).

Taqman® (PE Applied Biosystems, Foster City, CA) is described as a method of detecting and quantifying the presence of a DNA sequence using real-time PCR and is fully understood in the instructions provided by the manufacturer. Briefly, a FRET oligonucleotide probe is designed that overlaps the genomic flanking and insert DNA junction. The FRET probe and PCR primers (one primer in the insert DNA sequence and one in the flanking genomic sequence) are cycled in the presence of a thermal stable polymerase and dNTPs. Hybridization of the FRET probe results in cleavage and release of the fluorescent moiety away from the quenching moiety on the FRET probe. A fluorescent signal indicates the presence of the transgene/genomic sequence due to successful amplification and hybridization.

Molecular beacons have been described for use in sequence detection as described in Tyangi et al. (1996). Briefly, a FRET oligonucleotide probe is designed that overlaps the flanking genomic and insert DNA junction. The unique structure of the FRET probe results in it containing secondary structure that keeps the fluorescent and quenching moieties in close proximity. The FRET probe and PCR primers (one primer in the insert DNA sequence and one in the flanking genomic sequence) are cycled in the presence of a thermostable polymerase and dNTPs. Following successful PCR amplification, hybridization of the FRET probe to the target sequence results in the removal of the probe secondary structure and spatial separation of the fluorescent and quenching moieties. A fluorescent signal results and indicates the presence of the flanking/transgene insert sequence due to successful amplification and hybridization.

Other detection methods known in the art may be used. For example, microfluidics (see, e.g., U.S. Patent Publication No. 2006/068398; U.S. Pat. No. 6,544,734) provide methods and devices that can be used to separate and amplify DNA samples or molecules. Optical dyes can be used to detect and measure specific DNA molecules (see, e.g., WO/05017181). Nanotube devices (see, e.g., WO/06024023) that comprise an electronic sensor for the detection of DNA molecules or nanobeads that bind specific DNA molecules can then be detected. Nanopore sequencing technology, such as that described in Wang et al. (2021), Tyler et al. (2018), or Pearson et al. (2019), can also be used for event detection.

Provided are proteins that can be used to produce antibodies for detecting the presence of sugar beet event Bv_CSM63713 in a sample. Such antibodies are specific for one or more of the proteins that are encoded by sugar beet event Bv_CSM63713. Methods for preparing a polyclonal antibody or a monoclonal antibody are well-known to those skilled in the art, and can be used to make antibodies specific for one or more of the proteins encoded by sugar beet event Bv_CSM63713. For example, U.S. Pat. No. 7,838,729 and Wang et al. (2016) describe antibodies to DMO; Harrison et al. (1996) and Chinnadurai et al. (2018) describe antibodies to CP4 EPSPS. U.S. Pat. No. 9,371,394 describes antibodies to PAT enzyme. The DNA sequence encoding such proteins is provided in SEQ ID NO:10 and the start positions and stop positions of the coding sequences are indicated in Table 15 in Example 7. The DNA sequence encoding each protein and the protein encoded by the sequence are useful to produce antibodies for detecting the presence of sugar beet event Bv_CSM63713 by the methods described herein. Detection of the presence of sugar beet event Bv_CSM63713 may be done by using any protein detection techniques known in the art, such as Western blot analysis, immuno-precipitation, enzyme-linked immunosorbent assay (ELISA), antibody attachment to a detectable label or reporter molecule (such as a radioactive isotope, ligand, chemiluminescent agent, or enzyme), or enzymatic action on a reporter molecule. One method provides for contacting a sample with an antibody that binds to the PAT, DMO, or CP4-EPSPS protein encoded by sugar beet event Bv_CSM63713 and then detecting the presence or absence of antibody binding. The binding of such antibody is diagnostic for the presence of one or more proteins encoded by sugar beet event Bv_CSM63713.

Protein and nucleic acid detection kits for detecting the presence of sugar beet event Bv_CSM63713 are provided. Variations on such kits can also be developed using the compositions and methods disclosed herein and the methods well known in the art of protein and nucleic acid detection for identification of sugar beet event Bv_CSM63713. Protein and nucleic acid detection kits can be applied to methods for breeding with plants comprising sugar beet event Bv_CSM63713. Such kits contain primers and/or probes or antibodies which are specific to sugar beet event Bv_CSM63713. Such DNA primers and/or probes may comprise fragments of one or more of SEQ ID NOs:1-10, or antibodies specific for a protein encoded by sugar beet event Bv_CSM63713. The kits can also contain instructions for using the primers, probes, or antibodies for detecting the presence of sugar beet event Bv_CSM63713. Kits may optionally also comprise reagents for performing the detection or diagnostic reactions described herein.

One example of a detection kit comprises at least one DNA molecule of sufficient length of contiguous nucleotides of SEQ ID NO:10 to function as a DNA probe useful for detecting the presence or absence of sugar beet event Bv_CSM63713 in a sample. The DNA derived from transgenic sugar beet plants comprising event Bv_CSM63713 would comprise a DNA molecule having at least one sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10, a complement of any of the foregoing, or a fragment of any of the foregoing. An illustrative DNA molecule sufficient for use as a probe is one comprising the sequence provided as SEQ ID NO:36. Other probes may be readily designed by one of skill in the art. The probe can include a junction sequence that spans the 5′ or 3′ junction between the sugar beet genomic DNA and the transgenic insert of sugar beet event Bv_CSM63713.

Another example of a detection kit comprises at least one primer pair that specifically hybridize to a target DNA and amplify a diagnostic amplicon under the appropriate reaction conditions useful for detecting the presence or absence of sugar beet event Bv_CSM63713 in a sample. A kit that contains DNA primers that are homologous or complementary to any portion of the sugar beet genomic region as set forth in SEQ ID NO:10 and to any portion of the inserted transgenic DNA as set forth in SEQ ID NO:9 is an object of the present disclosure. The kit may provide an agarose gel-based detection method or any number of methods of detecting the amplicon(s) that are known in the art. Such a method may also include sequencing the amplicon or a fragment thereof. Illustrative DNA molecules sufficient for use as a primer pair are ones comprising the sequences provided as SEQ ID NO:19/20 and SEQ ID NO:23, SEQ ID NO:14/15 and SEQ ID NO:18, and SEQ ID NO:34 and SEQ ID NO:35, respectively, wherein the primer pair SEQ ID NO:19/20 and SEQ ID NO:23, SEQ ID NO:14/15 and SEQ ID NO:18, and SEQ ID NO:34 and SEQ ID NO:35 will produce an amplicon diagnostic for the presence of event Bv_CSM63713 in a sample. Other primer pairs may be readily designed by one of skill in the art.

Another example of a detection kit comprises at least one antibody specific for at least one protein encoded by sugar beet event Bv_CSM63713. For example, such a kit may utilize a lateral flow strip comprising reagents activated when the tip of the strip is contacted with an aqueous solution. Illustrative proteins sufficient for use in antibody production are ones encoded by the sequence provided as SEQ ID NO:10, or any fragment thereof. Detection of binding of the at least one antibody to the at least one protein encoded by sugar beet event Bv_CSM63713 in a sample is diagnostic for the presence of sugar beet event Bv_CSM63713 in the sample.

The detection kits provided herein are useful for, among other things, identifying sugar beet event Bv_CSM63713, selecting plant varieties or hybrids comprising sugar beet event Bv_CSM63713, detecting the presence of DNA derived from the transgenic sugar beet plant comprising sugar beet event Bv_CSM63713 in a sample, and monitoring samples for the presence and/or absence of sugar beet plants comprising event Bv_CSM63713, or plant parts derived from sugar beet plants comprising sugar beet event Bv_CSM63713.

Sugar beet plants, progeny, seeds, cells, and plant parts comprising sugar beet event Bv_CSM63713 are provided, as well as commodity products produced using these. These sugar beet plants, plant parts, plant cells, seeds, progeny plants and commodity products contain or comprise sugar beet event Bv_CSM63713 or are derived from a transgenic sugar beet plant, plant part, plant cell, seed, progeny plant or commodity product containing or comprising event Bv_CSM63713. The sugar beet plants, progeny, seeds, cells, plant parts, and commodity products contain or comprise a detectable amount of a polynucleotide comprising at least one junction sequence and/or heterologous transgenic insert sequence of sugar beet event Bv_CSM63713, such as a polynucleotide or nucleic acid or DNA molecule comprising at least one of the sequences provided as SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, a polynucleotide comprising at least 21 consecutive nucleotides of SEQ ID NO:1, at least 25 consecutive nucleotides of SEQ ID NO:2, at least 33 consecutive nucleotides of SEQ ID NO:3, at least 33 consecutive nucleotides of SEQ ID NO:4, at least 53 consecutive nucleotides of SEQ ID NO:5, or at least 51 consecutive nucleotides of SEQ ID NO:6, a nucleic acid molecule comprising a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or to the full length of SEQ ID NO:9, and a complete complement of any of the foregoing. In some embodiments, the sugar beet plant, plant part, plant cell, or seed is further defined as a progeny plant of any generation of a sugar beet plant comprising sugar beet event Bv_CSM63713, or a sugar beet plant part, plant seed, or plant cell derived therefrom.

The sugar beet plants, plant parts, plant cells, seeds, progeny plants and commodity products express or contain at least one herbicide tolerance gene selected from the group consisting of dicamba monooxygenase (DMO), phosphinothricin N-acetyltransferase (PAT), 5-enolpyruvylshikimate-3-phosphate synthase (EPSPS), and any combination thereof, and are tolerant to at least one herbicide selected from the group consisting of dicamba, glufosinate, glyphosate, and any combination thereof.

Also provided are sugar beet plants, plant seeds, plant parts, and plant cells tolerant to herbicides with three different herbicide modes of action, wherein the genes that confer the herbicide tolerance are present at a single genomic location. To make such a sugar beet plant, plant seed, plant part, or plant cell, three transgenic cassettes comprising herbicide tolerance genes can be inserted at a single genomic location within the sugar beet genome as a contiguous polynucleotide or a single molecularly linked transgenic insert. Alternatively, three transgene cassettes containing herbicide tolerance genes can be inserted at a single genomic location by inserting separate cassettes containing the herbicide tolerance genes at the same location. By “single genomic location” it is meant that the genes, together with any regulatory sequences (e.g., promoters, introns, leader sequences, 5′-UTRs, and/or 3′UTRs, etc.) and/or sequences encoding targeting peptides (e.g., chloroplast transit peptides) are present at a single location on a chromosome, and will be inherited as a single locus. While some intervening sequence may be present between each transgene cassette, the length of the intervening sequence is limited such that the transgenes cassettes are close to one another on the chromosome. For example, the intervening sequence between the transgene cassettes may be 500 nucleotides or fewer in length, 400 nucleotides or fewer in length, 300 nucleotides or fewer in length, 250 nucleotides or fewer in length, 200 nucleotides or fewer in length, 150 nucleotides or fewer in length, 100 nucleotides or fewer in length, or 50 nucleotides or fewer in length. For example, the sugar beet plant, plant seed, plant part, or plant cell can comprise any of DNA constructs described herein, and can exhibit tolerance to at least one herbicide selected from the group consisting of benzoic acid auxins such as dicamba, glutamine synthetase inhibitors such as glufosinate, EPSPS inhibitors such as glyphosate, and any combination thereof.

As used herein, the term “percent sequence identity” or “% sequence identity” refers to the percentage of identical nucleotides or amino acids in a linear polynucleotide or polypeptide sequence of a reference (“query”) sequence (or its complementary strand) as compared to a test (“subject”) sequence (or its complementary strand) when the two sequences are optimally aligned (with appropriate nucleotide or amino acid insertions, deletions, or gaps totaling less than 20 percent of the reference sequence over the window of comparison). Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and by computerized implementations of these algorithms such as GAP, BESTFIT, FASTA, and TFASTA available as part of the Sequence Analysis software package of the GCG® Wisconsin Package® (Accelrys Inc., San Diego, Calif.), MEGAlign (DNAStar Inc., 1228 S. Park St., Madison, Wis. 53715), and MUSCLE (version 3.6) (Edgar, “MUSCLE: multiple sequence alignment with high accuracy and high throughput” Nucleic Acids Research 32(5):1792-7 (2004)) for instance with default parameters. An “identity fraction” for aligned segments of a test sequence and a reference sequence is the number of identical components that are shared by the two aligned sequences divided by the total number of components in the portion of the reference sequence segment being aligned, that is, the entire reference sequence or a smaller defined part of the reference sequence. Percent sequence identity is represented as the identity fraction multiplied by 100. The comparison of one or more sequences may be to a full-length sequence or a portion thereof, or to a longer sequence. Sugar beet plants, progeny, seeds, cells, plant parts and commodity products comprising a detectable amount of a polynucleotide having a nucleotide sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or the full length of SEQ ID NO: 9 are within the scope of the present disclosure.

The present disclosure provides plants, progeny, seeds, plant cells, and plant parts such as roots, beets, pollen, anthers, ovaries, embryos, ovules, flowers, stems, leaves, microspores, protoplasts, and calluses derived from a transgenic sugar beet plant comprising event Bv_63713. A representative sample of seed comprising event Bv_63713 has been deposited according to the Budapest Treaty for the purpose of enabling the present disclosure. The ATCC repository has assigned the Accession No. PTA-127098 to event Bv_CSM63713-containing seed.

A microorganism is provided. The microorganism comprises a polynucleotide molecule having a nucleotide sequence of SEQ ID NO:9, or a nucleotide sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:9. An example of such a microorganism is an Agrobacterium cell. Another example of such a microorganism is an E. coli cell.

A plant cell is provided comprising a polynucleotide molecule as described herein. For example, a plant cell is provided having a nucleotide sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, a nucleic acid molecule comprising a polynucleotide having a nucleotide sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or the full length of SEQ ID NO:9 present in its genome.

Plant cells and microorganisms of the present disclosure are useful in many industrial applications, including but not limited to: (i) use as research tools for scientific inquiry or industrial research; (ii) use in culture for producing endogenous or recombinant carbohydrate, lipid, nucleic acid, enzymes or protein products or small molecules that may be used for subsequent scientific research or as industrial products; and (iii) for the plant cells of the present disclosure, use with modem plant tissue culture techniques to produce transgenic plants or plant tissue cultures that may then be used for agricultural research or production. The production and use of such transgenic plant cells utilize modern microbiological techniques and human intervention to produce a man-made, unique plant cell. In this process, a recombinant DNA is inserted into a plant cell's genome to create a transgenic plant cell that is separate and unique from naturally occurring plant cells. This transgenic plant cell can then be cultured much like bacteria and yeast cells using modern microbiology techniques and may exist in an undifferentiated, unicellular state. The new plant cell's genetic composition and phenotype is a technical effect created by the integration of a heterologous DNA into the genome of the cell.

Provided are methods of using a plant cell, such as a transgenic plant cell. These include (i) methods of producing transgenic cells by integrating a recombinant DNA into the genome of the cell and then using this cell to derive additional cells possessing the same heterologous DNA; (ii) methods of culturing cells that contain recombinant DNA using modem microbiology techniques; (iii) methods of producing and purifying endogenous or recombinant carbohydrate, lipid, nucleic acid, enzymes or protein products from cultured cells; and (iv) methods of using modem plant tissue culture techniques with transgenic plant cells to produce transgenic plants or transgenic plant tissue cultures.

Plants, progeny, seeds, cells, and plant parts may also contain one or more additional desirable trait(s). Such desirable traits may be transgenic traits, native traits, or traits produced by other methods such as genome editing, base editing, prime editing or other conventional mutagenesis methods. Desirable traits may be combined with sugar beet event Bv_CSM63713 by, for example, crossing a sugar beet plant comprising sugar beet event Bv_CSM63713 with another sugar beet plant containing the additional trait(s), or transgenic events. Such traits or transgenic events include, but are not limited to, increased insect resistance, increased water use efficiency, increased yield performance, increased drought resistance, increased disease resistance, increased seed quality, improved nutritional quality, hybrid seed production, and/or increased herbicide tolerance, in which the trait is measured with respect to a sugar beet plant lacking such transgenic trait.

The plants described herein can be used to produce progeny that comprises sugar beet event Bv_CSM63713. As used herein, “progeny” includes any plant, seed, and cell and/or regenerable plant part comprising sugar beet event Bv_CSM63713 inherited from an ancestor plant, indicated by the plant comprising a polynucleotide molecule having at least one polynucleotide sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, a polynucleotide comprising at least 21 consecutive nucleotides of SEQ ID NO:1, at least 25 consecutive nucleotides of SEQ ID NO:2, at least 33 consecutive nucleotides of SEQ ID NO:3, at least 33 consecutive nucleotides of SEQ ID NO:4, at least 53 consecutive nucleotides of SEQ ID NO:5, or at least 51 consecutive nucleotides of SEQ ID NO:6, or a polynucleotide having a nucleotide sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or the full length of SEQ ID NO: 9.

Plants, seeds, progeny, plant parts and cells may be homozygous or heterozygous for sugar beet event Bv_CSM63713. Progeny plants may be grown from seeds produced by a sugar beet plant comprising sugar beet event Bv_CSM63713 or from seeds produced by a sugar beet plant fertilized with pollen containing sugar beet event Bv_CSM63713.

Progeny plants may be self-pollinated (also known as “selfing”) to generate a true breeding line of plants, i.e., plants homozygous for the sugar beet event Bv_CSM63713 DNA. Alternatively, progeny plants may be outcrossed, i.e., bred with another plant, to produce a varietal or a hybrid seed or plant. The other plant may be transgenic or nontransgenic. A varietal or hybrid seed or plant of the present disclosure may thus be derived by crossing a first parent that lacks the specific and unique DNA of event Bv_CSM63713 with a second parent comprising event Bv_CSM63713, resulting in a hybrid comprising the specific and unique DNA of event Bv_CSM63713.

A variety or hybrid seed or plant of the present disclosure may also be produced through a three-way cross (Poehlman J. M., 1987). In such a case, three parental lines are used in crossing: a first parental line, a second parent line and a pollinator. The F1 line produced by crossing a first parent comprising event Bv_CSM63713 with a second parent also comprising event Bv_CSM63713 is crossed with a third parent that lacks the specific and unique DNA of event Bv_CSM63713. The parent comprising event Bv_CSM63713 for hybrid seed or plant may be the maternal or the paternal parent. Preferably, the parent comprising event Bv_CSM63713 is the maternal parent as this parent is cytoplasmic male sterile and does not produce transgenic pollen during varietal or hybrid seed production in sugar beet.

Each parent can be a hybrid or an inbred/variety, so long as the cross or breeding results in a plant or seed of the present disclosure, i.e., a seed having at least one allele comprising the specific and unique DNA of event Bv_CSM63713 and/or at least 21 consecutive nucleotides of SEQ ID NO:1, at least 25 consecutive nucleotides of SEQ ID NO:2, at least 33 consecutive nucleotides of SEQ ID NO:3, at least 33 consecutive nucleotides of SEQ ID NO:4, at least 53 consecutive nucleotides of SEQ ID NO:5, at least 51 consecutive nucleotides of SEQ ID NO:6, or a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full-length of SEQ ID NO:10 or the full length of SEQ ID NO: 9.

Sexually crossing one plant with another plant, i.e., cross-pollinating, may be accomplished or facilitated by human intervention, for example: by human hands collecting the pollen of one plant and contacting this pollen with the style or stigma of a second plant; by human hands and/or human actions removing, destroying, or covering the stamen or anthers of a plant (e.g., by manual intervention or by application of a chemical gametocide) so that natural self-pollination is prevented and cross-pollination would have to take place in order for fertilization to occur; by human placement of pollinating insects in a position for “directed pollination” (e.g., by placing beehives in orchards or fields or by caging plants with pollinating insects); by human opening or removing of parts of the flower to allow for placement or contact of foreign pollen on the style or stigma; by selective placement of plants (e.g., intentionally planting plants in pollinating proximity); and/or by application of chemicals to precipitate flowering or to foster receptivity (of the stigma for pollen).

A plant part is provided. As used herein, a “plant part” refers to any part of a plant that is comprised of material directly from or derived from a plant comprising sugar beet event Bv_CSM63713. Plant parts include but are not limited to roots, beets, pollen, anthers, ovaries, ovules, embryos, flowers, stems, leaves, microspores, protoplasts, and calluses, in whole or part. Plant parts may be viable or nonviable, regenerable and/or non-regenerable.

Commodity products that are produced from plants comprising sugar beet event Bv_CSM63713 are provided. The commodity products contain a detectable amount of DNA comprising a DNA sequence selected from the group consisting of SEQ ID NOs:1-10, or a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or the full length of SEQ ID NO:9. As used herein, a “commodity product” refers to any composition or product which is comprised of material from plant, seed, cell, or plant part comprising sugar beet event Bv_CSM63713. Commodity products may be viable or non-living plant material, that is, a material that is not living and derived from a plant, seed, cell, or plant part comprising sugar beet event Bv_CSM63713. Nonviable commodity products include but are not limited to nonviable seeds, whole or processed seeds, processed plant tissues or plant parts, dehydrated plant tissues or parts, frozen plant tissues or parts, plant parts processed for animal feed, fiber, pulp pellets, pulp shreds, tailing, juice, syrup, molasses, extract, raffinate, betaine, separator molasses solubles (SMS), or any other food for human consumption. Viable commodity products include but are not limited to viable seeds, viable plant parts (such as root and leaf) and viable plant cells. A plant comprising event Bv_CSM63713 can thus be used to manufacture any commodity product typically acquired from a sugar beet plant. Any such commodity product that is derived from the plants comprising event Bv_CSM63713 may contain at least a detectable amount of the specific and unique DNA corresponding to event Bv_CSM63713, and specifically may contain a detectable amount of a polynucleotide having a nucleotide sequence of at least 21 consecutive nucleotides of SEQ ID NO:1, at least 25 consecutive nucleotides of SEQ ID NO:2, at least 33 consecutive nucleotides of SEQ ID NO:3, at least 33 consecutive nucleotides of SEQ ID NO:4, at least 53 consecutive nucleotides of SEQ ID NO:5, or at least 51 consecutive nucleotides of SEQ ID NO:6, or a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or the full length of SEQ ID NO:9. Any standard method of detection for polynucleotide molecules may be used, including methods of detection disclosed herein.

A plant tolerant to herbicides may be produced by sexually crossing a plant comprising event Bv_CSM63713 comprising a polynucleotide having the nucleotide sequence of SEQ ID NOs:1-10, at least 21 consecutive nucleotides of SEQ ID NO:1, at least 25 consecutive nucleotides of SEQ ID NO:2, at least 33 consecutive nucleotides of SEQ ID NO:3, at least 33 consecutive nucleotides of SEQ ID NO:4, at least 53 consecutive nucleotides of SEQ ID NO:5, or at least 51 consecutive nucleotides of SEQ ID NO:6, and a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or the full length of SEQ ID NO:9, with another plant and thereby producing seed, which is then grown into progeny plants. These progeny plants may be analyzed using diagnostic methods to select for progeny plants that comprise event Bv_CSM63713 DNA or for progeny plants tolerant to herbicides dicamba, glyphosate, glufosinate, and any combination thereof. The other plant used may or may not be transgenic. The progeny plant and/or seed produced may be varietal or hybrid seed.

A plant tolerant to herbicides may be produced by selfing a plant comprising event Bv_CSM63713 comprising a polynucleotide having the nucleotide sequence of SEQ ID NOs:1-10, at least 21 consecutive nucleotides of SEQ ID NO:1, at least 25 consecutive nucleotides of SEQ ID NO:2, at least 33 consecutive nucleotides of SEQ ID NO:3, at least 33 consecutive nucleotides of SEQ ID NO:4, at least 53 consecutive nucleotides of SEQ ID NO:5, or at least 51 consecutive nucleotides of SEQ ID NO:6, and a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or the full length of SEQ ID NO:9, and thereby producing seed, which is then grown into progeny plants. These progeny plants may then be analyzed using diagnostic methods to select for progeny plants that comprise event Bv_CSM63713 DNA, or for progeny plants tolerant to herbicides dicamba, glyphosate, glufosinate, and any combination thereof. Sugar beet event Bv_CSM63713 contains three expression cassettes that together provide tolerance to benzoic acid auxins; inhibitors of 5-enolpyruvylshikimate-3-phosphate synthase (EPSPS); and inhibitors of glutamine synthetase.

Sugar beet event Bv_CSM63713 contains three expression cassettes that together provide tolerance to benzoic acid auxins such as dicamba; inhibitors of glutamine synthetase such as glufosinate, and inhibitors of EPSPS such as glyphosate.

As used herein, inhibitors of glutamine synthetase include, but are not limited to, phosphinothricin, glufosinate, glufosinate salts, glufosinate-ammonium, glufosinate-sodium, glufosinate-P, L-glufosinate-ammonium, and L-glufosinate-sodium.

As used herein, benzoic acid herbicides include, but are not limited to, dicamba (3,6-dichloro-2-methoxybenzoic acid), dicamba salts, dicamba-butotyl, dicamba-diglycolamine salt, dicamba-dimethylammonium, dicamba-diethanolammonium, dicamba-isopropylammonium, dicamba-potassium, dicamba-sodium, and dicamba-trolamine.

As used herein, inhibitors of 5-enolpyruvylshikimate-3-phosphate synthase (EPSPS) include, but are not limited to, glyphosate, glyphosate salts, glyphosate-isopropylammonium, glyphosate-ammonium, glyphosate-dimethylammonium, glyphosate-trimesium (=sulfosate), glyphosate-diammonium, glyphosate-potassium, and glyphosate-sodium.

As used herein, “herbicide tolerant” or “herbicide tolerance” or “tolerance” means the ability to be wholly or partially unaffected by the presence or application of one of more herbicide(s), for example to resist the toxic effects of an herbicide when applied. A cell, seed, or plant is “herbicide tolerant” or has “improved tolerance” if it can maintain at least some normal growth or phenotype in the presence of one or more herbicide(s). A trait is an herbicide tolerance trait if its presence can confer improved tolerance to an herbicide upon a cell, plant, or seed as compared to the wild-type or control cell, plant, or seed. Crops comprising an herbicide tolerance trait can continue to grow in the presence of the herbicide and may be minimally affected by the presence of the herbicide. A protein confers “herbicide tolerance” if expression of the protein can confer improved tolerance to an herbicide upon a cell, plant, or seed as compared to the wild-type or control cell, plant, or seed. Examples of herbicide tolerance proteins are phosphinothricin N-acetyltransferase, dicamba monooxygenase, and glyphosate tolerant 5-enolpyruvylshikimate-3-phosphate synthase from Agrobacterium sp strain CP4. Herbicide tolerance may be complete or partial insensitivity to a particular herbicide and may be expressed as a percent (%) tolerance or insensitivity to a particular herbicide.

As used herein “herbicide injury” or “injury” refers to injury to a plant because of the application of an herbicide. The “injury rate” or “percent injury” refers to a visual evaluation of injury caused by an herbicide, and is expressed as the percentage of leaf area of a plant exhibiting damage such as necrosis (brown or dead tissue), chlorosis (yellow tissue or yellow spotting) and malformation (misshapen leaves or plant structures, epinasty or twisting of stem, cupping of leaves) caused by herbicide application based on the visual evaluation. It is measured on a scale of 0 to 100, where “0” representing no crop injury and “100” denoting complete crop injury (death). For sugar beet plants containing sugar beet event Bv_CSM63713, the plant will have decreased injury after application of one or more of: a benzoic acid auxin (such as dicamba); an inhibitor of 5-enolpyruvylshikimate-3-phosphate synthase (EPSPS) (such as glyphosate); or an inhibitor of glutamine synthetase (such as glufosinate); or any of the combinations thereof. For example, sugar beet plants containing sugar beet event Bv_CSM63713 will have less than about 5% injury, less than about 10% injury, less than about 15% injury, or less than about 20% injury following application of a benzoic acid auxin such as dicamba, an EPSPS inhibitor such as glyphosate, or a glutamine synthetase inhibitor such as glufosinate, as compared to otherwise identical sugar beet plants that do not contain sugar beet event Bv_CSM63713.

As used herein, a “weed” is any undesired plant. A plant may be considered generally undesirable for agriculture or horticulture purposes (for example, Amaranthus species) or may be considered undesirable in a particular situation (for example, a crop plant of one species in a field of a different species, also known as a volunteer plant). Weeds are commonly known in the art and vary by geography, season, growing environment, and time. Lists of weed species are available from agricultural and scientific societies and efforts (such as the Weed Science Society of America, the Canadian Weed Science Society, the Brazilian Weed Science Society, the International Weed Science Society, and the International Survey of Herbicide Resistant Weeds), government agencies (such as the United States Department of Agriculture and the Australia Department of the Environment and Energy), and industry and farmer associations (such as the American Sugarbeet Growers Association). Major troublesome weeds in sugar beet production include waterhemp (Amaranthus tuberculatus (Moq.) J. D. Sauer), kochia (Bassia scoparia (L.) A. J. Scott or Kochia scoparia (L.)), common lambsquarters (Chenopodium album L.), horseweed/marestail (Erigeron canadensis L.), palmer amaranth (Amaranthus palmeri), redroot pigweed (Amaranthus retroflexus L.), velvetleaf (Abutilon theophrasti Medik.), and yellow nutsedge (Cyperus esculentus L.) (Soltani et al., 2018).

Methods for controlling weeds in an area for sugar beet cultivation are provided. The methods comprise applying at least one herbicide selected from the group consisting of (i) inhibitors of glutamine synthetase such as glufosinate; (ii) benzoic acid auxins such as dicamba; and (iii) the 5-enolpyruvylshikimate-3-phosphate synthase (EPSPS) inhibitor glyphosate, where seeds or plants comprising sugar beet event Bv_CSM63713 are planted in the area before, at the time of, or after applying the herbicide and the herbicide application prevents or inhibits weed growth and does not injure the sugar beet plants. The plant growth area may or may not comprise weed seeds or plants at the time of herbicide application. The herbicide(s) used in the methods described herein can be applied alone or in combination with one or more herbicide(s) during the growing season. The herbicide(s) used in the methods described herein can be applied in combination with one or more herbicide(s) temporally (for example, as a tank mixture or in sequential applications), spatially (for example, at different times during the growing season including before and after sugar beet seed planting), or both. For example, a method for controlling weeds is provided that comprises planting seed comprising sugar beet event Bv_CSM63713 in an area and applying an herbicidally effective amount over the growing season of one or more of dicamba, glyphosate or glufosinate, alone or in any combination with another herbicide, for the purpose of controlling weeds in the area with no injury or less than about 10% injury to the plants containing sugar beet event Bv_CSM63713. Such application of herbicide(s) may be pre-planting (any time prior to planting seed comprising sugar beet event Bv_CSM63713, including for burn-down purposes, that is application to emerging or existing weeds prior to seed plant), pre-emergence (any time after seed comprising sugar beet event Bv_CSM63713 is planted and before plants comprising sugar beet event Bv_CSM63713 emerge), or post-emergence (any time after plants comprising sugar beet event Bv_CSM63713 emerge). Multiple applications of one or more herbicides, or a combination of herbicides together or individually, may be used over a growing season, for example, two applications (such as a pre-planting application and a post-emergence application, or a pre-emergence application and a post-emergence application) or three or more applications (such as a pre-planting application and two post-emergence applications).

Herbicide application in practicing the methods described herein may be at the recommended commercial rate or any fraction or multiple thereof, such as twice the recommended commercial rate. Herbicide rates may be expressed as pounds acid equivalent per acre (lb ae/acre), pounds active ingredient per acre (lb ai/acre) or pounds active ingredient per hectare (lb ai/ha), depending on the herbicide and the formulation. The use of acres in the herbicide application rates as provided herein is merely instructive; herbicide application rates in the equivalent dosages to any rate provided herein may be used for areas larger or smaller than an acre. The herbicide application comprises at least one herbicide selected from the group consisting of (i) inhibitors of glutamine synthetase such as glufosinate; (ii) benzoic acid auxins such as dicamba; and (iii) the 5-enolpyruvylshikimate-3-phosphate synthase (EPSPS) inhibitor glyphosate. The plant growth area may or may not comprise weed plants at the time of herbicide application. An herbicidally effective amount of glutamine synthetase inhibitors for use in the area for controlling weeds ranges from about 0.1 lb ae/acre to as much as about 10 lb ae/acre over a growing season (for example, glufosinate could be applied at a rate of about 0.4 lb ai/acre to about 2.16 lb ai/acre). An herbicidally effective amount of a benzoic acid auxin herbicide for use in the area for controlling weeds ranges from about 0.1 lb ae/acre to as much as about 16 lb ae/acre over a growing season (for example, dicamba could be applied at a rate of about 0.5 lb ae/acre to about 2.0 lb ae/acre). An herbicidally effective amount of EPSPS inhibitors for use in the area for controlling weeds ranges from about 0.5 lb ae/ac to about 12 lb ae/ac over a growing season (for example, glyphosate could be applied at a rate of about 0.75 lb ae/acre to about 2.25 lb ae/acre).

Methods for controlling volunteer sugar beet comprising sugar beet event Bv_CSM63713 in an area for crop cultivation are provided. The methods comprise applying an herbicidally effective amount of at least one herbicide, such as paraquat, clethodim, clopyralid, desmedipham, triflusulfuron, 2,4-dichliorophenoxyacetic acid (2, 4-D), and acetolactate synthase (ALS) inhibitors such as sulfonylureas (SUs), imidazolinones, triazolopyrimidines, pyrimidinyl oxybenzoates, and sulfonylarnino carbonyl triazolinones, and any combination thereof, where the herbicide application prevents growth of sugar beet comprising sugar beet event Bv_CSM63713.

Methods for producing plants and seeds comprising sugar beet event Bv_CSM63713 are provided. Plants may be bred using any method known in the art. For example, descriptions of breeding methods that are commonly used can be found in WR Fehr, in Breeding Methods for Cultivar Development, Wilcox J. ed., American Society of Agronomy, Madison WI (1987). Plants may be self-pollinated (also known as “selfing”) or cross-pollinated (also known as “crossing”). Plants comprising sugar beet event Bv_CSM63713 may be self-pollinated to generate a true breeding line of plants that are homozygous for sugar beet event Bv_CSM63713. Selfing results in progeny known as “inbred” and can be used to produce inbred lines that are genetically uniform. Alternatively, plants comprising sugar beet event Bv_CSM63713 may be cross-pollinated (bred with another plant that is transgenic or nontransgenic) to produce a varietal or a hybrid seed. As described further herein, sugar beet event Bv_CSM63713 comprises three independent expression cassettes or transgenes encoding a dicamba monooxygenase (DMO), a phosphinothricin N-acetyltransferase (PAT), and EPSPS, respectively. Transgenic sugar beet plant(s) comprising the event Bv_CSM63713 is/are tolerant to benzoic acid auxins such as dicamba, inhibitors of glutamine synthetase such as glufosinate, inhibitors of EPSPS such as glyphosate, or any combinations thereof, relative to a non-transgenic control plant. Transgenic sugar beet plants used in these methods may be homozygous or heterozygous for the transgenes. Progeny plants produced by these methods may be varietal or hybrid plants; may be grown from seeds produced by sugar beet event Bv_CSM63713 containing plant and/or from seeds produced by a plant fertilized with pollen from a sugar beet event Bv_CSM63713 containing plant; and may be homozygous or heterozygous for the transgenes and/or event Bv_CSM63713.

Furthermore, plants comprising sugar beet event Bv_CSM63713 may be generated through a three-way cross by crossing a first parental line comprising event Bv_CSM63713 with a second parent line also comprising event Bv_CSM63713 to produce an F1 line, which is then crossed with a third parent that lacks the specific and unique DNA of event Bv_CSM63713. The parent comprising event Bv_CSM63713 for hybrid seed or plant may be the maternal or the paternal parent. Preferably, the parent comprising event Bv_CSM63713 is the maternal parent as this parent is cytoplasmic male sterile and does not produce transgenic pollen during varietal or hybrid seed production.

The production of double haploids may also be used to produce sugar beet plants and seeds homozygous for event Bv_CSM63713 DNA in a breeding program. Double haploids are produced by the doubling of a set of chromosomes (1 N) from a heterozygous plant to produce a completely homozygous individual. For example, see Wan, et al., (1989) and U.S. Pat. No. 7,135,615. This can be advantageous because the process omits the generations of selfing needed to obtain a homozygous plant from a heterozygous source. One way of producing haploid and double haploid sugar beet plant comprising event Bv_CSM63713 is through ovule culture of unfertilized flowers comprising event Bv_CSM63713 (Gurel et al., 2021; Weich and Levall, 2003). Other methods such as natural polyembryony, induction with irradiated pollen, crosses with polyploid plants or wild species, and anther and microspore culture can also be applied to produce haploid and double haploid sugar beet plants comprising event Bv_CSM63713.

Seed and progeny plants made by the methods described herein comprise sugar beet event Bv_CSM63713. Application of one or more herbicide to which sugar beet event Bv_CSM63713 confers tolerance may be used to select progeny that comprise sugar beet event Bv_CSM63713. Alternatively, progeny may be analyzed using diagnostic methods to select for plants or seeds comprising sugar beet event Bv_CSM63713. Progeny may be varietal or hybrid plants; may be grown from seeds produced by a plant comprising sugar beet event Bv_CSM63713 or from seeds produced by a plant fertilized with pollen from a plant comprising sugar beet event Bv_CSM63713; and may be homozygous or heterozygous for sugar beet event Bv_CSM63713.

Sugar beet transgenic events are known to one of skill in the art; for example, a list of such traits is provided by the United States Department of Agriculture's (USDA) Animal and Plant Health Inspection Service (APHIS) and can be found on their website at www.aphis.usda.gov. Two or more transgenic events may thus be combined in a progeny seed or plant by crossing two parent plants each comprising one or more transgenic event(s), collecting progeny seed, and selecting for progeny seed or plants that contain the two or more transgenic events; these steps may then be repeated until the desired combination of transgenic events in a progeny is achieved. Back-crossing to a parental plant and out-crossing with a non-transgenic plant are also contemplated, as is vegetative propagation.

As used herein, the term “comprising” means “including but not limited to”.

Methods of detecting the presence of DNA derived from a sugar beet plant, plant part, plant cell, or seed comprising sugar beet event Bv_CSM63713 in a sample are provided. One method comprises (i) extracting a DNA sample from at least one sugar beet plant, plant part, plant cell, or seed; (ii) contacting the DNA sample with at least one primer that is capable of producing DNA sequence specific to event Bv_CSM63713 DNA under conditions appropriate for DNA sequencing; (iii) performing a DNA sequencing reaction; and then (iv) confirming that the nucleotide sequence comprises a nucleotide sequence specific for event Bv_CSM63713, of the transgenic insert comprised therein, such as one selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10.

Another method comprises (i) extracting a DNA sample from at least one sugar beet plant, plant part, plant cell, or seed; (ii) contacting the DNA sample with a primer pair that is capable of producing an amplicon from event Bv_CSM63713 DNA under conditions appropriate for DNA amplification; (iii) performing a DNA amplification reaction; and then (iv) detecting the amplicon molecule and/or confirming that the nucleotide sequence of the amplicon comprises a nucleotide sequence specific for event Bv_CSM63713, such as one selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10. The amplicon should be one that is specific for event Bv_CSM63713, and comprises the junction at nucleotide positions 1000-1001, and/or nucleotide positions 12,722-12,723 of SEQ ID NO:10, such as an amplicon that comprises SEQ ID NO:1, or SEQ ID NO:2, or SEQ ID NO:3, or SEQ ID NO:4, or SEQ ID NO:5, or SEQ ID NO:6, or SEQ ID NO:7, or SEQ ID NO:8, or SEQ ID NO:9, or SEQ ID NO:10. The detection of a nucleotide sequence specific for event Bv_CSM63713 in the amplicon is determinative and/or diagnostic for the presence of the sugar beet event Bv_CSM63713 specific DNA in the sample. An illustrative primer pair that is capable of producing an amplicon from event Bv_CSM63713 DNA under conditions appropriate for DNA amplification is provided as SEQ ID NO:14 and SEQ ID NO:18. Other primer pairs may be readily designed by one of skill in the art to produce an amplicon diagnostic for sugar beet event Bv_CSM63713, wherein such a primer pair comprises at least one primer within the genomic region flanking the insert and a second primer within the insert, provided that any primer pair could be designed and used that produces an amplicon comprising a junction sequence and/or all or part of the insert or transgene sequence. Detection of an amplicon could be based on any suitable method, such as sequencing, determining fragment size or migration of the amplicon in a matrix or gel, or a hybridization-based method.

Another method of detecting the presence of DNA derived from a sugar beet plant, plant part, plant cell, or seed comprising sugar beet event Bv_CSM63713 in a sample comprises (i) extracting a DNA sample from at least one sugar beet plant, plant part, plant cell, or seed; (ii) contacting the DNA sample with a DNA probe specific for event Bv_CSM63713 DNA; (iii) allowing the probe and the DNA sample to hybridize under stringent hybridization conditions; and then (iv) detecting hybridization between the probe and the target DNA sample. An example of the sequence of a DNA probe that is specific for event Bv_CSM63713 is provided as SEQ ID NO:36. Other probes may be readily designed by one of skill in the art. Detection of probe hybridization to the DNA sample is diagnostic for the presence of sugar beet event Bv_CSM63713 specific DNA in the sample. Absence of hybridization is alternatively diagnostic of the absence of sugar beet event Bv_CSM63713 specific DNA in the sample.

Methods for determining the zygosity of the event and transgene with genomic DNA derived from at least one sugar beet plant, plant part, plant cell or seed comprising sugar beet event Bv_CSM63713 in a sample are provided. One method comprising of (i) extracting a DNA sample from at least sugar beet plant, plant part, plant cell or seed; (ii) contacting the DNA sample with a first primer pair that is capable of producing a first amplicon diagnostic for event Bv_CSM63713; (iii) contacting the DNA sample with a second primer pair that is capable of producing a second amplicon diagnostic for wild-type genomic DNA not comprising event Bv_CSM63713; (iv) performing a DNA amplification reaction; and then (v) detecting the amplicons, wherein the presence of only the first amplicon is diagnostic of a homozygous event Bv_CSM63713 DNA in the sample, the presence of both the first amplicon and the second amplicon is diagnostic of a sugar beet plant heterozygous for event Bv_CSM63713, and the presence of only the second amplicon is diagnostic for the absence of event Bv_CSM63713 DNA in the sample. Illustrative sets of primer pairs are presented as SEQ ID NO:26 and SEQ ID NO:25, which produce an amplicon diagnostic for event Bv_CSM63713; and SEQ ID NO:26 and SEQ ID NO:24, which produce an amplicon diagnostic for wild-type sugar beet genomic DNA not comprising event Bv_CSM63713. A set of probes can also be incorporated into such an amplification method to be used in a real-time PCR format using the primer pair sets described above.

Another method for determining zygosity comprises (i) extracting a DNA sample from at least one sugar beet plant, plant part, plant cell or seed; (ii) contacting the DNA sample with a probe set which contains at least a first probe that specifically hybridizes to event Bv_CSM63713 DNA and at least a second probe that specifically hybridizes to sugar beet genomic DNA that was disrupted by insertion of the heterologous DNA of event Bv_CSM63713 and does not hybridize to event Bv_CSM63713 DNA; (iii) hybridizing the probe set with the sample under stringent hybridization conditions, wherein detecting hybridization of only the first probe under the hybridization conditions is diagnostic for a homozygous sugar beet plant, plant part, plant cell or seed for event Bv_CSM63713 DNA in the sample; wherein detecting hybridization of both the first probe and the second probe under the hybridization conditions is diagnostic for a heterozygous sugar beet plant, plant part, plant cell or seed for event Bv_CSM63713 in a DNA sample; and detecting hybridization of only the second probe under the hybridization conditions is diagnostic for the absence of event Bv_CSM63713 DNA in the sample.

Yet another method for determining zygosity comprises (i) extracting a DNA sample from at least one sugar beet plant, plant part, plant cell or seed; (ii) contacting the DNA sample with a first primer pair that is capable of producing a first amplicon diagnostic for event Bv_CSM63713; (iii) contacting the DNA sample with a second primer pair that is capable of producing a second amplicon of an internal standard known to be single-copy and homozygous in the sugar beet plant; (iv) contacting the DNA sample with a probe set which contains at least a first probe that specifically hybridizes to the first amplicon, and at least a second probe that specifically hybridizes to the second amplicon; (v) performing a DNA amplification reaction using real-time PCR and determining the cycle thresholds (Ct values) of the first and second amplicons; (vi) calculating the difference (ΔCt) between the Ct value of the first amplicon and the second amplicon; and (vii) determining zygosity, wherein a ΔCt of about zero (0) indicates homozygosity of the event or inserted T-DNA, and a ΔCt of about one (1) indicates heterozygosity of the event or inserted T-DNA. Heterozygous and homozygous events are differentiated by a ΔCt value unit of approximately one (1). Given the normal variability observed in real-time PCR due to multiple factors such as amplification efficiency and ideal annealing temperatures, the range of “about one (1)” is defined as a ΔCt of 0.75 to 1.25, and the range of “about zero (0)” is defined as a Act of −0.25 to 0.25 (or of 0.0 to 0.25 if the ΔCt is measured as an absolute value). Primer pairs and probes for the above method for determining zygosity can amplify and detect amplicons from the transgene or event DNA and the internal DNA standard.

A DNA construct is provided comprising a first expression cassette, a second expression cassette, and a third expression cassette, wherein the first expression cassette comprises in operable linkage i) promoter and leader sequences for the chlorophyll A-B (Cab1) binding protein gene from Arabidopsis thaliana, ii) a codon-optimized phosphinothricin N-acetyltransferase (PAT) coding sequence from Streptomyces viridochromogene for conferring tolerance to glutamine synthetase inhibitors such as glufosinate; and iii) a 3′ UTR (untranslated region) for a small heat shock protein (Hsp20) gene from Medicago truncatula; the second expression cassette comprises in operable linkage i) an enhancer for inclusion body matrix protein from Dahlia Mosaic Virus, ii) promoter, leader and intron sequences for a ubiquitin protein gene from Cucumis melo, iii) a chloroplast targeting sequence for a ribulose bisphosphate carboxylase small subunit (RbcS) gene from Pisum sativum, iv) a codon-optimized dicamba monooxygenase coding sequence (DMO) from Stenotrophomonas maltophilia for conferring tolerance to benzoic acid auxins such as dicamba, and v) a 3′ UTR for a putative protein gene from Medicago truncatula; the third expression cassette comprises in operable linkage i) promoter, leader and intron sequences for an S-adenosyl-L-methionine synthetase (SAMS2) gene from Cucumis melo, ii) a chloroplast targeting sequence for 5-enolpyruvylshikimate-3-phosphate synthase (EPSPS) gene from Arabidopsis thaliana; iii) a codon optimized coding sequence for 5-enolpyruvylshikimate-3-phosphate synthase (EPSPS) from Agrobacterium sp. strain CP4 for conferring tolerance to EPSPS inhibitors such as glyphosate; and iv) a 3′ UTR for a hypothetical protein from Medicago truncatula. The nucleotide sequences of the three expression cassettes were comprised in SEQ ID NO:9 and SEQ ID NO:10 of sugar beet event Bv_CSM63713. Expression of the DMO, PAT, and EPSPS in transgenic plants confers tolerance to herbicides with three different modes of action. For example, plants, plant parts, plant cells or seeds containing or comprising sugar beet event Bv_CSM63713 are tolerant to dicamba (a benzoic acid herbicide), glufosinate (a glutamine synthetase inhibitor), and glyphosate (an EPSPS inhibitor).

Methods of improving tolerance to herbicides are provided. The methods consist of i) inserting the DNA construct comprising a first expression cassette, a second expression cassette and a third expression cassette, as described herein, into the genome of a plant cell, ii) generating a plant from the plant cell; and iii) selecting a regenerated transgenic plant comprising the DNA construct. Transgenic plants produced by the methods comprise a unique combination of three transgene expression cassettes in terms of orientation and position relative to each other, each with a unique combination of expression elements for optimal expression of the transgenes. Furthermore, transgenic plants produced by the methods as described herein acquire tolerance to herbicides with three different herbicide modes of action. Selecting the regenerated plant comprising the DNA construct may be done using DNA or protein detection methods as described in the present disclosure. Alternatively or additionally, selecting may consist of treating the transgenic plant or plant cell with an effective amount of at least one herbicide selected from the group consisting of benzoic acid auxins such as dicamba, inhibitors of glutamine synthetase such as glufosinate, inhibitors of EPSPS such as glyphosate, and any combination thereof.

Methods of controlling, preventing, or reducing the development of herbicide-tolerant weeds are provided. The methods comprise: a) cultivating in a crop growing environment a sugar beet plant comprising a DNA molecule or transgenes of the present disclosure or event Bv_CSM63713 that provide tolerance to herbicides with three different herbicide modes of action at a single genomic location, and b) applying to the crop growing environment at least one herbicide selected from the group consisting of dicamba, glufosinate, glyphosate, and any combination thereof, wherein the sugar beet plant is tolerant to the at least one herbicide. In some embodiments, the three different herbicide modes of action are inhibition of glutamine synthetase, benzoic acid auxin, and inhibition of EPSPS. Sugar beet plants grown from the seeds comprising the DNA molecule or transgenes of the present disclosure, or event Bv_CSM63713 are tolerant to dicamba, glufosinate, glyphosate, or any combination thereof.

The herbicide(s) used in the methods described herein can be applied alone, sequentially with or in combination with one or more herbicide(s) during the growing season. The herbicide(s) used in the methods described herein can be applied in combination with one or more herbicide(s) temporally (for example, as a tank mixture or in sequential applications), spatially (for example, at different times during the growing season including before and after sugar beet seed planting), or both. For example, a method for controlling the development of herbicide resistance in weeds is provided that comprises planting seed comprising sugar beet event Bv_CSM63713 in an area and applying an herbicidally effective amount over the growing season of one or more of benzoic acid auxins such as dicamba, inhibitors of glutamine synthetase such as glufosinate, and inhibitors of EPSPS such as glyphosate alone or in any combination with another herbicide, for the purpose of controlling the development of herbicide resistance in weeds in the area. Such application of herbicide(s) may be pre-planting (any time prior to planting seed comprising sugar beet event Bv_CSM63713, including for burn-down purposes, that is application to emerging or existing weeds prior to seed plant), pre-emergence (any time after seed comprising sugar beet event Bv_CSM63713 is planted and before plants comprising sugar beet event Bv_CSM63713 emerge), or post-emergence (any time after plants comprising sugar beet event Bv_CSM63713 emerge). Multiple applications of one or more herbicides, or a combination of herbicides together or individually, may be used over a growing season, for example, two applications (such as a pre-planting application and a post-emergence application, or a pre-emergence application and a post-emergence application) or three or more applications (such as a pre-planting application and two post-emergence applications).

Also provided are methods of reducing loci for sugar beet breeding by inserting multiple transgenes at a single genomic location to provide three different modes of action for herbicide tolerance. Sugar beet event Bv_CSM63713 contains or comprises a transgenic insert comprising three independent transgene cassettes: a first expression cassette encoding a phosphinothricin N-acetyltransferase (PAT), a second expression cassette encoding a dicamba monooxygenase (DMO), and a third expression cassette encoding 5-enolpyruvylshikimate-3-phosphate synthase (EPSPS). These three transgene cassettes were inserted at a single genomic location as a contiguous polynucleotide or DNA molecule or single molecularly linked transgenic insert, and provide a commercial level of tolerance to at least one herbicide for each herbicide mode of action in a field, such as glufosinate, dicamba, glyphosate, and any combination thereof. The nucleotide sequences of the three expression cassettes are comprised in SEQ ID NO:9 and SEQ ID NO:10. As used herein, the term “commercial level” in reference to an herbicide refers to the recommended commercial rate (1×) for herbicide application for a specific herbicide. For example, for post emergence application, a 1× rate of dicamba is 0.5 lb/acre; a 1× rate of glufosinate is 0.54 lb/acre; and a 1× rate of glyphosate is 1 lb/acre. As used herein, “commercial level tolerance” refers to tolerance to one or more herbicides at the recommended commercial rates or higher as a result of transgene expression from one or more of the four expression cassettes in plants comprising event Bv_CSM63713.

Sugar beet event Bv_CSM63713 with the unique characteristics such as s single insertion site, stable integration and expression of the PAT, DMO and EPSPS transgenes, consistent and superior combinations of efficacy, including herbicide tolerance and agronomic performance, in and across multiple environment conditions in different geographies can be bred or introgressed into elite lines or varieties as a single locus by conventional breeding methods, and maintained over subsequent generations following Mendelian inheritance of single locus. Therefore, the methods of the present disclosure allow for rapid trait integration of the multiple transgenes on segregating material, saving time and resources in a breeding program and enabling rapid development of lines, compared to cases where the individual transgenes are inserted into two or more loci, necessitating tedious and laborious multiple crosses over multiple generations to select for plants comprising the multiple genes. The newly introgressed or integrated DNA molecule or polynucleotide of event Bv_CSM63713 comprising SEQ ID NO:9 and/or SEQ ID NO:10 will maintain the expression characteristics of the transgenes, and the genomic flanking sequences and chromosomal location, where it will confer tolerance to the at least one herbicide for each herbicide mode of action in a field, such as glufosinate, dicamba, glyphosate, and any combination thereof.

### Deposit Information

A deposit of a representative sample of sugar beet seed comprising event Bv_CSM63713 has been made on Aug. 10, 2021 according to the Budapest Treaty with the American Type Culture Collection (ATCC) Patent Repository having an address at 10801 University Boulevard, Manassas, Virginia, 20110, USA. The ATCC Patent Deposit Designation (accession number) for seeds comprising sugar beet event Bv_CSM63713 is Accession No. PTA-127098. Access to the deposits will be available during the pendency of the application to the Commissioner of Patents and Trademarks and persons determined by the Commissioner to be entitled thereto upon request. Upon issuance of the patent, all restrictions upon availability to the public will be irrevocably removed. The deposit will be maintained in the depository for a period of thirty (30) years, or five (5) years after the last request, or for the effective life of the patent, whichever is longer, and will be replaced as necessary during that period.

### EXAMPLES

The following examples are included to more fully describe the invention. Summarized are the construction and testing of four different transformation constructs, the generation of 1803 unique transformation events, and the analysis of thousands of individual plants over five years through the rigorous molecular, agronomic, and field testing required for the creation, identification and ultimate selection of the sugar beet event Bv_CSM63713.

It should be appreciated by those of skill in the art that many modifications can be made in the specific examples which are disclosed and still obtain a similar result. Certain agents which are both chemically and physiologically related may be substituted for the agents described herein while achieving the same or similar results. All such substitutions and modifications apparent to those skilled in the art are deemed to be within the scope of the invention.

### Example 1: Construct Design, Event Production, R0 Plant Testing

This example describes the design of four different expression constructs for tolerance to dicamba, glyphosate and glufosinate herbicides through a vector stack, the production of 1803 unique sugar beet events, and testing and analysis of the resulting transgenic sugar beet plants over multiple generations.

Four expression constructs were designed and constructed. These four constructs each contained three expression cassettes in the same orientation for driving the expression of dicamba monooxygenase (DMO), CP4 5-enolpyruvulshikimate-3-phosphate synthase (EPSPS) and phosphinothricin N-acetyltransferase (PAT) transgenes. However, each construct was designed to have a unique combination of 5′ and 3′ expression elements for the operably linked DMO, CP4 EPSPS and PAT transgenes to allow for testing of different promoters, chloroplast targeting sequences, and 3′UTRs in sugar beet plants for optimal performance of the transgenes. Constructs are shown in Table 1. The four expression constructs were then each cloned into plant transformation vectors and introduced into Agrobacterium strain AGL1.

Agrobacterium-mediated transformation of four sugar beet lines was conducted using the four constructs. Lines 1 and 2 were transformed using shoot meristematic tissue following a slightly modified transformation protocol (Lindsey and Gallois, 1990); whereas lines 3 and 4 were transformed using hypocotyl and cotyledon explants through a callus-based transformation protocol modified after Kishchenko, Komarnitskii, Kuckuk (2005). Phosphinothricin (DL-PPT, Duchefa, Haarlem—The Netherlands) was used as a selective agent at the concentration of 6 mg/L.

A total of 1803 unique transformation events were produced across all four lines from the four transformation constructs. Each transformation event was generated through random insertion of the T-DNA containing the three expression cassettes at a unique location into the sugar beet genome. Rooted plants from tissue culture were transferred to soil and hardened for four weeks at 20° C. day/17° C. night with a photoperiod of 16-hour light/8-hour dark. Plants were then transferred to the greenhouse at 19° C. day and 17° C. night with a photoperiod of 18-hour light/6-hour dark for vegetative cultivation.

Table 2 and Table 3 provide a summary of total number of events produced by each line, or by each construct, respectively. Since Line 1 is the closest to elite germplasm and contains all disease resistances necessary for US field cultivation, and since it is the best-established line in tissue culture, it was used in most experiments, and hence produced the highest number of transgenic events.

TABLE 1
Description of transformation constructs

| | | Construct | | | |
|---|---|---|---|---|---|
| Cassette | Element | HT1 | HT2 | HT3 | HT4 |
| CP4 EPSPS | Promoter | P-At.Tsf1 | P-CUCme.eEF1a | P-CUCme.SAMS2 | P-At.SAMS3 |
| | Enhancer | E-FMV.35S: | DaMV.FLT | — | — |
| | Leader | L-At.Tsf1 | L-CUCme.eEF1a | L-CUCme.SAMS2 | L-At.SAMS3 |
| | Intron | I-At.Tsf1 | I-CUCme.eEF1a | I-CUCme.SAMS2 | I-At.SAMS3 |
| | Transit peptide | TS-At.ShkG-CTP2 | TS-At.ShkG-CTP2 | TS-At.ShkG-CTP2 | TS-At.ShkG-CTP2 |
| | Gene of Interest | CR-AGRtu.aroA-CP4 | CR-AGRtu.aroA-CP4 | CR-AGRtu.aroA-CP4 | CR-AGRtu.aroA-CP4 |
| | 3′ UTR | T-Ps.RbcS2-E9 | T-Mt.Gapdh | T-Mt.AC145767v28 | T-Mt.AC145767v28 |
| DMO | Promoter | P-At.Ubq10 | P-At.Ubq10 | P-CUCme.Ubq1 | P-At. Ubq3 |
| | Enhancer | — | — | E-DaMV.FLT | — |
| | Leader | L-At.Ubq10 | L-At.Ubq10 | L-CUCme.Ubq1 | L-At.Ubq3 |
| | Intron | I-At.Ubq10 | I-At.Ubq10 | I-CUCme.Ubq1 | I-At.Ubq3 |
| | Transit peptide | TS-Ps.RbcS-3C + 27AA | TS-Ps.RbcS-3C + 27AA | TS-Ps.RbcS-3C + 27AA | TS-Ps.RbcS-3C + 27AA |
| | Gene of Interest | CR-STEma.DMO | CR-STEma.DMO | CR-STEma.DMO | CR-STEma.DMO |
| | 3′ UTR | T-Mt.Sali3 | T-Mt.AC139600v16 | T-Mt.AC139600v16 | T-Mt.AC139600v16 |
| PAT | Promoter | P-Mt.Lhcb2 | P-At.Ubq3 | P-At.Cab1 | P-CaMV.35S-enh |
| | Enhancer | — | — | — | — |
| | Leader | L-Mt.Lhcb2 | L-At.Ubq3 | L-At.Cab1 | L-Ph.DnaK |
| | Intron | — | I-At.Ubq3 | — | — |
| | Transit peptide | — | | — | — |
| | Gene of Interest | CR-STRvi.Pat | CR-STRvi.Pat | CR-STRvi.Pat | CR-STRvi.Pat |
| | 3′ UTR | T-Mt.Hsp20 | T-Mt.Hsp20 | T-Mt.Hsp20 | T-Mt.PSII-T_A |

TABLE 2
Summary of transgenic events produced by genotype

| Sugar Beet | Total Number |
|---|---|
| Genotype | of Events |
| 1 | 1150 |
| 2 | 492 |
| 3 | 130 |
| 4 | 31 |

TABLE 3
Summary of transgenic events produced by construct

| | # of | # in 1^{st} | # in 2^{nd} | # in 3^{rd} |
|---|---|---|---|---|
| Construct | Events | Year Trial | Year Trial | Year Trial |
| HT1 | 376 | 6 | 1 | 0 |
| HT2 | 441 | 7 | 3 | 0 |
| HT3 | 353 | 11 | 4 | 3 |
| HT4 | 633 | 20 | 9 | 3 |

DNA from all 1803 primary transformants was isolated using a modified method of Doyle and Doyle (1990), and screened for the presence or absence of the CP4, PAT and DMO genes, Agrobacterium T-DNA left border/right border overread, and for presence of the VirD2 gene using a Kompetitive Allele Specific PCR (KASP) genotyping assay (LGC, Teddinton, UK) according to the manufacturer's standard protocols. The VirD2 gene is a gene present in Agrobacterium tumefaciens AGL1, and thus detection of VirD2 indicates the presence of Agrobacterium tumefaciens AGL1. The assay is based on competitive allele-specific PCR and enables bi-allelic scoring of single nucleotide polymorphisms (SNPs) and insertions and deletions (Indels) at specific loci. The KASP assay mix contained assay-specific non-labelled oligos: an allele-specific primer (“AlleleX” or “X”) and one common primer (“Primer Common” or “C”). The primer sets used for the CP4, PAT and DMO genes and for the Agrobacterium are listed in Table 4.

TABLE 4
KASP primer sets for CP4, PAT, DMO and Agrobacterium

| | | SEQ |
|---|---|---|
| Primer Name | Locus | ID NO. |
| sxcp4xxxs1-X | CP4 EPSPS | 16 |
| sxcp4xxxs1-C | CP4 EPSPS | 17 |
| sxpatxxxs2-X | PAT | 21 |
| sxpatxxxs2-C | PAT | 22 |
| txdmoxxxxx-X | DMO | 37 |
| txdmoxxxxx-C | DMO | 38 |
| txmonlbxxx-X | Transformation vector | 39 |
| | left border overread | |
| txmonlbxxx-C | Transformation vector | 40 |
| | left border overread | |
| txmonrbxxx-X | Transformation vector | 41 |
| | right border overread | |
| txmonrbxxx-C | Transformation vector | 42 |
| | right border overread | |
| txvir2dd04-X | Agrobacterium virD2 | 43 |
| txvir2dd04-C | Agrobacterium virD2 | 44 |

Putative transformants that were negative for CP4, PAT and DMO were discarded. Furthermore, transformants that showed positive signals for left border (LB) or/and right border (RB) overread or read through, or presence of Agrobacterium tumefaciens AGL1 after tissue culture phase were also discarded. Only events that were positive for CP4, PAT and DMO, negative for LB and/or RB overread or read through, and negative for presence of Agrobacterium tumefaciens AGL1 were maintained for further growth and testing.

The remaining events were further analyzed for transgene copy number, intactness of the transgenic insert, absence of the vector backbone sequence, transgenic insertion site in the genome, and screened for normal, fertile plants. From this initial molecular analysis and phenotypic screening, 622 R0 events were tested for R0 trait efficacy (dicamba, glyphosate and glufosinate tolerance) in the greenhouse at the 4-6 leaf stage.

Glyphosate was applied at a rate of 4 lb/acre of Roundup PowerFlex herbicide (4× rate). Glufosinate was applied at a rate of 2.16 lb/acre of Basta herbicide (4× rate). Dicamba was applied at a rate of 2 lb/acre of Mais Banvel WG herbicide (4× rate) (Table 5). Glyphosate+dicamba double mix was applied at 4× rate each, and glyphosate+dicamba+glufosinate triple mix was applied at 2.4× rate each. For the double and triple mixes, the different herbicide products were blended before application. Herbicide-induced plant injury was evaluated visually. Events that did not show good resistance against all three herbicides were discarded.

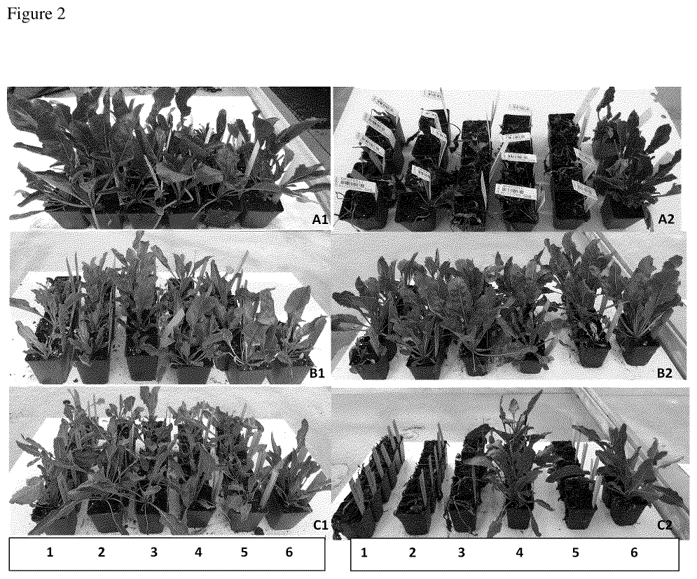

Representative results are shown in FIG. 2. Photos were taken before and two weeks after herbicide applications. Panels A1 and A2 show wild-type sugar beet before and after herbicide application; Panels B1 and B2 show event Bv_CSM63713 before and after herbicide application; and Panels C1 and C2 show another event before and after herbicide application. The herbicide treatments (from left to right) were 1. Glyphosate+glufosinate+dicamba triple mix; 2. Glyphosate+dicamba double mix; 3. Dicamba; 4. Glufosinate; 5. Glyphosate; and 6. Untreated control. As shown in FIG. 2, the wild-type sugar beet plants did not survive any of the herbicide treatments, and the plants depicted in panels C1 and C2 only survived glufosinate treatment. By contrast, the Bv_CSM63713 event showed excellent tolerance to all herbicide treatments. A single plant of the Bv_CSM63713 event showed some damage after glyphosate treatment, but this was most likely due to damage during potting before herbicide treatments.

Combining the molecular analysis data and the R0 herbicide tolerance testing, from the initial 1803 unique transformation events produced using the 4 transformation constructs and 4 transformation genotypes, 44 unique events were selected for advancement to the first-year field trials.

TABLE 5
Herbicide concentrations

| Herbicide | | 1 × Rate | 1 × Rate | 4 × Rate |
|---|---|---|---|---|
| Name | Active Ingredient | (kilogram/hectare) | (pound/acre) | (pound/acre) |
| Roundup | Glyphosate 480 g/l | 1.12 | 1 | 4 |
| PowerFlex | | | | |
| Basta | Glufosinate 183 g/l | 0.6 | 0.54 | 2.16 |
| Mais-Banvel | Dicamba 700 g/kg | 0.56 | 0.5 | 2 |
| WG | | | | |

### Example 2: First-Year Field Trials

This example describes the first-year field trials of plants containing each of the 44 selected unique events. For each unique event, thousands of plants were tested for trait efficacy (herbicide tolerance) and agronomic performance. These data were analyzed to compare the performance of each event in field conditions across all plants and all locations. The first-year field performance data in combination with molecular analysis data were then used to select superior events for advancement to second year field trials.

In the first-year field trials, 44 unique events were selected from the original 1803 events. These 44 events represented the best events from the four constructs: 6 events from HT1, 7 events from HT2, 11 events from HT3, and 20 events from HT4. Hybrid seeds were produced from the selected events via open pollination by the respective event (pollinators were either homozygous or heterozygous for the transgenes) with a sterile mother from partitions. The trait efficacy field trials used a split plot design (main plot was the chemical treatment, while the subplot was the individual events) and were conducted at two locations, each location with two replicates, in the US. Individual plots were 3.5 ft×10 ft in size, with 5 ft alleys between plots. Each plot contained 2 rows and the sowing distance was 2 inches.

In first-year trait efficacy trials, F1 hybrid plants were assessed for tolerance to dicamba, glyphosate and glufosinate. Herbicides were applied to all plants at three different stages of the sugar beet plant development: Treatment 1: 2 true leaf stage; Treatment 2: 6-8 leaf stage; and Treatment 3: 8-12 leaf stage. The efficacy trials include a total of nine treatments: 1) untreated control where weeds were manually removed by hands; 2) 2× glyphosate; 3) 4× glyphosate; 4) 2× glufosinate; 5) 4× glufosinate; 6) 2× dicamba; 7) 4× dicamba; 8) 2× dicamba and glyphosate tank mix; and 9) 4× dicamba and glyphosate tank mix (1× and 4× rates for each herbicide are provided in Table 5). At 10-14 days after each herbicide application, the percentage of herbicide-induced plant injury was evaluated visually, based on estimated growth reduction (stunted), chlorosis and necrosis.

The trait efficacy data from the first-year field trials was compiled. For each unique event, a meta-analysis of the aggregate data across both locations and all individual plants was performed for comparison of the hybrid injury ratings. Table 6 provides the average injury rating for each event for the herbicide treatment regimens across both locations. Table 7 provides a description of the injury rating scale. The results showed that on average plants from all events had low herbicide injury.

TABLE 6
Average crop injury aggregated across two locations and several ratings until 28 days after the first treatment in the first-year trials

| | | | 2x | 4x | | | | | 2x Dicamba & | 4x Dicamba & |
|---|---|---|---|---|---|---|---|---|---|---|
| | | Untreated | Glyph- | Glyph- | 2x | 4x | 2x | 4x | Glyphosate | Glyphosate |
| Construct | Event | Control | osate | osate | Glufosinate | Glufosinate | Dicamba | Dicamba | Tank Mix | Tank Mix |
| HT1 | HT1-1 | 1.3 | 1.3 | 2.9 | 3.0 | NA | 2.4 | 2.5 | 2.7 | 2.9 |
| | HT1-2 | 1.3 | 1.8 | 2.5 | 2.5 | 4.1 | 2.6 | 2.8 | 2.6 | 3.2 |
| | HT1-3 | 1.4 | 1.8 | 2.4 | 2.4 | 4.0 | 2.6 | 3.1 | 2.7 | 3.2 |
| | HT1-4 | 1.3 | 2.6 | 3.6 | 3.7 | 5.0 | 3.5 | 3.9 | 3.6 | 3.8 |
| | HT1-5 | 1.5 | 1.8 | 2.1 | 2.8 | 3.8 | 2.4 | 2.8 | 2.6 | 3.2 |
| HT3 | HT3-1 | 1.3 | 1.8 | 2.0 | 2.5 | 3.3 | 2.4 | 3.0 | 2.7 | 3.1 |
| | (Bv_CSM63713) | | | | | | | | | |
| | HT3-2 | 1.4 | 1.7 | 2.4 | 2.5 | 3.8 | 2.4 | 2.8 | 2.8 | 3.3 |
| | HT3-3 | 1.3 | 1.3 | 1.6 | 2.0 | 3.2 | 2.1 | 2.6 | 2.2 | 2.8 |
| | HT3-4 | 1.3 | 1.7 | 2.1 | 2.1 | 3.4 | 2.1 | 2.6 | 2.5 | 3.1 |
| | HT3-5 | 1.5 | 1.5 | 2.0 | 2.4 | 3.6 | 2.4 | 2.9 | 2.4 | 3.1 |
| | HT3-6 | 1.2 | 1.8 | 2.1 | 2.4 | 3.5 | 2.2 | 2.8 | 2.8 | 3.4 |
| | HT3-7 | 1.3 | 1.5 | 2.3 | 2.4 | 3.4 | 2.5 | 3.2 | 2.6 | 3.0 |
| | HT3-8 | 1.3 | 1.6 | 2.5 | 2.0 | 3.4 | 2.5 | 2.7 | 3.0 | 3.4 |
| | HT3-9 | 1.4 | 1.6 | 2.3 | 1.9 | 3.3 | 2.1 | 3.0 | 2.4 | 3.1 |
| | HT3-10 | 1.4 | 1.3 | 1.9 | 2.1 | 3.0 | 2.1 | 2.2 | 2.2 | 3.2 |
| | HT3-11 | 1.5 | 1.9 | 2.3 | 2.4 | 3.2 | 2.0 | 2.9 | 2.4 | 3.4 |
| HT2 | HT2-1 | 1.7 | 1.8 | 2.3 | 2.7 | 4.5 | 2.6 | 3.0 | 3.0 | 3.5 |
| | HT2-2 | 2.1 | 1.9 | 2.5 | 2.6 | 4.1 | 2.2 | 2.7 | 2.3 | 3.2 |
| | HT2-3 | 1.4 | 1.2 | 1.8 | 1.9 | 3.0 | 1.9 | 2.3 | 2.0 | 2.6 |
| | HT2-4 | 1.4 | 2.5 | 2.3 | 2.6 | 4.0 | 2.5 | 2.9 | 2.9 | 3.4 |
| | HT2-5 | 1.4 | 1.6 | 2.6 | 2.6 | 3.9 | 2.6 | 2.7 | 2.6 | 3.4 |
| | HT2-6 | 1.4 | 1.4 | 1.9 | 2.5 | 3.8 | 2.3 | 2.8 | 2.5 | 3.3 |
| | HT2-7 | 1.5 | 1.6 | 1.8 | 2.3 | 3.8 | 2.2 | 2.9 | 2.6 | 3.1 |
| HT4 | HT4-1 | 1.5 | 1.8 | 2.1 | 2.5 | 3.8 | 2.5 | 3.1 | 2.9 | 3.5 |
| | HT4-2 | 1.7 | 1.9 | 2.2 | 2.5 | 3.1 | 2.0 | 2.7 | 2.4 | 3.1 |
| | HT4-3 | 2.2 | 1.7 | 2.7 | 2.6 | 3.5 | 2.4 | 3.4 | 3.0 | 3.7 |
| | HT4-4 | 1.5 | 1.5 | 2.0 | 2.2 | 3.0 | 2.1 | 3.1 | 2.6 | 3.8 |
| | HT4-5 | 2.5 | 2.7 | 3.5 | 3.3 | 4.3 | 3.4 | 3.7 | 3.4 | 4.3 |
| | HT4-6 | 1.3 | 1.6 | 2.0 | 2.1 | 2.4 | 1.8 | 2.6 | 2.0 | 2.8 |
| | HT4-7 | 1.2 | 1.5 | 2.4 | 2.1 | 3.1 | 2.1 | 2.6 | 2.1 | 3.2 |
| | HT4-8 | 1.5 | 1.5 | 2.3 | 2.3 | 3.1 | 2.2 | 3.1 | 3.0 | 2.9 |
| | HT4-9 | 1.4 | 1.5 | 2.3 | 2.3 | 3.4 | 2.3 | 3.0 | 2.4 | 3.7 |
| | HT4-10 | 1.4 | 1.6 | 2.2 | 2.3 | 3.2 | 2.5 | 3.0 | 2.6 | 3.1 |
| | HT4-11 | 1.3 | 1.6 | 2.1 | 2.6 | 3.4 | 2.5 | 3.1 | 2.4 | 3.6 |
| | HT4-12 | 1.6 | 1.8 | 2.2 | 2.5 | 3.5 | 2.5 | 3.6 | 2.4 | 3.3 |
| | HT4-13 | 1.3 | 1.6 | 2.1 | 2.1 | 3.3 | 2.3 | 2.8 | 2.5 | 3.4 |
| | HT4-14 | 1.3 | 1.4 | 2.3 | 3.3 | 2.9 | 1.8 | 2.4 | 2.1 | 2.8 |
| | HT4-15 | 1.4 | 2.2 | 2.1 | 2.8 | 3.8 | 2.7 | 3.9 | 3.2 | 3.8 |
| | HT4-16 | 1.4 | 1.5 | 2.0 | 2.3 | 3.3 | 2.2 | 3.0 | 2.8 | 3.0 |
| | HT4-17 | 1.3 | 1.6 | 2.7 | 1.9 | 2.9 | 2.3 | 3.3 | 2.4 | 3.0 |
| | HT4-18 | 1.4 | 1.4 | 1.9 | 2.5 | 3.3 | 2.3 | 2.8 | 2.5 | 3.1 |
| | HT4-19 | 1.5 | 1.7 | 2.5 | 2.5 | 3.1 | 2.5 | 3.2 | 2.8 | 3.5 |
| | HT4-20 | 1.4 | 1.6 | 2.8 | 2.5 | 3.6 | 2.7 | 2.9 | 2.8 | 3.7 |
| | HT4-21 | 1.5 | 1.8 | 2.5 | 3.1 | 3.4 | 2.4 | 3.4 | 2.8 | 3.6 |

TABLE 7
Description of the injury scale

| Scale | Trait Injury (Plot Quality) |
|---|---|
| 1 | No symptoms/no stunted plants/no yellowing of leaves |
| 2 | Only very few plants showing light yellowing |
| 3 | <5% of the plants yellow/with chlorosis/stunted |
| 4 | <15% of the plants yellow/with chlorosis/stunted |
| 5 | <25% of the plants yellow/with chlorosis/with |
| | necrosis/stunted |
| 6 | <38% of the plants yellow/with chlorosis/stunted |
| 7 | <50% of the plants yellow/with chlorosis/with |
| | necrosis/stunted |
| 8 | <75% of the plants yellow/with chlorosis/stunted |
| 9 | Whole plot susceptible/dead |

### Example 3: Molecular Characterization

This example describes the extensive molecular characterization of selected events that was done concurrently with the field trials. The molecular characterization of each event was used to assess whether an event should be selected for advancement.

DNA and RNA analyses of events were conducted using a variety of techniques known in the art. Southern blot analysis and qPCR were performed on genomic DNA to confirm the presence of a single copy of the entire transgene without any vector backbone sequence. DNA amplification and sequencing were used to confirm the composition and intactness of the insert sequence in the transgenic insert for each event. The DNA flanking each end of the transgenic insert (the 5′ and 3′ ends) was sequenced, and the respective junctions were determined. Northern analysis was done to detect and measure mRNA transcripts of the CP4 gene, the DMO gene and the PAT gene in transgenic plants for each event.

Protein analysis of plants comprising each event was conducted using techniques known in the art. N-terminal protein sequencing of the CP4, DMO, and PAT proteins purified from transgenic plants containing each event was done to confirm the recombinant protein sequences. Western blot analysis was conducted on protein extracts from plants containing each event to confirm the production of the CP4, DMO, and PAT proteins.

The insertion site of each event in the genome was analyzed. The flanking sequences were used for bioinformatic analysis of the chromosomal location of the insert, and the insertion site for each event was mapped to the sugar beet genome. DNA amplification across the wild-type allele in the genome was conducted using primers specific to the flanking regions of each event. The wild-type insertion site sequence was used to map the unique site of the transgene integration for the event to the sugar beet reference genome.

Based on the comprehensive and in-depth molecular characterization for each event in combination with the trait efficacy performance data from the first-year field trials, a total of 17 unique events from the 44 tested in the first-year field trials were selected for advancement.

### Example 4: Second-Year Field Trials

This example describes the second-year field trials on plants containing each of the 17 unique events advanced from the first-year field trials for constructs HT1, HT2, HT3 and HT4. For each unique event, many individual plants were tested in the field at two locations for trait efficacy (dicamba, glyphosate and glufosinate tolerance), agronomic performance and yield. These data were analyzed to compare the performance of each event under field conditions across all plants and both locations. The data from the second-year field trials were then used to select superior events for advancement to the third-year field trials.

Hybrid F1 seeds (hemizygous for the events) for the second-year field trials were produced by open-pollinating a sterile female parent from partition with the selected events. R2 seeds from five advanced lines (homozygous for the events) were also produced via open pollination in partitions for testing in the field trials. The trait efficacy field trials used a split plot design (main plot was the chemical treatment, while the subplot was the individual events) and were conducted at two locations, each location with two replicates, in the US. Individual plots were 3.5 ft×10 ft in size, with 5 ft alleys between plots. Each plot contained 2 rows and the sowing distance was 2 inches.

In the second-year trait efficacy trials, 17 unique events were selected for testing representing one event for construct HT1, three events for construct HT2, four events for construct HT3 and nine events for construct HT4. Plants from these events were assessed for tolerance to dicamba, glyphosate and glufosinate. Herbicides were applied to all plants at three different stages of the sugar beet plant development: Treatment 1: 2 true leaf stage; Treatment 2: 6-8 leaf stage; and Treatment 3: 8-12 leaf stage. The efficacy trials include a total of ten treatments: 1) untreated control; 2) 2× dicamba; 3) 2× glufosinate; 4) 2× glyphosate; 5) 2× glyphosate and dicamba tank mix; 6) 2× glyphosate and dicamba tank mix followed by 2× glufosinate; 7) 4× dicamba; 8) 1× glufosinate; 9) 4× glufosinate; and 10) 4× glyphosate (1× and 4× rates for each herbicide are provided in Table 5). At 10-14 days after each herbicide application, the percentage of herbicide induced plant injury was evaluated visually, based on estimated growth reduction (stunted), chlorosis, and necrosis.

The trait efficacy data from the second-year field trials was compiled. For each unique event, a meta-analysis of the aggregate data across both locations and all individual plants was performed for comparison of the hybrid and homozygous line (when applicable) injury ratings, using the injury scale provided in Table 7. Table 8 provides the average injury rating for each event for the herbicide treatments regimens across both locations. Meta-analysis of the trait efficacy field trials showed that on average plants from all four constructs performed exceedingly well with low herbicide injury ratings.

TABLE 8
Average crop injury aggregated across 2 locations and several ratings
until 29 days after the first treatment in the second-year trials

| | | | | | | | 2x Gly + Dic | | | | |
|---|---|---|---|---|---|---|---|---|---|---|---|
| | | | | | | 2x Gly + | tank mix | | | | |
| | | Untreated | 2x | 2x | 2x | Dic tank | followed | 4x | 1x | 4x | 4x |
| Construct | Event | Control | Dic* | Glu* | Gly* | mix | by 2x Glu | Dic | Glu | Glu | Gly |
| HT1 | HT1-6 | 1.1 | 2.6 | 2.7 | 1.7 | 2.6 | 3.0 | 2.8 | 3.1 | NA | 2.0 |
| HT3 | HT3-1 | 1.3 | 3.1 | 3.7 | 2.5 | 3.0 | 3.7 | 3.4 | 3.0 | 4.4 | 3.3 |
| | (Bv_CSM63713) | | | | | | | | | | |
| | HT3-12 | 1.2 | 2.6 | 3.1 | 1.9 | 2.7 | 3.3 | 2.8 | 3.2 | 4.1 | 2.6 |
| | HT3-13 | 1.2 | 2.5 | 3.4 | 2.0 | 3.0 | 3.4 | 3.0 | 2.9 | 4.0 | 2.8 |
| | HT3-7 | 1.1 | 2.5 | 3.1 | 1.7 | 2.4 | 3.3 | 2.8 | 2.6 | 4.3 | 2.4 |
| HT2 | HT2-7 | 1.1 | 2.3 | 3.5 | 1.8 | 2.3 | 3.2 | 2.8 | 3.0 | 4.5 | 2.3 |
| | HT3-14 | 1.1 | 3.0 | 3.2 | 2.4 | 3.1 | 3.9 | 3.0 | 3.3 | 4.4 | 3.0 |
| | HT3-15 | 1.1 | 3.0 | 3.6 | 1.8 | 2.5 | 3.2 | 2.9 | 3.2 | 4.4 | 2.9 |
| HT4 | HT4-2 | 1.2 | 2.4 | 2.8 | 1.8 | 2.5 | 3.0 | 3.1 | 2.4 | 3.7 | 2.3 |
| | HT4-22 | 1.1 | 2.4 | 2.3 | 1.7 | 2.4 | 3.0 | 2.6 | 2.4 | 3.3 | 2.2 |
| | HT4-23 | 1.3 | 2.8 | 3.0 | 1.8 | 2.7 | 3.3 | 3.2 | 2.6 | 3.9 | 2.9 |
| | HT4-24 | 1.1 | 3.5 | 4.4 | 2.5 | 3.6 | 4.1 | 4.1 | 3.8 | 4.9 | 3.7 |
| | HT4-25 | 1.1 | 2.7 | 2.8 | 2.0 | 2.7 | 3.3 | 3.3 | 2.7 | NA | NA |
| | HT4-26 | 1.1 | 2.5 | 2.7 | 2.0 | 2.7 | 3.1 | 3.0 | 2.4 | 3.7 | 2.5 |
| | HT4-27 | 1.2 | 3.1 | 3.7 | 2.5 | 3.4 | 3.8 | 3.6 | 3.3 | 4.3 | 3.3 |
| | HT4-28 | 1.1 | 2.6 | 3.3 | 1.9 | 2.7 | 3.2 | 2.8 | 3.1 | 4.0 | 2.6 |
| | HT4-29 | 1.1 | 2.7 | 3.4 | 2.2 | 2.9 | 3.3 | 3.3 | 2.9 | 4.3 | 2.9 |

*Dic = dicamba; Gly = glyphosate; Glu = glufosinate

In the second-year field trials, agronomic performance field trials were also conducted to assess if the transgenes have any effect on yield of the events compared to their conventional counterparts. In addition, the effect of postemergence glufosinate application on yield of the events was also evaluated. Six unique events were included in the agronomic performance field trials. These include one event from construct HT2, two events from construct HT3, and three events from construct HT4. The trials used a split plot design and were conducted at three locations, each location with five replicates, in the US. Individual plots were 5.5 ft.×27 ft in size. Each plot contained 3 rows, and the sowing distance was 2 inches. The plots were thinned to a 6-inch stand.

In the agronomic performance trials of the six unique events, sugar beet root yield was measured of the harvested beets per single plot and was represented as weight in tons/hectare. Polarization/sugar content (POL) was calculated by near-infrared spectroscopy (NIRS) analysis per single plot as the percentage of the fresh matter. The sugar yield was calculated based on these two components in tons/hectare.

In the combined trial analysis of series A (conventional counter-part and null-segregant tested without glyphosate treatment), and series B (under glyphosate treatment to select for the segregating hybrids) for all three trial locations, no significant difference in sugar yield was observed among all the six unique events and their conventional counter-parts. The event Bv_CSM63713 was among the best performing events. The sugar content was significantly higher in the unique events compared to the conventional counterparts by location for all three tested locations. In the combined trial analysis there was a trend of higher sugar content for the unique events. However, the difference was not significant (Table 9). “Check” in Table 9 refers to well-known varieties (reference material) normally used to relativize the data.

In the agronomic field trials, the effect of postemergence glufosinate treatment (1×) and glyphosate treatment (1×) on yield was also evaluated on the six unique events. No phenotypic difference was observed between the two different herbicide treatments. However, a reduction in sugar yield was observed for all six events with the glufosinate treatment. The reduction in sugar yield was a result of lower root yield (Table 10). However, sugar content was not affected by the treatment.

In Tables 9 and 10, LSD stands for Least Significant Difference, which allows for determination of the amount by which two levels (for example, variety, treatment) must differ to be considered significantly different from each other at an error level of 5%. LSD measures variability/difference in the trial that may be due to specific treatment(s) such as presence/absence of a transgenic insert into the sugar beet genome (as in Table 9) or glyphosate treatment vs. glufosinate treatment (as in Table 10). The value for the LSD means that the difference for a parameter (such as Sugar Yield, or Sugar Content in Table 9) must be greater than the LSD value indicated between two different treatments to be considered significant. For example, in Table 9, Sugar Yield for the hybrid of “Tester 1×Bv_CSM63713” is 13.1, whereas that for the hybrid of “Tester 1×Line E” is 12.7. Since the difference between 13.1 and 12.7 is <1.3 (the LSD value), it is considered not significant (Table 9).

TABLE 9
Summary of yield trial data for the combined analysis across three locations without glyphosate treatment

| | | | | | | | | |
|---|---|---|---|---|---|---|---|---|
| | | | | | | 10 | 18.4 | 9.6 |
| | | | | | | Sugar Yield | Sugar Content | Root Yield |
| | | LSD | 1.3 | 2.8 | 19.5 | Relative to | Relative to | Relative to |
| | | Construct | Sugar | Sugar | Root | Conventional | Conventional | Conventional |
| Female | Male | or Check | Yield | Content | Yield | Counterpart | Counterpart | Counterpart |

Series A

| | | | | | | | | |
|---|---|---|---|---|---|---|---|---|
| Line 1 | Line A | Check | 13.4 | 14.9 | 100.2 | 106.1 | 103 | 102.9 |
| Line 2 | Line B | Check | 12.6 | 15.9 | 88.7 | 99.8 | 109.7 | 91.1 |
| Line 3 | Line C | Check | 13.1 | 14.6 | 100.3 | 103.1 | 100.9 | 103 |
| Line 4 | Line D | Check | 13.1 | 15 | 97.3 | 103.6 | 103.6 | 100 |

Series B

| | | | | | | | | |
|---|---|---|---|---|---|---|---|---|
| Tester 1 | Line E | Conventional | 12.7 | 14.5 | 97.3 | 100 | 100 | 100 |
| | | counterpart | | | | | | |
| Tester 1 | HT3-1 | HT3 | 13.1 | 15.8 | 91.8 | 103.1 | 109 | 94.3 |
| | (Bv_CSM63713) | | | | | | | |
| Tester 1 | HT2-7 | HT2 | 12.7 | 15.6 | 90 | 100.1 | 107.8 | 92.5 |
| Tester 1 | HT3-7 | HT3 | 12.9 | 15.7 | 91.1 | 101.9 | 108.4 | 93.6 |
| Tester 1 | HT4-15 | HT4 | 12.7 | 15.7 | 89.3 | 99.8 | 108.4 | 91.7 |
| Tester 1 | HT4-16 | HT4 | 13 | 15.4 | 93.9 | 102.6 | 106 | 95.6 |
| Tester 1 | HT4-2 | HT4 | 13.2 | 15.8 | 92.6 | 103.9 | 108.8 | 95.1 |

TABLE 10
Summary of yield trial data for the combined analysis across three
locations with glyphosate (Gly) or glufosinate (Glu) treatment

| | | | 0.7 | 1.4 | 0.3 | 0.2 | 2.9 | 2.3 |
|---|---|---|---|---|---|---|---|---|
| | | LSD | Sugar | Sugar | Sugar | Sugar | Root | Root |
| Female | Male | Construct | Yield_Gly | Yield_Glu | Content_Gly | Content_Glu | Yield_Gly | Yield_Glu |
| Tester 1 | HT3-1 | HT-3 | 13.05 | 12.01 | 15.8 | 15.7 | 91.8 | 85.5 |
| | (Bv_CSM63713) | | | | | | | |
| Tester 1 | HT2-7 | HT-2 | 12.66 | 11.73 | 15.6 | 15.6 | 90 | 83.8 |
| Tester 1 | HT3-7 | HT-3 | 12.89 | 12.2 | 15.7 | 15.6 | 91.1 | 87.2 |
| Tester 1 | HT4-15 | HT-4 | 12.63 | 11.67 | 15.7 | 15.6 | 89.3 | 83.4 |
| Tester 1 | HT4-16 | HT-4 | 12.98 | 12.37 | 15.4 | 15.3 | 93.9 | 89.9 |
| Tester 1 | HT4-2 | HT-4 | 13.14 | 12.42 | 15.8 | 15.7 | 92.6 | 87.9 |

### Example 5: Third-Year Field Trials

This example describes the third-year field trials on plants containing each of the six unique events advanced from the second-year field trials for constructs HT3 and HT4. For each unique event, many individual plants were tested in the field at one location for trait efficacy (dicamba, glyphosate and glufosinate tolerance), agronomic performance, and yield. These data were analyzed to compare the performance of each event under field conditions across all plants. The data from the third-year field trials in combination with the molecular analysis data were then used to select the final one event.

Hybrid F1 seeds (hemizygous for the events) for the third-year field trials were produced by open-pollinating a sterile female parent from partition with the selected events. In addition, homozygous seeds for event Bv_CSM63713 were also produced via open pollination in an isolated field. These seeds were used in the trait efficacy field trials. The trials used a split plot design (main plot was the chemical treatment, while the subplot was the individual events) and were conducted at one location with two replicates, in the US. Individual plots were 60 in×8 ft in size, with 22 in alleys between plots. Each plot contained 4 rows and the sowing distance was 2 inches.

In the third-year trait efficacy trials, six unique events were selected for testing representing three events for construct HT3 and three events for construct HT4. Plants from these events were assessed for tolerance to dicamba, glyphosate and glufosinate. Herbicides were applied to all plants at three different stages of the sugar beet plant development: Treatment 1: 2 true leaf stage; Treatment 2: 6-8 leaf stage; and Treatment 3: 8-12 leaf stage.

The efficacy trials include a total of 10 treatments: 1) 2× glyphosate; 2) 4× glyphosate; 3) 1× glufosinate; 4) 2× glufosinate; 5) 4× glufosinate; 6) untreated control; 7) 2× dicamba; 8) 4× dicamba; 9) 2× dicamba and glyphosate tank mix; and 10) 2× dicamba and glyphosate tank mix followed by 2× glufosinate (1× and 4× rates for each herbicide are provided in Table 5). At 10-14 days after each herbicide application, the percentage of herbicide induced plant injury was evaluated visually, based on estimated growth reduction (stunted), chlorosis, and necrosis.

The trait efficacy data from the third-year field trials was compiled. For each unique event, a meta-analysis of the aggregate data across the replicates and all individual plants was performed for comparison of the hybrid and homozygous line (when applicable) injury ratings. Table 11 provides the average injury rating for each event for the herbicide treatments regimens, using the injury scale provided in Table 7. Meta-analysis of the trait efficacy field trials showed that on average, plants from both constructs performed exceedingly well with low herbicide injury ratings.

TABLE 11
Average crop injury aggregated across replicates and several ratings until 40 days after the first treatment in the third-year trials

| | | | | | | | | | | | 2x Dicamba + |
|---|---|---|---|---|---|---|---|---|---|---|---|
| | | | | | | | | | | | Glyphosate |
| | | | | | | | | | | 2x | Tank Mix |
| | | | 2x | 4x | 1x | 2x | 4x | 2x | 4x | Dicamba + | Followed |
| | | Untreated | Glyph- | Glyph- | Glufos- | Glufos- | Glufos- | Di- | Di- | Glyphosate | by 2x |
| Event | Construct | Control | osate | osate | inate | inate | inate | camba | camba | Tank Mix | Glufosinate |
| HT3-1 | HT3 | 1.15 | 1.55 | 1.75 | 1.65 | 2.9 | 3.4 | 1.6 | 2.5 | 2.65 | 2.55 |
| (Bv_CSM63713) | | | | | | | | | | | |
| HT3-13 | | 1.1 | 1.1 | 1.4 | 1.1 | 2.6 | 4.3 | 1.2 | 1.4 | 2.1 | 2.6 |
| HT3-7 | | 1.2 | 1.3 | 1.5 | 1.4 | 3 | 3.8 | 2.8 | 3 | 2.3 | 3.2 |
| HT4-2 | HT4 | 1.2 | 1.3 | 1.7 | 1.6 | 2.3 | 2.8 | 1.3 | 2.4 | 2.3 | 2.8 |
| HT4-22 | | 1.1 | 1 | 1.4 | 1.7 | 2 | 2.9 | 1 | 2.6 | 2.1 | 2.3 |
| HT4-28 | | 1.1 | 1.3 | 1.9 | 1.9 | 2.7 | 3.1 | 1.1 | 1.7 | 2.3 | 2.2 |

In the third-year field trials, agronomic performance field trials were also conducted to assess if the transgenes have any effect on yield of the events compared to their conventional counterparts. Four unique events were included in the agronomic performance field trials. These include two events from construct HT3 and two events from construct HT4. The trials used a randomized complete block design and were conducted at four locations, each location with three replicates, in the US. Individual plots 5.5 ft×27 ft in size, with 8 ft alleys between plots. Each plot contained 3 rows and the sowing distance was 2 inches. The plots were thinned to a 6-inch stand.

In the agronomic performance trials of the four unique events, sugar beet root yield was measured of the harvested beets per single plot and was represented as weight in tons/hectare. Polarization/sugar content (POL) was calculated by NIRS analysis per single plot as the percentage of the fresh matter. The sugar yield was calculated based on these two components in tons/hectare. In the previous year of agronomic trials, an increase in sugar content was observed in the tested events compared to the conventional counterpart. One difference in the trials is that the herbicide tolerant events were treated with glyphosate whereas the conventional counterpart was not. In the third-year field trials, a set of four unique events was tested in the yield trials at four locations, two of which were included in the previous year yield trials. The conventional counterpart and a null-segregant from one of the events were included as controls in the trials. Glyphosate was not applied to the plants. Instead, classic sugar beet herbicides and hand weeding were applied to control the weeds. As shown in Table 12, a significant increase in sugar content was observed in all four events, consistent with the previous year's observation. The null-segregant also showed an increased sugar content. No significant difference between the transgenic events and the conventional counterpart was detected for the sugar yield and the root yield except for event HT4-22 (Table 12). LSD and “Check” in Table 12 are defined in the same manner as described above for Tables 9.

TABLE 12
Yield Trial data for the combined analysis of all four locations without glyphosate application

| | | | | | | | Sugar Yield | Sugar Content | Root Yield |
|---|---|---|---|---|---|---|---|---|---|
| | | | | | | | Relative to | Relative to | Relative to |
| | | Construct | | Sugar | Sugar | Root | Conventional | Conventional | Conventional |
| Female | Male | or Control | EP_DATA_TYPE | Yield | Content | Yield | Counterpart | Counterpart | Counterpart |
| | | | LSD | 0.7 | 0.3 | 5.3 | 7.4 | 1.7 | 8.1 |
| Line 1 | Line A | Check | | 9.8 | 14.5 | 67.8 | 102.6 | 98.8 | 103.9 |
| Line 2 | Line B | Check | | 9.3 | 15.4 | 60.3 | 97.1 | 105 | 92.4 |
| Line 3 | Line C | Check | | 9.1 | 14. | 64.6 | 95.5 | 96.2 | 99 |
| Line 4 | Line D | Check | | 9.1 | 14.6 | 62.1 | 95.2 | 99.8 | 95.2 |
| Tester 1 | Line E | Conventional | | 9.6 | 14.7 | 65.2 | 100 | 100 | 100 |
| | | counterpart | | | | | | | |
| Tester 1 | HT4-22 | Null segregant | | 9.2 | 15.2 | 60.8 | 96.6 | 103.5 | 93.2 |
| | Null_Segregant | | | | | | | | |
| Tester 1 | HT3-1 | HT3 | | 9.6 | 15 | 63.8 | 100.6 | 102.4 | 97.8 |
| | (Bv_CSM63713) | | | | | | | | |
| Tester 1 | HT4-22 | HT4 | | 8.9 | 15.2 | 58.3 | 93.1 | 103.8 | 89.3 |
| Tester 1 | HT3-13 | HT3 | | 9.5 | 15.3 | 62.2 | 99.3 | 104 | 95.4 |
| Tester 1 | HT4-2 | HT4 | | 9.3 | 15.1 | 61.2 | 96.9 | 103.1 | 93.8 |

Among the unique events in the two years of agronomic trials, event Bv_CSM63713 showed the best performance. This event was further tested to evaluate the effect of different herbicide treatments on its yield performance. The herbicide treatments included: 1) 1× dicamba pre-emergence treatment (PRE); 2) 2× dicamba applied PRE; 3) 1× glufosinate; 4) 2× glufosinate; 5) 1× glyphosate; 6) 2× glyphosate; 7) 1× dicamba/glyphosate PRE; 8) 2× dicamba/glyphosate PRE; 9) 1× dicamba/glyphosate+1× glufosinate PRE; 10) 2× dicamba/glyphosate+2× glufosinate PRE (1× rates for each herbicide are provided in Table 5). The pre-emergence treatments were only done at two of the four locations due to weather conditions. However, the tested material reacted similarly at all four locations, which allowed for a combined analysis of all four trial locations. The combined analysis showed that there was no significant difference in sugar yield, sugar content and root yield for the event between hand weeding and the different herbicide treatments, except for the 2× dicamba/glyphosate+2× glufosinate pre-emergence treatment, where a reduction in sugar yield and root yield was observed (Table 13). LSD in Table 13 is defined in the same manner described above as for Tables 9, 10, and 12.

TABLE 13
The effect of different herbicide treatments on sugar yield, sugar
content and root yield of event Bv_CSM63713 across 4 locations

| | | | | | | | Sugar Yield | Sugar Content | Root Yield |
|---|---|---|---|---|---|---|---|---|---|
| | | | | Sugar | Sugar | Root | Relative to | Relative to | Relative to |
| Female | Male | Treatment | | Yield | Content | Yield | Control | Control | Control |
| | | | LSD | 0.6 | 0.3 | 3.8 | 5.4 | 1.8 | 5.7 |
| Tester 1 | Bv_CSM63713 | Hand weeding (control) | | 10 | 15.2 | 65.6 | 101.1 | 100.7 | 100.4 |
| Tester 1 | Bv_CSM63713 | Hand weeding (control) | | 9.8 | 15 | 65.1 | 98.9 | 99.3 | 99.6 |
| Tester 1 | Bv_CSM63713 | 1x Dicamba incl. PRE | | 10.2 | 15.1 | 67.4 | 103.2 | 99.7 | 103.2 |
| Tester 1 | Bv_CSM63713 | 2x Dicamba incl. PRE | | 10 | 14.9 | 66.5 | 100.3 | 98.6 | 101.9 |
| Tester 1 | Bv_CSM63713 | 1x Glufosinate | | 9.7 | 15.1 | 63.5 | 97.4 | 100.3 | 97.1 |
| Tester 1 | Bv_CSM63713 | 2x Glufosinate | | 9.5 | 15 | 63.2 | 96.2 | 99.3 | 96.7 |
| Tester 1 | Bv_CSM63713 | 1x Glyphosate | | 10.1 | 15.1 | 66.9 | 102 | 99.6 | 102.4 |
| Tester 1 | Bv_CSM63713 | 2x Glyphosate | | 10.3 | 15.2 | 67 | 103.3 | 100.6 | 102.5 |
| Tester 1 | Bv_CSM63713 | 1x Dica/Gly incl_PRE | * | 10.2 | 15.2 | 66.7 | 102.7 | 100.6 | 102.1 |
| Tester 1 | Bv_CSM63713 | 2x Dica/Gly incl_PRE | * | 9.9 | 15 | 65.3 | 99.7 | 99.5 | 100 |
| Tester 1 | Bv_CSM63713 | 1x Dica/Gly + 1xGlu | * | 9.9 | 15.1 | 65.4 | 100.1 | 100 | 100 |
| | | incl. PRE | | | | | | | |
| Tester 1 | Bv_CSM63713 | 2x Dica/Gly + 2xGlu | * | 9 | 15 | 59.4 | 90.8 | 99.6 | 90.9 |
| | | incl PRE | | | | | | | |

* Indicates pre-treatment done at two of the four locations.

### Example 6: Final Selection of the Sugar Beet Event Bv_CSM63713

As described in the previous examples, vigorous event selection was conducted over several years to select and characterize events from 1803 unique transformation events generated from four different transformation constructs. The event selection encompassed molecular characterization, and greenhouse and field trials for efficacy, agronomic and yield performance. For molecular characterization, only events meeting the criteria of absence of Agrobacterium and left border/right border overread, presence of single copy of and intact transgenes placed on a defined genomic location, and the transgene not inserted in or close to any endogenous gene were selected to move forward for further testing. Similarly, events were selected that showed trait efficacy with no negative impact on plant growth and development, agronomic and yield performance over multiple years, in multiple locations and under a variety of growth conditions. Table 14 summarizes the results of such extensive and intensive selection process, showing the reasons for the discard of most of the events, leading to the identification and selection of the sugar beet event Bv_CSM63713 as the best event for commercial development.

TABLE 14
Summary of event selection and discard reasons

| | | # of |
|---|---|---|
| | | Discarded |
| Discard Stage | Discard Reason | Events |
| Tissue culture | Dead in tissue culture or in greenhouse | 36 |
| selection | | |
| | Persistent Agrobacterium presence | 4 |
| | Tissue culture contamination | 16 |
| | Homogeneity assay negative | 40 |
| | Sub-total | 96 |
| Molecular | Negative for transgene | 54 |
| analyses | | |
| | Left border/right border overread | 781 |
| | positive | |
| | Multiple copies of transgene | 169 |
| | T-DNA inserted in gene/close to gene | 83 |
| | Vector backbone detected | 3 |
| | Unable to determine location of | 104 |
| | insertion in the genome | |
| | Sub-total | 1194 |
| Greenhouse | Negative in herbicide tolerance test | 379 |
| selection | | |
| | Phenotype undesirable | 17 |
| | Sub-total | 396 |
| Field trial | No seed set | 116 |
| | Subtotal | 116 |
| Total discarded | | 1802 |
| events | | |

### Example 7: Molecular Characterization of the Sugar Beet Event Bv_CSM63713

This example describes the extensive molecular characterization of the sugar beet event Bv_CSM63713. The characterization included confirmation of single copy insertion at a “neutral” insertion site within the sugar beet genome, determination of the insertion site on the chromosome, identification of the transgene flanking sequences, confirmation that the insert sequence matched the transformation construct, and confirmation that there was no read-through of the transgenes on either the left border or the right border. The transgenic insert of the sugar beet event Bv_CSM63713 contains the elements and sequences described in Table 15.

DNA sequence analysis of the sugar beet event Bv_CSM63713 was performed. Southern blot analysis was conducted to confirm that plants containing sugar beet event Bv_CSM63713 contained an intact single copy of the entire transgenic insert without any transformation vector backbone sequence. The insertion site of the transgene was determined by targeted locus amplification (TLA) (Paula J P de Vree et al., 2014) based a publicly accessible sugar beet genome (The Beta vulgaris resource, http://bvseq.boku.ac.at/index.shtml). Flanking DNA on both the 5′ and the 3′ ends of the transgene insert was PCR amplified and sequenced. The sequences of the flanking DNA for sugar beet event Bv_CSM63713 were then mapped to the elite wild-type genome physical assembly obtained by whole genome sequencing. The insertion site sequence information was used for bioinformatic analysis of the chromosomal location of the event. Insertion site integrity was determined by PCR across the wild-type allele using primers specific to the flanking regions of sugar beet event Bv_CSM63713. The wild-type insertion site was used to map the unique site of transgene integration for sugar beet event Bv_CSM63713 to the sugar beet reference genome. To ensure that no alterations or mutations were introduced to any region of the transgene insert during transformation, the entire transgenic insert of sugar beet event Bv_CSM63713 was isolated from the plant and sequenced. Alignment of the recovered sequence with the sequence from the transformation vector confirmed they were identical. Sequence information for the 5′ junction, 3′ junction and the transgenic insert are provided herein as SEQ ID NOs: 1-12. The wild-type sequence at the insertion site is provided as SEQ ID NO:13. A seven nucleotide deletion was observed at the insertion site, as illustrated in the depiction of SEQ ID NO: 13 in FIG. 1.

In addition, in-depth gene prediction was carried out for the integration site and additional 50 kb of flanking region on both sites using a high quality pre-trained Augustus gene model for sugar beet. The results showed that no genes are present at the insertion site (therefore, the insertion site is referred to as a “neutral insertion site”), and no signatures of a functional gene is present in the 5′ and 3′ flanking regions.

TABLE 15
Elements and Description of Sugar Beet Event Bv_CSM63713

| | SEQ | Position in | |
|---|---|---|---|
| Element | ID NO: | SEQ ID NO: 10 | Description |
| 5′ Flanking DNA | 11 | 1-1000 | Sugar beet genomic DNA sequence flanking |
| | | | the 5′ end of the transgenic insert. |
| Left border region | 48 | 1001-1259 | Left border sequence from Agrobacterium |
| | | | tumefaciens. SEQ ID NO: 48 is the full- |
| | | | length 442-nucleotide T-DNA left border |
| | | | sequence. However, since a portion of this |
| | | | sequence was truncated at the time of the |
| | | | insertion into the sugar beet genomic DNA, |
| | | | only nucleotides 184-442 of SEQ ID NO: 48 |
| | | | are present in SEQ ID NO: 10. |
| Intervening | 49 | 1260-1311 | Sequence used in DNA cloning. |
| sequence-1 | | | |
| T-Mt.Hsp20 | 50 | 1312-1811 | 3′ untranslated region for a small heat shock |
| | | | protein (Hsp20) from Medicago truncatula. |
| Intervening | N/A | 1812-1819 | Sequence used in DNA cloning; 5′- |
| sequence-2 | | | TTAATTAA-3′. |
| CR-STRvi.Pat | 52 | 1820-2371 | Codon-optimized coding sequence for |
| | | | phosphinothricin N-acetyltransferase (PAT) |
| | | | from Streptomyces viridochromogenes. |
| Intervening | N/A | 2372-2377 | Sequence used in DNA cloning; 5′- |
| sequence-3 | | | GGTACC-3′. |
| L-At.Cab1 | 54 | 2378-2447 | The 5′ untranslated region for the |
| | | | chlorophyll A-B (Cab1) binding protein |
| | | | from Arabidopsis thaliana. |
| P-At.Cab1 | 55 | 2448-3765 | The promoter for the chlorophyll A-B (Cab1) |
| | | | binding protein gene from Arabidopsis |
| | | | thaliana. |
| Intervening | N/A | 3766-3771 | Sequence used in DNA cloning; 5′- |
| sequence-4 | | | GGATCC-3′. |
| T- | 57 | 3772-4271 | The 3′ untranslated region of a putative |
| Mt.AC139600v16 | | | protein from Medicago truncatula. |
| Intervening | N/A | 4272-4277 | Sequence used in DNA cloning; 5′- |
| sequence-5 | | | ACTAGT-3′. |
| CR-STEma.DMO | 59 | 4278-5300 | Codon-optimized coding sequence for |
| | | | dicamba monooxygenase (DMO) from |
| | | | Stenotrophomonas maltophilia. |
| TS-Ps.RbcS | 60 | 5301-5552 | The targeting sequence from a ribulose |
| | | | bisphosphate carboxylase small subunit |
| | | | (RbcS) from Pisum sativum. |
| Intervening | 61 | 5553-5563 | Sequence used in DNA cloning. |
| sequence-6 | | | |
| I-CUCme.Ubq1 | 62 | 5564-6024 | The intron for a ubiquitin protein from |
| | | | Cucumis melo. |
| L-CUCme.Ubq1 | 63 | 6025-6106 | The 5′ untranslated region for a ubiquitin |
| | | | protein from Cucumis melo. |
| P-CUCme.Ubq1 | 64 | 6107-8174 | The promoter for a ubiquitin protein from |
| | | | Cucumis melo. |
| Intervening | 65 | 8175-8184 | Sequence used in DNA cloning. |
| sequence-7 | | | |
| E-DaMV.FLT | 66 | 8185-8516 | The enhancer for inclusion body matrix |
| | | | protein from Dahlia Mosaic Virus. |
| Intervening | N/A | 8517-8522 | Sequence used in DNA cloning; 5′- |
| sequence-8 | | | CCGCGG-3′. |
| P- | 68 | 8523-9589 | The promoter of an S-adenosyl-L- |
| CUCme.SAMS2 | | | methionine synthetase (SAMS2) gene from |
| | | | Cucumis melo. |
| L- | 69 | 9590-9681 | The 5′ untranslated region of an S-adenosyl- |
| CUCme.SAMS2 | | | L-methionine synthetase (SAMS2) gene |
| | | | from Cucumis melo. |
| I- | 70 | 9682-10526 | The intron of a S-adenosyl-L-methionine |
| CUCme.SAMS2 | | | synthetase (SAMS2) gene from Cucumis |
| | | | melo. |
| Intervening | N/A | 10527-10535 | Sequence used in DNA cloning; 5′- |
| sequence-9 | | | ACGCGTACC-3′. |
| TS-At.ShkG- | 72 | 10536-10763 | The N-terminal chloroplast transit peptide |
| CTP2 | | | from 5-enolpyruvylshikimate-3-phosphate |
| | | | synthase (EPSPS) from Arabidopsis |
| | | | thaliana. |
| CR-CP4EPSPS | 73 | 10764-12131 | Codon optimized coding sequence for 5- |
| | | | enolpyruvylshikimate-3-phosphate synthase |
| | | | (EPSPS) from Agrobacterium sp. strain |
| | | | CP4. |
| Intervening | N/A | 12132-12137 | Sequence used in DNA cloning; 5′- |
| sequence-10 | | | AGGCCT-3′. |
| T- | 75 | 12138-12637 | The 3′ untranslated region of a hypothetical |
| Mt.AC145767v28 | | | protein from Medicago truncatula. |
| Intervening | 76 | 12638-12681 | Sequence used in DNA cloning. |
| sequence-11 | | | |
| Right border | 77 | 12682-12722 | Right border sequence from Agrobacterium |
| region | | | tumefaciens. SEQ ID NO: 77 is the full- |
| | | | length 357-nucleotide T-DNA left border |
| | | | sequence. However, since a portion of this |
| | | | sequence was truncated at the time of the |
| | | | insertion into the sugar beet genomic DNA, |
| | | | only nucleotides 317-357 of SEQ ID NO: 77 |
| | | | are present in SEQ ID NO: 10. |
| 3′ Flanking DNA | 12 | 12723-13722 | Sugar beet genomic DNA sequence flanking |
| | | | the 3′ end of the transgenic insert. |

RNA analysis of plants comprising the sugar beet event Bv_CSM63713 was conducted. Northern blot analysis was performed on total RNA isolated from leaf tissues of plant containing sugar beet event Bv_CSM63713. The results confirmed that the CP4, DMO, and PAT mRNA transcripts were expressed in the correct sizes and amounts as expected in the event Bv_CSM63713.

Protein analysis of plants comprising the event Bv_CSM63713 was conducted. N-terminal protein sequencing of the expressed CP4-EPSPS, DMO, and PAT proteins was performed using immunopurified protein extracts from leaf tissue to confirm the authentic N-terminal amino acid sequence. Western blot analysis was conducted on protein extracts from leaf tissue comprising the sugar beet event Bv_CSM63713 to confirm that a single expected-sized protein was being produced for CP4-EPSPS, DMO, and PAT, respectively. ELISAs were also used to determine protein levels in the leaves of plants for the CP4-EPSPS, DMO, and PAT proteins.

### Example 8: Detection of the Sugar Beet Event Bv_CSM63713

This example describes the detection of the sugar beet event Bv_CSM63713. Detection of the event in a sample can be achieved using DNA, RNA, or protein detection techniques. A qualitative detection method that is specific, robust, and easily scalable enables tracing of the specific event in research and breeding programs and helps maintain product integrity. Illustrative detection methods and materials are provided below. Detection may determine the presence or absence of the sugar beet event Bv_CSM63713 in a sample. DNA sequence information for the sugar beet event Bv_CSM63713 is provided herein as SEQ ID NOs:1-12. The transgenic insert of sugar beet event Bv_CSM63713 contains the elements described in Table 15.

### 1) Detection of the Sugar Beet Event Bv_CSM63713 by KASP

A Kompetitive Allele Specific PCR (KASP, LGC, Teddinton, UK) genotyping assay was developed to identify sugar beet event Bv_CSM63713 in a sample. The assay is based on competitive allele-specific PCR and enables bi-allelic scoring of single nucleotide polymorphisms (SNPs) and insertions and deletions (Indels) at specific loci.

The KASP Assay mix contained three assay-specific non-labelled oligos: two allele-specific forward primers and one common reverse primer. Two sets of primer combinations were developed for the event-specific KASP assays. The primers and the corresponding SEQ ID NOs are listed in Table 16. Primer set 1 txht024d01 was targeted to the 5′ region of SEQ ID NO:10, whereas primer set 2 txht024d02 was targeted to the 3′ region of SEQ ID NO:10. The event-specific assays can be performed using the two sets of primers independently, or using only one set of the primer combinations.

The KASP assay mix and the universal KASP master mix were added to DNA samples, a thermal cycling reaction was then performed, followed by an end-point fluorescent read. The allele-specific primers each harbored a unique tail sequence (underlined in Table 16) that corresponded with a universal FRET (fluorescence resonant energy transfer) cassette; one labelled with FAM dye and the other with HEX dye. The KASP Master mix contained the universal FRET cassettes, ROX passive reference dye, Taq polymerase, free nucleotides and MgCl₂ in an optimized buffer solution. In KASP PCR, during thermal cycling, the relevant allele-specific primer binds to the template and elongates, thus attaching the tail sequence to the newly synthesized strand. The complement of the allele-specific tail sequence is then generated during subsequent rounds of PCR, enabling the FRET cassette to bind to the DNA. The FRET cassette is then no longer quenched and emits fluorescence. When the sugar beet event Bv_CSM63713 was present in a sample, a fluorescent signal was produced. However, when the event was not present in a sample (such as wild type or non-transgenic plants), no fluorescent signal was produced.

Therefore, primer set txht024d01, or txht024d02, or both independently when used with the reaction methods produce a DNA amplicon that is diagnostic for sugar beet event Bv_CSM63713. The controls for this analysis should include a positive control containing the sugar beet event Bv_CSM63713, a negative control from non-transgenic sugar beet, and a negative control that contains no template DNA. Alternatively, each set of primers without the underlined tail can be used, independently or in combination, for PCR amplification and detection of the event specific amplicon using techniques known in the art.

TABLE 16
Event specific KASP primers

| Primer ID | Primer Set 1: txht024d01 |
|---|---|
| Forward primer | GAAGGTGACCAAGTTCATGCTAATAGAAAAGGATTACATGTAAGGCC |
| AlleleX | |
| (SEQ ID NO: 14; SEQ | |
| ID NO: 15: primer | |
| without the underlined | |
| tail) | |
| Reverse primer | CTGATCCATGTAGATTTCCCGGACAT |
| (SEQ ID NO: 18) | |

Primer Set 2: txht024d02

| | |
|---|---|
| Forward primer | GAAGGTGACCAAGTTCATGCTGCCTTCAGTTTAAACTATCAGTGTTTC |
| AlleleX | |
| (SEQ ID NO: 19; SEQ | |
| ID NO: 20: primer | |
| without the | |
| underlined tail) | |
| Reverse primer | AATTCTTGAACTTCGTGCACATACCTCTT |
| (SEQ ID NO: 23) | |

### 2) Detection of the Sugar Beet Event Bv_CSM63713 Using Antibodies

Another example of detection of sugar beet event Bv_CSM63713 is a detection kit comprises at least one antibody specific for at least one protein encoded by sugar beet event Bv_CSM63713. For example, such a kit may utilize a lateral flow strip comprises reagents activated when the tip of the strip is contacted with an aqueous solution. Illustrative proteins sufficient for use in antibody production are ones encoded by the sequence provided as SEQ ID NO:10, or any fragment thereof.

A protein detection method is developed to determine whether a sample is from a plant, seed, cell, or plant part (such as root) comprising sugar beet event Bv_CSM63713. At least one antibody specific for at least one protein encoded by sugar beet event Bv_CSM63713 is used to detect a protein encoded by sugar beet event Bv_CSM63713 in a sample. A detection kit comprising one or more antibodies specific for one or more proteins encoded by sugar beet event Bv_CSM63713 may utilize a lateral flow strip containing reagent activated when the tip of the strip is contacted with an aqueous solution. Samples of sugar beet tissues may be ground up and protein extracted for analysis using water or an aqueous buffer (for example, phosphate buffered saline containing detergent and bovine serum albumin). Following centrifugation, the aqueous supernatant is analyzed using the ELISA method in a sandwich format on a lateral flow strip containing an absorbent pad. Detection is activated by dipping the tip of the strip into the aqueous solution containing the sample to be tested.

The aqueous solution is carried up the strip by capillary action and solubilizes gold labeled antibodies on the strip. The gold-labeled antibodies are specific for at least one protein encoded by sugar beet event Bv_CSM63713 and will bind to an epitope on the protein in the sample to form an antibody-antigen complex. The gold labeled antibody-antigen complex is then carried up the strip to a nitrocellulose membrane. The membrane comprises a test line of immobilized antibodies that bind to a second, separate epitope on the protein encoded by sugar beet event Bv_CSM63713, causing a visible line to appear across the test strip if the protein encoded by sugar beet event Bv_CSM63713 is present in the sample.

### 3) Detection of the Sugar Beet Event Bv_CSM63713 by Real Time PCR Method

A qualitative real-time, event-specific PCR method was developed to identify the sugar beet event Bv_63713 in a sample. Qualitative detection of sugar beet genomic DNA was performed using a forward primer 2109_fwd1 (SEQ ID NO:34), a reverse primer 2109_rev1 (SEQ ID NO:35) and probe 2109_probe1 (SEQ ID NO:36). The probe was labeled with 6-carboxy-fluorescein (6-FAMm) at its 5′ end, and a Minor Grove Binder Non-Fluorescent-Quencher (MGBNFQ) at its 3′ end. The 5′ exonuclease activity of Taq DNA polymerase cleaves the probe from the 5′ end between the fluorophore and quencher. When hybridized to the target DNA strand, quencher and fluorophore are separated enough to produce a fluorescent signal, thus releasing fluorescence. Primers 2109_fwd1 and 2109_rev1 when used with the reaction methods and probe 2109_probe1 produced a DNA amplicon that is diagnostic for sugar beet event Bv_63713. Appropriate controls were used for the analysis, which included a positive control containing sugar beet event Bv_63713, a negative control from non-transgenic sugar beet and a negative control containing no template DNA. In addition, the analysis contained internal control primers and probe, specific to a single copy gene in the sugar beet genome.

An example of components of the reaction mix for sugar beet Bv_63713 event-specific PCR method is shown in Table 17. The PCR amplification reaction was performed using a Bio-Rad CFX96 Touch™ Real-Time PCR Detection System following the PCR cycling conditions listed in Table 18.

TABLE 17
Reaction Mix for Bv_63713 event-specific PCR Method

| | Stock | | Volume | Final | |
|---|---|---|---|---|---|
| Component | Concentration | | (μl) | Concentration | |
| Sigma Jumpstart Taq Ready | 2 | X | 10 | 1 | X |
| Mix | | | | | |
| ROX | 50 | X | 0.04 | 0.1 | X |
| MgCl₂ | 100 | mM | 1.1 | 5.5 | mM |
| 2109_fwd1 (SEQ ID NO: 34) | 10 | μM | 0.6 | 300 | nM |
| 2109_rev1 (SEQ ID NO: 35) | 10 | μM | 0.6 | 300 | nM |
| 2109_probe1 (SEQ ID NO: | 10 | μM | 0.3 | 150 | nM |
| 36) | | | | | |
| Template DNA | 2 | ng/μl | 5 | 0.5 | ng/μl |
| Nuclease-free water | | | 2.36 | | |
| Total | | | 20 | | |

TABLE 18
PCR cycling conditions

| | | | Temperature | Time | |
|---|---|---|---|---|---|
| Step | Stage | | (° C.) | (min) | Cycles |
| 1 | Initial denaturation | | 95 | 10:00 | 1 |
| 2 | Amplification | Denaturation | 95 | 00:15 | 40 |
| 3 | | Annealing & | 60 | 01:00 | |
| | | Extension | | | |

An example of a sugar beet endogenous gene used as an internal control is glutamine synthetase (GS). GS-specific primers and a GS-specific probe labelled with 6-FAM as reporter dye at its 5′ end, and BHQ1 as quencher at its 3′ end were used to amplify a 121 bp fragment of the glutamine synthetase gene. The primer and probe sequences are shown in Table 19. The components of the reaction mix are listed in Table 20. The PCR amplification reaction was performed using a Bio-Rad CFX96 Touch™ Real-Time PCR Detection System following the PCR cycling conditions listed in Table 18.

TABLE 19
Primer and probe sequences for the glutamine synthetase-specific PCR assay

| Name | Description | Sequence | Length |
|---|---|---|---|
| GluA3-F | Forward Primer | 5′-GACCTCCATATTACTGAAAGGAAG-3′ | 24 bp |
| (SEQ ID | | | |
| NO: 45) | | | |
| GluA3-R | Reverse Primer | 5′-GAGTAATTGCTCCATCCTGTTCA-3′ | 23 bp |
| (SEQ ID | | | |
| NO: 46) | | | |
| GluD1 | Probe | 5′-6FAM- | 27 bp |
| probe | | CTACGAAGTTTAAAGTATGTGCCGCTC-BHQ1-3′ | |
| (SEQ ID | | | |
| NO: 47) | | | |

TABLE 20
Reaction Mix for the glutamine synthetase specific PCR assay

| | Stock | | Volume | Final | |
|---|---|---|---|---|---|
| Component | Concentration | | (μl) | Concentration | |
| Sigma Jumpstart Taq Ready | 2 | X | 10.000 | 1 | X |
| Mix | | | | | |
| ROX | 50 | X | 0.040 | 0.1 | X |
| MgCl₂ | 100 | mM | 1.100 | 5.5 | mM |
| GluA3-F | 10 | μM | 0.600 | 300 | nM |
| GluA3-R | 10 | μM | 0.600 | 300 | nM |
| GluD1 probe (FAM:BHQ1) | 10 | μM | 0.300 | 150 | nM |
| Nuclease-free water | | | 2.360 | | |
| Template DNA | 2 | ng/μl | 5 | 0.5 | ng/μl |
| Total | | | 20 | | |

### Example 9: Zygosity Assays for Sugar Beet Event Bv_CSM63713

This example describes methods useful in determining the zygosity of event Bv_CSM63713. The zygosity assay determines whether a plant comprising sugar beet event Bv_CSM63713 is heterozygous or homozygous for the event or the wild-type allele. Illustrative detection methods and materials are provided below.

### 1) KASP Zygosity Assay

KASP genotyping assays for zygosity were developed based on competitive allele-specific (KASP) PCR as described in Example 8. Bi-allelic discrimination is achieved through the competitive binding of the two allele-specific forward primers. If a plant comprising the sugar beet event Bv_CSM63713 is homozygous, only one of the two possible fluorescent signals will be generated. If a plant comprising the sugar beet event Bv_CSM63713 is heterozygous, a mixed fluorescent signal will be generated.

The KASP assay mix and the universal KASP master mix were added to DNA samples, a thermal cycling reaction was then performed, followed by an end-point fluorescent read. The allele-specific primers each harbored a unique tail sequence (underlined in Table 21) that corresponded with a universal FRET (fluorescence resonant energy transfer) cassette; one labelled with FAM dye and the other with HEX dye. The KASP Master mix contained the universal FRET cassettes, ROX passive reference dye, Taq polymerase, free nucleotides and MgCl₂ in an optimized buffer solution. During thermal cycling, the relevant allele-specific primer binds to the template and elongates, thus attaching the tail sequence to the newly synthesized strand. The complement of the allele-specific tail sequence is then generated during subsequent rounds of PCR, enabling the FRET cassette to bind to the DNA. The FRET cassette is then no longer quenched and emitted fluorescence.

Two sets of primer combinations were developed for the event zygosity assays. The primers and the corresponding SEQ ID NOs are provided in Table 21. In Table 21, “AlleleX” refers to the wild-type allele, and “AlleleY” refers to the allele containing the event. Thus, the AlleleX primers detect the wild-type sequence, while the AlleleY primers detect the event. Zygosity assays can be performed using the two sets of primers independently or using only one set of the primer combinations. The assay results showed that homozygous wildtype plants produced only one signal, whereas transgenic plants homozygous for the event produced only a different signal. However, heterozygous plants for the event produced signals for both alleles.

TABLE 21
Primers for zygosity assays

| Primer ID | Primer Set 1: txht024s01 |
|---|---|
| Reverse primer AlleleX | GAAGGTGACCAAGTTCATGCTCACATACCTCTTCATGGCGAGGT |
| (SEQ ID NO: 24; SEQ ID | |
| NO: 30: primer without the | |
| underlined tail) | |
| Reverse primer AlleleY | GAAGGTCGGAGTCAACGGATTCCATGTAGATTTCCCGGACATGAA |
| (SEQ ID NO: 25; SEQ ID | |
| NO: 31: primer without the | |
| underlined tail) | |
| Forward primer | GTGGATGAAAATGCTCTAATAGAAAAGGAT |
| (SEQ ID NO: 26) | |
| | Primer Set 2: txht024s02 |
| Forward primer AlleleX | GAAGGTGACCAAGTTCATGCTTACATGTAAGGCCGACCTCGC |
| (SEQ ID NO: 27; SEQ ID | |
| NO: 32: primer without the | |
| underlined tail) | |
| Forward primer AlleleY | GAAGGTCGGAGTCAACGGATTCGCCTTCAGTTTAAACTATCAGTGTTT |
| (SEQ ID NO: 28; SEQ ID | |
| NO: 33: primer without the | |
| underlined tail) | |
| Reverse primer | TTCAACGAATTCCTACCAATTCTTGAACTT |
| (SEQ ID NO: 29) | |

### 2) Additional Zygosity Assays

Another zygosity assay is developed to determine whether a plant comprising sugar beet event Bv_CSM63713 is heterozygous or homozygous for the event or the wild-type allele. An amplification reaction assay can be designed using the sequence information provided herein. For example, such a PCR assay would include design of at least three primers: primer-1, primer-2 and primer-3, where primer-1 is specific to sugar beet genomic DNA on the 3′ flanking DNA of sugar beet event Bv_CSM63713; primer-2 is specific to sugar beet event Bv_CSM63713 transgenic insert; and primer-3 is specific to the wild-type allele. When used as a primer pair in an amplification reaction, primer-1 with primer-2 will produce a PCR amplicon specific for sugar beet event Bv_CSM63713. When used as a primer pair in an amplification reaction, primer-1 with primer-3 will produce a PCR amplicon specific for wild-type allele. In a PCR reaction performed on sugar beet genomic DNA, the respective PCR amplicons generated from primer-1+primer-2 and that generated from primer-1+primer-3 will differ in sequence and size of the amplicon. When the three primers are included in a PCR reaction with DNA extracted from a plant homozygous for sugar beet event Bv_CSM63713, only the primer-1+primer-2 amplicon (specific for the sugar beet event Bv_CSM63713) will be generated. When the three primers are included in a PCR reaction with DNA extracted from a plant heterozygous for sugar beet event Bv_CSM63713, both the primer-1+primer-2 amplicon (specific for the sugar beet event Bv_CSM63713 insert) and the primer-1+primer-3 amplicon (specific for the wild-type allele or absence of the sugar beet event Bv_CSM63713 insert) will be generated. When the three primers are mixed together in a PCR reaction with DNA extracted from a plant that is null for sugar beet event Bv_CSM63713 (that is wild-type), only the primer-1+primer-3 amplicon (specific for the wild-type allele) will be generated. The amplicons produced using the PCR reaction may be identified or distinguished using any method known in the art.

Another method to detect the presence and zygosity of sugar beet event Bv_CSM63713 in a plant sample is Southern blot analysis. One of skill in art would understand how to design a first Southern hybridization probe(s) specific for sugar beet event Bv_CSM63713 and a second Southern hybridization probe specific for a sugar beet plant which is null for the sugar beet event Bv_CSM63713 (wild-type). With Southern blot analysis, a signal detected only from the first Southern hybridization probe is indicative a plant homozygous for sugar beet event Bv_CSM63713; a signal detected from both the first and the second hybridization probes is indicative of a plant heterozygous for sugar beet event Bv_CSM63713; and a signal detected only from the second Southern hybridization probe indicates that the DNA was extracted from a plant that is null for sugar beet event Bv_CSM63713 (wild-type).

### REFERENCES

X. Chen, L. Levine and P.-Y. Kwok (1999). Fluorescence polarization in homogeneous nucleic acid analysis. Genome Res. 9:492-498.
P. Chinnadurai, D. Stojšin, K. Liu, G. E. Frierdich, K. C. Glenn, T. Geng, A. Schapaugh, K. Huang, A. E. Deffenbaugh, Z. L. Liu, L. A. Burzio (2018). “Variability of CP4 EPSPS expression in genetically engineered soybean (Glycine max L. Merrill). “Transgenic Research, 27:511-524.
J. J. Doyle and J. L. Doyle (1990). “Isolation of Plant DNA from Fresh Tissue”. Focus, 12 (1):13-15.
R. C. Edgar (2004). “MUSCLE: multiple sequence alignment with high accuracy and high throughput”. Nucleic Acids Research 32(5):1792-7.
S. Gurel, A. Pazuki, F. Aflaki, E. Gurel (2021). “Production of doubled haploid sugar beet (Beta vulgaris L.) plants trough gynogenesis. Methods Mol. Biol. 2289:313-323.
L. A. Harrison, M. R. Bailey, M. W. Naylor, J. E. Ream, B. G. Hammond, D. L. Nida, B. L. Burnette, T. E. Nickson, T. A. Mitsky, M. L. Taylor, R. L. Fuchs, S. R. Padgette (1996). “The Expressed Protein in Glyphosate-Tolerant Soybean, 5-Enolypyruvylshikimate-3-Phosphate Synthase from Agrobacterium sp. Strain CP4, Is Rapidly Digested In Vitro and Is not Toxic to Acutely Gavaged Mice”. Journal of Nutrition, 126 (3):728-740.
E. M. Kishchenko, I. K. Komarnitskii, N. V. Kuchuk (2005). “Production of transgenetic sugarbeet (Beta vulgaris L.) plants resistant to phosphinothricin”. Cell Biology International 29: 15-19.
K. Lindsey and P. Gallois (1990). “Transformation of sugarbeet (Beta vulgaris) by Agrobacterium tumefaciens”. J. of Experimental Botany 41 (5):529-536.
T. T. Nikiforov, R. B. Rendie, P. Goelet, Y.-H. Rogers, M. L. Kotewicz, S. Anderson, G. L. Trainor and M. R. Knapp (1994). Genetic bit analysis: a solid phase method for typing single nucleotide polymorphisms. Nucleic Acids Res. 22:4167-4175.
M. D. Pearson, L. Nguyen, Y. Zhao, W. L. McKenna, T. J. Morin and W. B. Dunbar (2019). Past and accurate quantification of insertion-site specific transgene levels from raw seed samples using solid-state nanopore technology. PloS One 14(12):e0226719.
J. M. Poehlman (1987) Breeding Sugar Beets. In: Breeding Field Crops. Springer, Dordrecht. pp 592-624.
N. Soltani, J. A. Dille, D. E. Robinson, C. L. Sprague, D. W. Morishita, N.C. Lawrence, A. R. Kniss, P. Jha, J. Felix, R. E. Nurse and P. H. Sikkema (2018). “Potential yield loss in sugar beet due to weed interference in the United States and Canada”. Weed Technology 32:749-753.
A. D. Taylor, L. Mataseje, C. J. Urfano, L. Schmidt, K. S. Antonation, M. R. Mulvey and C. R. Corbett (2018). Ecaluation of Oxford Nanopore's MinION Sequencing Device for Microbial Whole Genome Sequencing Applications. Scientific Reports 8:10931.
S. Tyagi and F. R. Kramer (1996). Molecular beacons: probes that fluoresce upon hybridization. Nature Biotechnology 14:303-308.
P. J. P. de Vree, E. de Wit, M. Yilmaz et al. (2014). “Targeted sequencing by proximity ligation for comprehensive variant detection and local haplotyping”. Nature Biotechnology 32 (10): 1019-1025.
Y. Wan, J. F. Petolino and J. M. Widholm. (1989). “Efficient Production of Doubled Haploid Plants Through Colchicine Treatment of Anther-Derived Maize Callus”, Theor. Appl. Genet., 77:889-892.
C. Wang, K. C. Glenn, C. Kessenich, E. Bell, L. A. Burzio, M. S. Koch, B. Li and A. Silvanovich (2016). “Safety assessment of dicamba mono-oxygenases that confer dicamba tolerance to various crops”. Regulatory Toxicology and Pharmacology, 81:171-182.
Y. Wang, Y. Zhao, A. Bollas, Y. Wang and K. F. Au (2021). Nanopore sequencing technology, bioinformatics and applications. Nature Biotechnology 39:1348-1365.
M. Winge (2000). Pyrosequencing—a new approach to DNA analysis. Innovation in Pharma. Tech. 00:18-24.
E. W. Weich, M. W. Levall (2003). “Doubled haploid production of sugar beet (Beta vulgaris L.)”. In: Maluszynski M., Kasha K. J., Forster B. P., Szarejko I. (eds) Doubled Haploid Production in Crop Plants. Springer, Dordrecht,

What is claimed is:

1. A recombinant DNA molecule comprising a sequence selected from the group consisting of SEQ ID NO:10, SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, a polynucleotide having a nucleotide sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or to the full length of SEQ ID NO: 9, and a complete complement of any of the foregoing.

2. The recombinant DNA molecule of claim 1, wherein the recombinant DNA molecule:
a. is derived from a sugar beet plant, seed, plant part, plant cell, progeny plant, or commodity product comprising sugar beet event Bv_CSM63713, a representative sample of seed comprising the event having been deposited as ATCC Accession No. PTA-127098;
b. is comprised in a sugar beet plant, seed, plant part, plant cell, or progeny plant comprising sugar beet event Bv_CSM63713, or a commodity product produced therefrom, a representative sample of seed comprising the event having been deposited as ATCC Accession No. PTA-127098;
c. is formed by the insertion of a heterologous nucleic acid molecule into the genomic DNA of a sugar beet plant or sugar beet cell; or
d. comprises an amplicon diagnostic for the presence of sugar beet event Bv_CSM63713.

3. A DNA molecule comprising a polynucleotide segment of sufficient length to function as a DNA probe that:
a. hybridizes specifically under stringent hybridization conditions with sugar beet event Bv_CSM63713 DNA in a sample, wherein detecting hybridization of the DNA molecule under the stringent hybridization conditions is diagnostic for the presence of sugar beet event Bv_CSM63713 in the sample; or
b. is specific for detecting in a sample at least one of:
i) a 5′ junction sequence between flanking sugar beet genomic DNA and the transgenic insert of sugar beet event Bv_CSM63713;
ii) a 3′ junction sequence between the transgenic insert of sugar beet event Bv_CSM63713 and flanking sugar beet genomic DNA;
iii) SEQ ID NO:9; and
iv) a fragment of SEQ ID NO:9 comprising a sufficient length of contiguous nucleotides of SEQ ID NO:9 to identify the sequence as a fragment of the transgenic insert of Bv_CSM63713.

4. The DNA molecule of claim 3, wherein:
a. the DNA probe comprises SEQ ID NO:36;
b. the DNA molecule comprises a nucleotide sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, and a complement of any of the foregoing; and/or
c. the sample is derived from a sugar beet plant, seed, plant part, plant cell, progeny plant, or commodity product.

5. A pair of DNA molecules comprising a first DNA molecule and a second DNA molecule, wherein the first and the second DNA molecules comprise a fragment of SEQ ID NO:10 or a complement thereof and function as DNA primers when used together in an amplification reaction with DNA comprising sugar beet event Bv_CSM63713 to produce an amplicon diagnostic for sugar beet event Bv_CSM63713 in a sample.

6. The pair of DNA molecules of claim 5, wherein:
a. the first and the second DNA molecules comprise SEQ ID NO:14 and SEQ ID NO:18; SEQ ID NO:15 and SEQ ID NO:18; SEQ ID NO:19 and SEQ ID NO:23; SEQ ID NO:20 and SEQ ID NO:23; SEQ ID NO:25 and SEQ ID NO:26; SEQ ID NO: 31 and SEQ ID NO:26; SEQ ID NO:33 and SEQ ID NO:29; SEQ ID NO:28 and SEQ ID NO:29; or SEQ ID NO:34 and SEQ ID NO:35; and/or
b. the amplicon comprises a nucleotide sequence selected from the group consisting of:
i. SEQ ID NO:1;
ii. SEQ ID NO:2;
iii. SEQ ID NO:3;
iv. SEQ ID NO:4;
v. SEQ ID NO:5;
vi. SEQ ID NO:6;
vii. SEQ ID NO:7;
viii. SEQ ID NO:8;
ix. SEQ ID NO:9;
x. SEQ ID NO:10; and
xi. a fragment of any of SEQ ID NO:1, SEQ ID NO:2, SEQ ID NO:3, SEQ ID NO:4, SEQ ID NO:5, SEQ ID NO:6, SEQ ID NO:7, SEQ ID NO:8, wherein
the fragment is at least 10 nucleotides in length and comprises nucleotides 1000-1001 or 12,722-12,723 of SEQ ID NO:10.

7. A method of detecting the presence of sugar beet event Bv_CSM63713 in a sample derived from a sugar beet seed, plant, plant part, plant cell, progeny plant, or commodity product, the method comprising:
a. contacting the sample with the DNA molecule that functions as a DNA probe of claim 3;
b. subjecting the sample and the DNA molecule that functions as a probe to stringent hybridization conditions; and
c. detecting the hybridization of the DNA molecule that functions as a probe to a DNA molecule in the sample,
wherein the hybridization of the DNA molecule that functions as a probe to the DNA molecule in the sample is diagnostic for the presence of sugar beet event Bv_CSM63713 in the sample.

8. A method of detecting the presence of sugar beet event Bv_CSM63713 in a sample derived from a sugar beet seed, plant, plant part, plant cell, progeny plant, or commodity product, the method comprising:
a) contacting the sample with the pair of DNA molecules of claim 5;
b) performing an amplification reaction sufficient to produce a DNA amplicon; and
c) detecting the presence of the DNA amplicon;
wherein the DNA amplicon comprises at least one of:
a 5′ junction sequence between flanking sugar beet genomic DNA and the transgenic insert of sugar beet event Bv_CSM63713,
a 3′ junction sequence between flanking sugar beet genomic DNA and the transgenic insert of sugar beet event Bv_CSM63713, SEQ ID NO: 9, and
a fragment of SEQ ID NO: 9 comprising a sufficient length of contiguous nucleotides of SEQ ID NO: 9 to identify the sequence as a fragment of the transgenic insert of Bv_CSM63713; and
wherein the presence of the DNA amplicon indicates the presence of sugar beet event Bv_CSM63713 in the sample.

9. The method of claim 8, wherein the DNA amplicon:
a. is at least 10 nucleotides in length, at least 11 nucleotides in length, at least 12 nucleotides in length, at least 13 nucleotides in length, at least 14 nucleotides in length, at least 15 nucleotides in length, at least 16 nucleotides in length, at least 17 nucleotides in length, at least 18 nucleotides in length, at least 19 nucleotides in length, at least 20 nucleotides in length, at least 25 nucleotides in length, at least 30 nucleotides in length, at least 35 nucleotides in length, at least 40 nucleotides in length, at least 45 nucleotides in length, at least 50 nucleotides in length, at least 60 nucleotides in length, at least 70 nucleotides in length, at least 80 nucleotides in length, at least 90 nucleotides in length, or at least 100 nucleotides in length; and/or
b. comprises a nucleotide sequence selected from the group consisting of SEQ ID NO:10; SEQ ID NO:9; SEQ ID NO:8; SEQ ID NO:7; SEQ ID NO:6; SEQ ID NO:5; SEQ ID NO:4; SEQ ID NO:3; SEQ ID NO:2; SEQ ID NO:1; and a fragment of any of SEQ ID NO:10, SEQ ID NO:8, SEQ ID NO:7, SEQ ID NO:6, SEQ ID NO:5, SEQ ID NO:4, SEQ ID NO:3, SEQ ID NO:2, SEQ ID NO:1 that is at least 10 nucleotides in length and comprises nucleotides 1000-1001 or 12,722-12,723 of SEQ ID NO:10.

10. A method of detecting the presence of sugar beet event Bv_CSM63713 in a sample of DNA derived from a sugar beet seed, plant, plant part, plant cell, progeny plant or commodity product, the method comprising:
a) contacting the sample with the DNA molecule of claim 3; and
b) performing a sequencing reaction to produce a target sequence,
wherein the target sequence comprises a nucleotide sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, a complete complement of any thereof, and a fragment of any of SEQ ID NO:1, SEQ ID NO:2, SEQ ID NO:3, SEQ ID NO:4, SEQ ID NO:5, SEQ ID NO:6, SEQ ID NO:7, SEQ ID NO:8, and SEQ ID NO:10 that is at least 10 nucleotides in length and comprises nucleotides 1000-1001 or 12722-12723 of SEQ ID NO:10.

11. A method of detecting the presence of sugar beet event Bv_CSM63713 in a sample derived from a sugar beet seed, plant, plant part, cell, progeny plant, or commodity product, the method comprising:
a) contacting the sample with at least one antibody specific for at least one protein encoded by sugar beet event Bv_CSM63713; and
b) detecting binding of the antibody to the protein in the sample,
wherein the binding of the antibody indicates the presence of sugar beet event Bv_CSM63713 in the sample.

12. A DNA detection kit for detecting the presence of sugar beet event Bv_CSM63713 in a sample, wherein the kit comprises the pair of DNA primers of claim 5.

13. A DNA detection kit for detecting the presence of sugar beet event Bv_CSM63713 in a sample, wherein the kit comprises the DNA molecule that functions as a probe of claim 3.

14. A protein detection kit for detecting the presence of sugar beet event Bv_CSM63713 in a sample, wherein the kit comprises at least one antibody specific for at least one protein encoded by sugar beet event Bv_CSM63713; wherein detecting binding of the at least one antibody to the at least one protein encoded by sugar beet event Bv_CSM63713 in a sample is diagnostic for the presence of sugar beet event Bv_CSM63713 in the sample.

15. A sugar beet plant, plant seed, plant part, or plant cell comprising a recombinant DNA molecule comprising a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or the full length of SEQ ID NO: 9, and a complete complement of any of the foregoing.

16. The sugar beet plant, plant seed, plant part, or plant cell of claim 15, wherein:
a. the plant, plant seed, plant part or plant cell expresses at least one herbicide tolerance gene selected from the group consisting of 5-enolpyruvylshikimate-3-phosphate synthase (EPSPS), dicamba monooxygenase (DMO), phosphinothricin N-acetyltransferase (PAT), and any combination thereof;
b. the plant, plant seed, plant part, or plant cell is tolerant to at least one herbicide selected from the group consisting of dicamba, glyphosate, glufosinate, and any combination thereof;
c. the plant, plant seed, plant part, or plant cell comprises sugar beet event Bv_CSM63713, a representative sample of seed comprising the event having been deposited under ATCC Accession No. PTA-127098;
d. the plant, plant seed, plant part, or plant cell is further defined as a progeny plant of any generation of a sugar beet plant comprising sugar beet event Bv_CSM63713, or a sugar beet plant part, seed, or plant cell derived therefrom; and/or
e. the plant part comprises a root, a beet, pollen, an anther, an ovary, an ovule, a flower, an embryo, a stem, a leaf, a microspore, a protoplast, or a callus.

17. A sugar beet plant, plant part, plant seed or plant cell that comprises sugar beet event Bv_CSM63713, a representative sample of seed comprising the sugar beet event sugar beet event Bv_CSM63713 having been deposited under ATCC Accession No. PTA-127098.

18. A method for controlling or preventing weeds in an area, the method comprising planting sugar beet comprising event Bv_CSM63713 in the area and applying an effective amount of at least one herbicide selected from the group consisting of dicamba, glyphosate, glufosinate, and any combination thereof, to control the weeds in the area without injury to the sugar beet or with less than about 10% injury to the sugar beet.

19. The method of claim 18, wherein:
a. applying the effective amount of at least one herbicide comprises applying at least two or more herbicides selected from the group consisting of dicamba, glyphosate, glufosinate, and any combination thereof over a growing season; or
b. the effective amount of dicamba is about 0.5 lb ae/acre to about 2 lb ae/acre of dicamba over a growing season; wherein the effective amount of glufosinate is about 0.4 lb ai/acre to about 2.16 lb ai/acre over a growing season; and/or wherein the effective amount of glyphosate is about 0.75 lb ae/acre to about 2.25 lb ae/acre over a growing season.

20. A method for controlling volunteer sugar beet comprising sugar beet event Bv_CSM63713 in an area, the method comprising applying an herbicidally effective amount of at least one herbicide other than dicamba, glyphosate, or glufosinate, wherein the herbicide application prevents growth of sugar beet comprising sugar beet event Bv_CSM63713.

21. The method of claim 20, wherein the herbicide other than glyphosate, dicamba or glufosinate is selected from the group consisting of paraquat, clethodim, clopyralid, desmedipham, triflusulfuron, 2,4-dichlorophenoxyacetic acid (2, 4-D), and acetolactate synthase (ALS) inhibitors such as sulfonylureas (SUs), imidazolinones, triazolopyrimidines, pyrimidinyl oxybenzoates, and sulfonylarnino carbonyl triazolinones, and any combination thereof.

22. A method of obtaining a seed or a plant of a sugar beet plant that is tolerant to dicamba, glyphosate, glufosinate or any combination thereof, the method comprising:
a) obtaining a population of progeny seeds or plants grown therefrom, at least one of which comprises sugar beet event Bv_CSM63713; and
b) identifying at least a first progeny seed or plant grown therefrom that comprises sugar beet event Bv_CSM63713.

23. The method of claim 22, wherein identifying the progeny seed or plant grown therefrom that comprises sugar beet event Bv_CSM63713 comprises:
a. the steps of:
i) growing the progeny seed to produce progeny plants;
ii) treating the progeny plants with an effective amount of at least one herbicide selected from the group consisting of dicamba, glyphosate, glufosinate, and any combination thereof; and
iii) selecting a progeny plant that is tolerant to the at least one herbicide selected from the group consisting of dicamba, glyphosate, glufosinate, and any combination thereof;
b. detecting the presence of sugar beet event Bv_CSM63713 in a sample derived from the progeny seed or plant grown therefrom; or
c. detecting the presence of at least one protein encoded by sugar beet event Bv_CSM63713 in a sample derived from the progeny seed or plant grown therefrom.

24. A method of determining the zygosity of a sugar beet plant, plant part, plant seed, or plant cell comprising sugar beet event Bv_CSM63713, the method comprising:
a) contacting a sample comprising DNA derived from the sugar beet plant, plant part, plant seed, or plant cell with a primer set capable of producing a first amplicon diagnostic for the presence of sugar beet event Bv_CSM63713 and a second amplicon diagnostic for the wild-type sugar beet genomic DNA not comprising sugar beet event Bv_CSM63713;
b) performing a nucleic acid amplification reaction; and
c) detecting the first amplicon and the second amplicon, wherein the presence of both amplicons indicates that the plant, plant part, seed or cell heterozygous for sugar beet event Bv_CSM63713, and the presence of only the first amplicon indicates that the plant, plant part, seed, or cell is homozygous for sugar beet event Bv_CSM63713.

25. The method of claim 24, wherein the primer set is selected from the group consisting of SEQ ID NO:24, SEQ ID NO:25 and SEQ ID NO:26;
SEQ ID NO:30, SEQ ID NO:31 and SEQ ID NO:26;
SEQ ID NO:27, SEQ ID NO:28 and SEQ ID NO:29; and
SEQ ID NO:32, SEQ ID NO:33 and SEQ ID NO:29.

26. A method of determining the zygosity of a sugar beet plant, plant part, plant seed, or plant cell comprising sugar beet event Bv_CSM63713, the method comprising:
a) contacting a sample comprising DNA derived from the sugar beet plant, plant part, plant seed, or plant cell with a probe set comprising at least a first probe that specifically hybridizes to sugar beet event Bv_CSM63713, and at least a second probe that specifically hybridizes to sugar beet genomic DNA that was disrupted by insertion of the heterologous DNA of sugar beet event Bv_CSM63713 but does not hybridize to sugar beet event Bv_CSM63713; and
b) hybridizing the probe set with the sample under stringent hybridization conditions, wherein detecting hybridization of only the first probe under the hybridization conditions is diagnostic for a sugar beet plant, plant part, seed or plant cell homozygous for sugar beet event Bv_CSM63713, and wherein detecting hybridization of both the first probe and the second probe under the hybridization conditions is diagnostic for a sugar beet plant, plant part, seed, or plant cell heterozygous for sugar beet event Bv_CSM63713.

27. A DNA construct comprising a first expression cassette, a second expression cassette, and a third expression cassette, wherein:
a) the first expression cassette comprises in operable linkage i) a chlorophyll A-B binding protein (Cab1) promoter and leader from Arabidopsis thaliana, ii) a phosphinothricin N-acetyltransferase (PAT) coding sequence, and iii) a small heat shock protein (Hsp20) 3′ UTR from Medicago truncatula;
b) the second expression cassette comprises in operable linkage i) an ubiquitin (Ubq1) promoter, leader and intron from Cucumis melo, ii) a ribulose bisphosphate carboxylase small subunit (RbcS) chloroplast transit peptide coding sequence from Pisum sativum, iii) a dicamba monooxygenase coding sequence (DMO), and iv) a putative protein 3′ UTR from Medicago truncatula; and
c) the third expression cassette comprises in operable linkage i) an inclusion body matrix protein enhancer from Dahlia Mosaic Virus, ii) an S-adenosyl-L-methionine synthetase (SAMS2) promoter, leader, and intron from Cucumis melo, iii) a 5-enolpyruvylshikimate-3-phosphate synthase chloroplast transit peptide (EPSPS) coding sequence from Arabidopsis thaliana, iv) a 5-enolpyruvylshikimate-3-phosphate synthase (EPSPS) coding sequence, and v) a hypothetical protein 3′ UTR from Medicago truncatula.

28. The DNA construct of claim 27, wherein the DNA construct comprises SEQ ID NO:9.

29. A method of improving tolerance to at least one herbicide selected from the group consisting of dicamba, glyphosate, glufosinate, and any combination thereof in a sugar beet plant comprising:
a) inserting the DNA construct of claim 27 into the genome of a sugar beet cell;
b) generating a sugar beet plant from the sugar beet cell; and
c) selecting a sugar beet plant comprising the DNA construct.

30. The method of claim 29, wherein the selecting comprises treating the sugar beet cell or plant with an effective amount of at least one herbicide selected from the group consisting of dicamba, glyphosate, glufosinate, and any combination thereof.

31. A sugar beet plant, plant seed, plant part, or plant cell tolerant to herbicides with three different modes of action at a single genomic location.

32. A sugar beet plant, plant seed, plant part, or plant cell comprising the DNA construct of claim 27.

33. A sugar beet plant, plant seed, plant part, or plant cell obtained by the method of claim 29.

34. A method of producing a progeny sugar beet plant comprising sugar beet event Bv_CSM63713 comprising:
a. sexually crossing a first sugar beet plant that comprises sugar beet event Bv_CSM63713 with itself or a second sugar beet plant;
b. collecting one or more seeds produced from the cross;
c. growing one or more seeds to produce one or more progeny plants; and
d. selecting at least a first progeny plant or seed comprising sugar beet event Bv_CSM63713.

35. An inbred or hybrid sugar beet plant or seed comprising sugar beet event Bv_CSM63713 produced by the method of claim 34.

36. A nonliving or nonregenerable sugar beet plant material comprising the recombinant DNA molecule of claim 1.

37. A nonliving or nonregenerable sugar beet plant material comprising:
a. the DNA construct of claim 27; or
b. sugar beet event Bv_CSM63713, a representative sample of seed comprising the sugar beet event sugar beet event Bv_CSM63713 having been deposited under ATCC Accession No. PTA-127098.

38. A commodity product comprising the recombinant DNA molecule of claim 1.

39. A commodity product comprising the DNA construct of claim 27.

40. The commodity product of claim 38, wherein:
a. the commodity product is produced from a transgenic sugar beet plant, plant part, plant seed, or plant cell comprising the sugar beet event Bv_CSM63713; and/or
b. the commodity product comprises whole or processed seeds, nonviable seeds, processed plant parts, processed plant tissues, dehydrated plant tissues, dehydrated plant parts, frozen plant tissues, frozen plant parts, plant parts processed for animal feed, fiber, pulp, pulp pellets, pulp shreds, tailings, juice, syrup, molasses, extract, raffinate, betaine, separator molasses solubles (SMS), or any other food for human consumption, viable seeds, viable plant parts (such as roots and leaves), or viable plant cells.

41. A method of producing a commodity product, the method comprising:
a) obtaining a transgenic sugar beet plant, plant part, or plant seed comprising sugar beet event Bv_CSM63713; and
b) producing a commodity product from the transgenic sugar beet plant, plant part, or plant seed.

42. A method of controlling, preventing, or reducing the development of herbicide-tolerant weeds comprising cultivating in a crop growing environment a sugar beet plant comprising transgenes that provide tolerance to herbicides with three different herbicide modes of action at a single genomic location.

43. The method of claim 42, wherein the three different herbicide modes of action are selected from the group consisting of inhibition of glutamine synthetase, inhibition of 5-enolpyruvylshikimate-3-phosphate synthase (EPSPS), and benzoic acid auxins.

44. A method for controlling, preventing, or reducing the development of herbicide-tolerant weeds comprising:
a. cultivating in a crop growing environment a sugar beet plant comprising the DNA construct of claim 27 for providing tolerance to herbicides with three different herbicide modes of action at a single genomic location; and
b. applying to the crop growing environment at least one herbicide selected from the group consisting of glufosinate, dicamba, glyphosate, and any combination thereof, wherein the sugar beet plant is tolerant to the at least one herbicide.

45. A method of reducing loci for sugar beet breeding by inserting transgenes at a single genomic location for tolerance to three different classes of herbicides.

46. The method of claim 45, wherein:
a. the transgenes are inserted as a single molecularly linked transgenic insert; or
b. the transgenes are inserted as a single molecularly linked transgenic insert and the transgenic insert provides a commercial level of tolerance to at least one herbicide for each herbicide mode of action.

47. A sugar beet plant, plant cell, plant part, or plant seed comprising a recombinant DNA construct integrated in chromosome 4, wherein the recombinant DNA construct confers tolerance to at least one herbicide selected from the group consisting of glufosinate, dicamba, glyphosate, and combinations of any thereof, and wherein the recombinant DNA construct is integrated in a position of said chromosome flanked by SEQ ID NO:11 and SEQ ID NO:12.
